(19) United States
(12) Patent Application Publication
(10) Pub. No.: US 2024/0093144 A1
(43) Pub. Date: Mar. 21, 2024

(54) DIFFERENTIAL ADHESION AND TENSION GUIDED FORMATION OF STEM CELL DERIVED EMBRYOS

(71) Applicant: California Institute of Technology, Pasadena, CA (US)

(72) Inventors: Min Bao, Pasadena, CA (US); Magdalena Zernicka-Goetz, Pasadena, CA (US)

(21) Appl. No.: 18/459,669

(22) Filed: Sep. 1, 2023

Related U.S. Application Data
(60) Provisional application No. 63/403,685, filed on Sep. 2, 2022.

(51) Int. Cl. C12N 5/073

(52) U.S. Cl. CPC C12N 5/0604; C12N 2501/603; C12N 2502/99; C12N 2506/02

(57) ABSTRACT

Disclosed herein include methods and compositions for use in generating synthetic embryos. In some embodiments, the method comprises culturing stem cells that over-express at least one Cadherin.

### RELATED APPLICATIONS

This application claims the benefit under 35 U.S.C. § 119(e) of U.S. Provisional Patent Application Ser. No. 63/403,685, filed Sep. 2, 2022, the content of this related application is incorporated herein by reference in its entirety for all purposes.

### STATEMENT REGARDING FEDERALLY SPONSORED R&D

This invention was made with government support under Grant Nos. HD100456 and HD104575 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-302456-US_Sequence Listing, created Aug. 29, 2023, which is 13,333 bytes in size. The information in the electronic format of the Sequence Listing is incorporated herein by reference in its entirety.

### BACKGROUND

### Field

The present disclosure relates generally to the field of cell culture and embryogenesis.

### Description of the Related Art

Trophectoderm Stem (TS) cells, eXtra-Embroynic eNdoderm (XEN) cells, and Embryonic Stem (ES) cells, are derived from the polar trophoectoderm (TE), primitive endoderm (PE) and epiblast (EPI) of the pre-implantation embryo, respectively. Remarkably, these stem cells can be used to build synthetic embryos (so called ETX embryos) in vitro that recapitulate different aspects of early mammalian embryo development, unlike other models. For instance, blastoids resemble the pre-implantation embryo but are restricted in their potential to develop into the post-implantation embryo. Gastruloids can mimic aspects of gastrulation, but lack the anterior-most structures of the post-implantation embryo. In contrast, ETX-embryos resemble the post-implantation embryo and recapitulate both the gene expression patterns and the cell movements typical of gastrulation. ETX-embryos develop into an egg cylinder from a random assortment of ES, TS and XEN cells. Strikingly, ES cells generate an EPI compartment, TS cells generate an ExE compartment, and XEN cells form the enveloping VE-like layer.

The self-organization of many cellular systems relies on the formation of distinct cell-cell contacts, that in turn depend on differential adhesion, via cadherin molecules, and/or differential cortical tension, due to reorganization of the actin cortical network. Indeed, synthetic genetic programs controlling the expression of distinct cadherins can direct the formation of custom multicellular structures that have illuminated fundamental principles of self-organization. However, the principles that operate in the self-assembly of in vitro embryo structures have never been examined. There is a need to understand principles whereby genetic information can be transduced into physical processes to achieve morphogenetic transitions that shape the embryo. There is a need for improving our ability to generate more complex stem cell embryo-like models or organoids.

### SUMMARY

Disclosed herein include methods of generating a synthetic embryo in vitro. In some embodiments, the method comprises: (a) providing a plurality of engineered embryonic stem cells (ESCs), wherein at least a portion of the plurality of engineered ESCs over-express E-cadherin (Cdh1); (b) providing a plurality of engineered trophoblast stem cells (TSCs), wherein at least a portion of the plurality of engineered TSCs over-express P-cadherin (Cdh3); (c) providing a plurality of extra-embryonic (XEN) cells; and (d) contacting the plurality of engineered ESCs, the plurality of engineered TSCs and the plurality of extra-embryonic (XEN) cells with a first culture media to form a co-culture; wherein the plurality of engineered ESCs and derivatives thereof, the plurality of engineered TSCs and derivatives thereof, and the plurality of XEN cells and derivatives thereof organize to form a synthetic embryo, wherein the synthetic embryo comprises one TS-derived compartment and one ES-derived compartment, and is covered by an outside XEN-derived monolayer. The method can comprise (e) replacing the first culture media with a second culture media about three days after the contacting step (d).

In some embodiments, the plurality of engineered ESCs, the plurality of engineered TSCs, and the plurality of XEN cells organize into a multicellular aggregate structure within about 12-24 hours of the contacting step (d). In some embodiments, the multicellular aggregate structure develops into a multicellular aggregate structure comprising one TS-derived compartment and one ES-derived compartment, at least partially covered by an outside XEN-derived monolayer with an efficiency of about 30% after about 12 hours following the contacting step (d). In some embodiments, the multicellular aggregate structure develops into the synthetic embryo with an efficiency of about 40% after at least 3 days following the contacting step (d).

In some embodiments, the synthetic embryo develops a single interior cavity with an efficiency of about 90%. In some embodiments, the single interior cavity develops between four and five days after the contacting step (d). In some embodiments, the multicellular aggregate structure develops into the synthetic embryo and comprises a single interior cavity, with an efficiency of about 40%. In some embodiments, the single interior cavity develops between four and five days after the contacting of step (d). In some embodiments, the multicellular aggregate structure develops into a synthetic embryo comprising a laminin-containing basement membrane with an efficiency of about 78%. In some embodiments, the laminin-containing basement membrane develops between four and five days after the contacting step (d).

In some embodiments, the synthetic embryo has a length of at least 200 μm about 72 hrs following the contacting step (d). In some embodiments, the synthetic embryo has a length of about 200 μm to about 500 μm, about 72 hours following the contacting step (d). In some embodiments, the synthetic embryo has a size of at least 4×10³ μm² about 72 hours following the contacting step (d). In some embodiments, the synthetic embryo has a size of about 6×10³ μm² to about 10×10³ μm², about 72 hours following the contacting step (d).

In some embodiments, the TS-derived compartment comprises cells that express at least one TS cell-marker. In some embodiments, the at least one TS cell-marker comprises Tfap2C, EOMES, or both. In some embodiments, the ES-derived compartment comprises cells that express at least one ES cell-marker. In some embodiments, the at least one ES cell-marker comprises Oct4. In some embodiments, the XEN-derived monolayer comprises cells that express at least one XEN cell-marker. In some embodiments, the at least one XEN cell-marker comprises Gata4, Gata6, or both. In some embodiments, the synthetic embryo resembles an egg cylinder structure, after about three days following the contacting step (d). In some embodiments, the synthetic embryo resembles a post-implantation embryo structure, after about four to five days following the contacting step (d).

In some embodiments, providing the plurality of engineered ESCs comprises: (i) providing an expression construct comprising a nucleic acid encoding E-cadherin, operably linked to at least one expression control element permitting gene expression in mammalian cells; and (ii) introducing the expression construct into ESCs in a manner permitting expression of the introduced construct in at least one of the ESCs, thereby generating at least one engineered ESC. In some embodiments, providing the plurality of engineered ESCs comprises culturing the at least one engineered ESC of (ii).

In some embodiments, providing the plurality of engineered TSCs comprises: (i) providing an expression construct comprising a nucleic acid encoding P-cadherin, operably linked to at least one expression control element permitting gene expression in mammalian cells; and (ii) introducing the expression construct into TSCs in a manner permitting expression of the introduced construct in at least one of the TSCs, thereby generating at least one engineered TSC. In some embodiments, providing the plurality of engineered TSCs comprises culturing the at least one engineered TSC of (ii).

In some embodiments, the expression control element comprises a promoter, an enhancer, a 5′ un-translated region, a 3′ un-translated region, or any combination thereof. In some embodiments, the promoter is a ubiquitous promoter. In some embodiments, the promoter is a constitutive or an inducible promoter. In some embodiments, the at least a portion of the plurality of engineered ESCs over-express E-cadherin relative to wild-type ESCs. In some embodiments, the at least a portion of the plurality of engineered TSCs over-express P-cadherin relative to wild-type TSCs. In some embodiments, the plurality of XEN cells are wild-type XEN cells. In some embodiments, none of the plurality of XEN cells are engineered to over-express E-cadherin, P-cadherin, or K-cadherin.

In some embodiments, the plurality of engineered ESCs comprises at least 5000 ESCs. In some embodiments, the plurality of engineered ESCs comprises 6000-7000 ESCs. In some embodiments, the plurality of engineered TSCs comprises at least 10000 TSCs. In some embodiments, the plurality of engineered TSCs comprises 15000-19000 TSCs. In some embodiments, the plurality of XEN cells comprises at least 5000 XEN cells. In some embodiments, the plurality of XEN cells comprises 5000-6000 XEN cells. In some embodiments, the ESCs, the TSCs, and/or the XEN cells are derived from a mammalian natural embryo. In some embodiments, the mammalian natural embryo is a mouse or human natural embryo.

In some embodiments, the co-culturing is performed in an inverted pyramidal microwell. In some embodiments, the inverted-pyramidal microwell is about 400 μm or about 800 μm in size. In some embodiments, the inverted-pyramidal microwell is about 400 μm or about 800 μm diameter. In some embodiments, the first culture media of step (d) comprises a ROCK inhibitor. The method can comprise removing the ROCK inhibitor following about 24 hr of co-culture in the first culture media.

In some embodiments, the first culture media and the second culture media each comprise a basal culture medium. In some embodiments, the basal culture medium comprises Dulbecco's Modified Eagle Medium (DMEM), DMEM Nutrient Mixture 12 (DMEM/F12), Roswell Park Memorial Institute (RPMI) medium 1640, Neurobasal®, Neurobasal® A, Connaught Medical Research Laboratory 1066 (CMRL-1066), or any combination thereof. In some embodiments, the first culture media and the second culture media each comprise a non-human serum or serum substitute thereof, a reducing agent, and an antibiotic. In some embodiments, the non-human serum or serum substitute comprises fetal bovine serum, bovine serum albumin, KnockOut™ Serum Replacement, or any combination thereof. In some embodiments, the reducing agent comprises beta-mercaptoethanol (2-ME), N-acetyl-L-cysteine, dithiothreitol (DTT), or any combination thereof. In some embodiments, the antibiotic comprises Penicillin-streptomycin, Amphotericin B, Ampicillin, Erythromycin, Gentamycin, Kanamycin, Neomycin, Nystatin, Polymyxin B, Tetracycline, Thiabendazole, Tylosin, or any combination thereof. In some embodiments, the first culture media and the second culture media each comprise N2 supplement, B27 supplement, Insulin-Transferrin-Selenium-Ethanolamine (ITS-X), GlutaMax™, non-essential amino acids, ascorbic acid, sodium pyruvate or any combination thereof. In some embodiments, the first culture media comprises DMEM, FBS, GlutaMax™, 2-ME, non-essential amino acids, sodium pyruvate, HEPES, and Penicillin-streptomycin. In some embodiments, the first culture medium comprises a ROCK inhibitor. In some embodiments, the first culture media comprises DMEM, 12.5% FBS, 2 mM GlutaMax™, 0.1 mM 2-ME, 0.1 mM non-essential amino acids, 1 mM sodium pyruvate, 0.02 M HEPES, 1% Penicillin-streptomycin, and 7.5 nM ROCK inhibitor. In some embodiments, the first culture media comprises DMEM, 12.5% FBS, 2 mM GlutaMax™, 0.1 mM 2-ME, 0.1 mM non-essential amino acids, 1 mM sodium pyruvate, 0.02 M HEPES, and 1% Penicillin-streptomycin. In some embodiments, the first culture media does not comprise ROCK inhibitor. In some embodiments, the second culture media comprises DMEM/F12, FBS, GlutaMax™, Penicillin-streptomycin, ITS-X, β-estradiol, progesterone, and N-acetyl-L-cysteine. The second culture media can comprise DMEM/F12, 20% FBS, 2 mM GlutaMax™, 1% Penicillin-streptomycin, 1×ITS-X, 8 nM β-estradiol, 200 ng/ml progesterone, and 25 mM N-acetyl-L-cysteine.

The method can comprise: (f) replacing the second culture media with a third culture media about one day after step (e). In some embodiments, the third culture media comprises DMEM/F12, KnockOut™ Serum Replacement, GlutaMax™, Penicillin-streptomycin, ITS-X, β-estradiol, progesterone, and N-acetyl-L-cysteine. In some embodiments, the third culture media comprises DMEM/F12, 30% KnockOut™ Serum Replacement, 2 mM GlutaMax™, 1% Penicillin-streptomycin, 1×ITS-X, 8 nM β-estradiol, 200 ng/ml progesterone, and 25 mM N-acetyl-L-cysteine.

Provided herein include differentiated cells obtainable by any of the methods disclosed herein. Disclosed herein include methods for determining the effect of a test agent on embryonic development. In some embodiments, the method comprises: a) providing a synthetic embryo generated by any method provided herein; b) contacting the synthetic embryo with a test agent; and c) determining the effect of the test agent on the synthetic embryo. In some embodiments, the determining comprises comparing a phenotype or a genotype of the synthetic embryo in the presence of the test agent with the phenotype or genotype of the synthetic embryo in the absence of the test agent. The method can comprise generating the synthetic embryo by any method disclosed herein. The method can comprise contacting the synthetic embryo with the test agent prior to step (d). The method can comprise determining the subsequent effect on formation of the synthetic embryo. In some embodiments, determining the effect on the formation of a multicellular aggregate structure, egg cylinder structure, and/or post-implantation structure. The method can comprise recording one or more images of the synthetic embryo. Disclosed herein include methods of investigating mechanisms involved in embryogenesis. Disclosed herein include methods of identifying a compound useful for treating a disease. In some embodiments, the method comprises contacting the synthetic embryo or a differentiated cell(s) obtainable by any of the methods provided herein with the compound. Disclosed herein include methods for diagnosing or treating a disease or disorder in a subject. In some embodiments, the method comprises use of a synthetic embryo, or a differentiated cell(s) obtainable by the methods provided herein, or any combination thereof. Also provided are methods of elucidating the role of a gene in embryo development. In some embodiments, the method comprises obtaining an ESC, a TSC, and/or a XEN cell where the gene has been modified or knocked out and culturing the cell to obtain a plurality of cells for use in the method for generating a synthetic embryo disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-FIG. 1G depict non-limiting exemplary schematics and data related to differential cadherin code in ETX and natural embryos. FIG. 1A displays an exemplary schematic showing self-organization and morphological transitions in natural and stem cell-derived (ETX) embryos. Epiblast (EPI) in the natural embryo and ES cells in the ETX embryo, trophectoderm (TE) in the natural embryo and TS cells in the ETX embryo are depicted. Also shown are primitive endoderm (PE) and visceral endoderm (VE) in the natural embryo and XEN cells in the ETX embryo. In E6.0 to E6.5, a mesodermal layer underneath the external XEN or endoderm (VE) derived layer is shown. ExE, extra-embryonic ectoderm. FIG. 1B displays non-limiting exemplary data showing a comparison of the average scRNA-seq read counts between ES and TS cells. Data points to the left (right) of the dashed lines represent transcripts enriched in TS (ES) cells by more than twofold. Points on the middle of the dashed line indicate equally expressed genes. FIG. 1C displays non-limiting exemplary data showing a comparison of the average scRNA-seq read counts between XEN and ES cells. Data points to the left (right) of the dashed lines represent transcripts enriched in ES (XEN) cells by more than twofold. Points on the middle of the dashed line indicate equally expressed genes. In FIG. 1B and FIG. 1C, cadherin- and protocadherin-related transcripts are highlighted (not including Tfap2c and Oct4). FIG. 1D displays exemplary violin plots showing Cdh1 (top), Cdh3 (middle) and Cdh6 gene expression (bottom) from scRNA-seq in natural and ETX embryos at different stages. NE45, NE55 and NE65 represent natural embryos collected at day 4.5, 5.5 and 6.5. ETX4, ETX5 and ETX6 represent ETX embryos collected at day 4, 5 and 6. FIG. 1E displays an exemplary schematic of chimera aggregation. Cadherin OE ES cells expressing H2B-rFP were aggregated with eight-cell-stage wild-type embryos. Their contribution to either EPI, PE, TE or excluded cells was assessed at E4.5 and are noted in the figure. FIG. 1F displays non-limiting exemplary images of chimeras stained for RFP, Sox17 and DNA (DAPI). Scale bars, 50 μm. The magnified images show the regions indicated by dashed boxes to the left (scale bars, 10 μm). The experiments were repeated three times. WT, wild type. FIG. 1G shows non-limiting exemplary data related to percentage of cells contributing to EPI, PE, TE or excluded cells in chimeras, as in FIG. 1E. The data are presented as violin plots. Each dot corresponds to an embryo. n=32 embryos for wild-type ES chimeras (3365 cells in total), n=16 embryos for Cdh1OE ES chimeras (1787 cells in total), n=13 embryos for Cdh3 OE ES chimeras (1574 cells in total) and n =16 embryos for Cdh6 OE ES chimeras (1894 cells in total). Statistical significance was determined by one-way ANOVA with a multiple comparison test. Numerical data are available as source data.

FIG. 2A-FIG. 2I display non-limiting exemplary data related to differential adhesion force in ETX embryos. FIG. 2A depicts an exemplary schematic showing cell-cell adhesion force measurement by AFM. FIG. 2B displays an exemplary resulting force-distance curve, following the procedure depicted in FIG. 2A, enabling quantification of the maximum adhesion force (Fₘₐₓ). FIG. 2C displays an exemplary chart showing Fₘₐₓ for the indicated homotypic and heterotypic adhesions between three different cell types. The experiments were performed three times independently. Total measured cell pairs: n=60 (ES-ES), n=177 (TS-TS), n=101 (XEN-XEN), n=124 (ES-TS), n=148 (XEN-TS) and n=134 (XEN-ES). Statistical significance was determined by one-way ANOVA with a multiple comparison test. FIG. 2D depicts exemplary schematics of weakly and strongly adherent cell pairs at force equilibrium. θ is the contact angle of the two adhering cells. FIG. 2E displays exemplary data related to the distribution of the measured contact angles at all cell-cell contacts. Total measured cell pairs: n=31 (ES-ES), n =38 (TS-TS), n=30 (XEN-XEN), n=32 (TS-ES), n=36 (XEN-TS) and n=29 (XEN-ES). N=3 for all conditions. Statistical significance was determined by one-way ANOVA with a multiple comparison test. FIG. 2F displays exemplary data related to adhesion forces between cells and different cadherins. Left, schematic showing cell-cadherin adhesion force measurement by AFM. Right, quantification of the results. n=42 (ES-E-cadherin), n=35 (ES-P-cadherin), n=41 (TS-E-cadherin) and n=37 (TS-P-cadherin). N=3 for all of the conditions. Statistical significance was determined by unpaired two-tailed Student's t-test. FIG. 2G displays an exemplary graph of Fₘₐₓ for homotypic adhesion between the three different cell types after downregulation of Cdh1 or Cdh3. n =60 (WT ES-ES), n=18 (Cdh1 KD ES-ES), n=19 (Cdh3 KD ES-ES), n=177 (wild-type TS-TS), n=20 (Cdh1 KD TS-TS), n=20 (Cdh3 KD TS-TS), n=101 (wild-type XEN-XEN), n=19 (Cdh1 KD XEN-XEN) and n=19 (Cdh3 KD XEN-XEN). N=3 for all conditions. Statistical significance was determined by one-way ANOVA with a multiple comparison test. FIG. 2H displays an exemplary heatmap of the adhesion parameter matrix, generated by sampling measured AFM adhesion forces, which parameterizes the CPM. FIG. 2I displays bootstrapping procedure to infer the distributions of conformations under the CPM (N=498). The schematic represents all of the possible sorted conformations, demonstrating that the ETX-like configuration is the most represented. Conformations observed at a frequency of <5% are grouped. MCS, Markov Chain Steps. In the box and whisker plots in FIG. 2C and FIG. 2E-FIG. 2G, the line inside the box indicates the median value and the error bars show the minimum and maximum values. Box edges indicate lower and upper quartile value.

FIG. 3A-FIG. 3H display non-limiting exemplary data showing the differential cadherin code regulates self-organization in ETX embryos. FIG. 3A shows representative images of the assembly of ETX embryos at different times. Scale bar, 50 μm. ES, Tfap2c; XEN, Gata4; TS, Oct4. FIG. 3B displays the diversity of self-assembled structures collected at day 3. Scale bar, 100 μm. Staining as in FIG. 3A. FIG. 3C shows non-limiting representative images of correctly sorted and missorted ETX structures after 3 days. The inset schematics show examples of the sorting outcomes. Scale bar, 100 μm. FIG. 3D depicts a pie chart showing the proportions of correctly sorted and missorted ETX structures at day 3. The 4000 structures analyzed contained three different stem cell types. Four independent experiments were performed. FIG. 3E displays non-limiting representative images of cell sorting resulting from combining Cdh1 or Cdh6 KD or OE XEN cells with wild-type ES and TS cells. Wild-type XEN cells provided the control. Scale bar, 100 μm. Staining as in FIG. 3A. FIG. 3F displays a graph of quantification of ETX structures with well-sorted or missorted XEN cells formed by XEN cells overexpressing (OE) Cdh1 or Cdh6 or KD for either Cdh1 or Cdh6. Total numbers of structures: n=470 (WT XEN), n=282 (Cdh1 KD XEN), n=519 (Cdh6 KD XEN), n=326 (Cdh1 OE XEN) and n=281 (Cdh6 OE XEN). N=3. The data are presented as means ±s.d. Statistical significance was determined by one-way ANOVA with a multiple comparison test. FIG. 3G displays non-limiting exemplary data related to cadherin KD in ES and TS cells. Left, representative images of ETX structures of Cdh1 and Cdh3 KD ES and TS cells. Scale bar, 100 μm. Right, quantification showing well-sorted and missorted ETX embryos under the indicated conditions. Total numbers of structures: n=4186 (control), n=2940, (Cdh1 KD ES), n=2471 (Cdh3 KD ES), n=2407 (Cdh1 KD TS) and n=2151 (Cdh3 KD TS). N=3. The data are presented as mean±s.d. Statistical significance was determined by one-way ANOVA with a multiple comparison test. FIG. 3H displays non-limiting exemplary data related to Cdh1 OE ES cells and Cdh3 OE TS cells and formation of synthetic embryos. Left, representative images of the ETX structures formed by combining Cdh1 OE ES cells with Cdh3 OE TS cells and wild-type XEN cells. ES, TS, and XEN cells are labeled as in FIG. 3A. Middle, magnified images indicating enlarged well-sorted ETX structures, as indicated by the white arrows to the left. Scale bars, 100 μm. right, quantification of the well-sorted ETX structures, n=3451 (control) and n=2348 (Cdh1 and Cdh3 OE) structures were selected from five independent experiments. The data are presented as means±s.d. Statistical significance was determined by unpaired two-tailed Student's t-test. The experiments were repeated four times in FIG. 3A-FIG. 3C and three times in FIG. 3E.

FIG. 4A-FIG. 4F display non-limiting exemplary data showing correct self-organization is necessary for proper morphogenesis. FIG. 4A depicts a time course of the assembly of ETX embryos stained to reveal E-cadherin (monochrome, see lower panels), Oct4 (ES) and Gata4 (XEN). The bottom row of images are magnifications of the images above and show E-cadherin staining around a nascent cavity, as indicated by the dashed lines around the cavity. Dashed lines also indicate the boundary between the ES and XEN compartment. Scale bar, 5 μm. FIG. 4B shows representative images showing Oct4 (ES), Gata4 (XEN), E-cadherin (monochrome, right panels) and DAPI (grey) staining in day 4 cadherin OE ETX structures formed by combining E-cadherin OE ES cells with P-cadherin OE TS cells and wild-type XEN cells. ETX structures formed by combining wild-type cells were used as a control. Scale bars, 100 μm. FIG. 4C displays a graph of a comparison and quantification of joined cavity formation in cadherin OE and control ETX structures. n=361 (control group) and n=253 (cadherin OE group). N=5 for each condition. The data are presented as mean±s.d. Statistical significance was determined by unpaired two-tailed Student's t-test. FIG. 4D shows representative images showing Oct4 (ES), Gata4 (XEN), laminin (monochrome, right panels) and DAPI nuclear staining in day 4 cadherin OE ETX structures formed by combining E-cadherin OE ES cells with P-cadherin OE TS cells and wild-type XEN cells. ETX structures formed by combining wild-type cells were used as a control. Scale bars, 100 μm. FIG. 4E shows a graph of quantification of the structures that contained continuous or discontinuous laminin. n=40 ETX structures per condition. N=3. The data are presented as mean ±s.d. Statistical significance was determined by unpaired two-tailed Student's t-test. FIG. 4F displays a schematic of the self-organization principles in stem cell-derived ETX embryos as disclosed herein. Differential expression of E-, K- and P-cadherins enables the sorting of ES (epiblast-like), XEN (VE-like) and TS (TE-like) stem cells. Wild-type ES cells with low E-cadherin expression and wild-type TS cells with low P-cadherin expression exhibited detrimental global sorting efficiency. This can be overcome by overexpressing E-cadherin in ES cells and P-cadherin in TS cells to increase the efficiency of ETX embryo formation. Proper morphogenesis, including cavity formation, basement membrane formation (shown as a layer between the external PE/XEN derived layer and the interal EPI/ES and TE/TS derived compartments) and symmetry breaking can only be observed in well-sorted structures.

FIG. 5A-FIG. 5F display non-limiting exemplary data related to the differential cadherin code in ETX-embryo and natural embryo. FIG. 5A displays a UMAP dimensional reduction showing Cdh1, Cdh3 and Cdh6 expression profile in different clusters as indicated by dashed lines. Each dot represents a single cell that is shaded by sample type. FIG. 5B displays a heatmap showing average expression of cadherin and protocadherin related genes revealed by scRNA-seq in natural embryos (NE, n=50) collected at 4.5, 5.5 and 6.5 days after fertilization and well-sorted ETX embryos (n=50) at 4, 5, 6 days of culture. FIG. 5C displays data related to cadherin expression. Images show colonies of cultured ES and TS cells stained to reveal E-cadherin and P-cadherin. Graphs display quantifications showing the mean intensity (A.U.) of E-cadherin or P-cadherin at cell-cell junctions. 20 colonies from 3 different experiments were selected for quantification. Scale bars represent 100 μm. Data are presented as mean±SD. Statistics calculated by unpaired two-tailed Student's-t test. FIG. 5D displays data related to cadherin expression in ETX and Natural embryos. Images display natural embryos (E5.5) and ETX embryos (Day 4) stained to reveal E-cadherin and P-cadherin. Magnified insets show E- or P-cadherin staining in ExE and EPI compartments in natural embryos, TS and ES compartments in ETX embryos. Graphs display quantifications showing the mean intensity (A.U.) of E-cadherin or P-cadherin at cell-cell junctions. n=20 ETX embryos and n=19 natural embryos were used for quantification. Data are presented as mean±SD. Statistics calculated by unpaired two-tailed Student's t test. Scale bars represent 100 pm (main image) and 20 μm (inset). FIG. 5E-FIG. 5F display representative images of E4.5 chimeras (8-cell stage embryos aggregated with Cdh6 (FIG. 5E) or Cdh3 OE ES (FIG. 5F) stained for RFP, Sox17, and DAPI. Experiments were repeated 3 times. Scale bars represent 50 μm. Zoomed images are of regions indicated by dashed lines (scale bars represent 10 μm).

FIG. 6A-FIG. 6E display non-limiting exemplary data related to differential adhesion force in ETX embryos. FIG. 6A shows representative images of cell doublets for homotypic and heterotypic cell pairs (Gata4, Tfap2c, Oct4, F-actin). Experiments were repeated 3 times. Scale bar is 10 μm. FIG. 6B displays use of ImSAnE ‘Unrolling’ algorithm to project 3D E-cadherin staining stacks onto a 2D plane. Cell contact angles were quantified using built-in correction methods. Geometric observables as well as generally distortions in projections can be correctly quantified. Scale bar is 100 μm. FIG. 6C shows a graph of cell-cell contact angle measurements based on ImSAnE method in day 4 ETX and E5.5 natural embryos. Total measured cell pairs in ETX embryos: ES-ES: n=24; TS-TS: n=15; XEN-XEN: n=16; ES-TS: n=24; XEN-TS: n=19; XEN-ES: n=16; XENi-XENi: n=16. Total measured cell pairs in natural embryos: EPI-EPI: n=20; TE-TE: n=17; VE-VE: n=22; EPI-TE: n=24; EPI-VE: n=16; TE-VE: n=19; VEi-VEi: n=24. Data are presented as box-whisker plots, black line inside the box indicates the median value and the error bar shows min to max value. Statistics calculated by one-way ANOVA with a multiple comparison test. FIG. 6D displays an enlargement of the boundary area in a day 4 ETX embryo stained with E-cadherin, with homotypic contacts highlighted in blue (TS-TS, for example bottom right panel), red (ES-ES, for example top of the image in the top-left panel) and purple (XEN-XEN), and the heterophilic boundary interface in yellow (see, for example, the top two panels). Angles formed at tricellular junctions between different types are indicated: EX, TX and ET, angles between heterotypic contacts (ES-XEN, TS-XEN and ES-TS); EE, TT and XX, angles between homotypic contacts (ES-ES, TS-TS and XEN-XEN). XXi indicates contact angles of XEN cells at cell-medium interface. Experiments were repeated 6 times. Scale bar represents 20 μm. FIG. 6E shows graphs of E-cadherin and P-cadherin mRNA expression in cells after downregulation of E- or P-cadherin by RNAi, scrambled siRNA was used as a control. P-cadherin mRNA expression in ES cells after overexpression of P-cadherin. N=4 for all conditions. Data are presented as mean±SD. Statistics calculated by unpaired two-tailed Student's t-test.

FIG. 7A-FIG. 7I show non-limiting exemplary data related to differential cadherin code and cortical tension regulate self-organization in ETX embryos. FIG. 7A displays a time course of formation of correctly-sorted ETX embryos following seeding. 0.5-h: 0/515 structures; 12-h: 79/1292 structures; 24-h: 160/1074 structures; 48-h: 134/888; 72-h: 93/702 structures. N=3 for each condition. Data are presented as mean±SD. Statistics calculated by unpaired two-tailed Student's t-test. FIG. 7B shows live cell imaging and tracking. H2B-RFP-XEN, H2B-CFP-ES and EGFP-TS were overlaid with Imaris cell-tracking spheres. FIG. 7C shows a graph of quantification of mobility for different types of cells during self-organization. Data are presented as mean±SD at different time points. FIG. 7D displays a bar graph showing the average mobility for different cell types during self-organization at different time ranges after cell seeding. Data are presented as mean±SEM. 12 structures from 3 independent experiments were imaged for quantification. Statistics calculated by unpaired two-tailed Student's t test. FIG. 7E shows exemplary images of structures made from low (control) and high number of XEN cells, stained at day-1 and day-3. Experiments were repeated 3 times. Scale bar, 10 μm. FIG. 7F depicts a schematic of morphological transitions when using low and high number of XEN cells. FIG. 7G shows a graph of cortical stiffness measurements for indicated cell types before and after treatment with Blebbistatin (Bleb). Total measured cell numbers for each condition: ES: n=58; ES+Bleb: n=34; TS: n=68; TS+Bleb: n=31; XEN: n=68; XEN+Bleb: n=35. Data are presented as mean±SD. Statistics calculated by ANOVA with a multiple comparison test. FIG. 7H displays data from experiments in which Day 3 well-sorted ETX embryos were cultured with either blebbistatin, cytochalasin D or DMSO (control) during consolidation stage for 24 hrs and immuno-stained to reveal the indicated markers. Quantification shows the percentage of disorganized ETX structures. n =84 (Bleb treated), n=83 (Cyto D treated) and n=75 (control), N=3 for each condition. Data are presented as Mean±SD. Statistics calculated by unpaired two-tailed Student's t-test. Scale bar, 100 μm. FIG. 7I depicts CPM modelling showing the effect of XEN cell stiffness (4) on externalization efficiency (N=474). The sorting efficiency calculated for each time-point is plotted as a heat-map, overlayed with contours (dotted lines).

FIG. 8A-FIG. 8D display non-limiting exemplary data related to cadherin heterogeneity within the same cell population in ETX embryos. FIG. 8A displays pie charts that show different mis-sorted ETX embryos under the indicated conditions. n=4186 (control), n=2940 (Cdh1-KD ES), n=2471 (Cdh3-KD ES), n=2407 (Cdh1-KD TS), n=2151 (Cdh3-KD TS) structures were collected from 3 independent experiments for quantification. FIG. 8B shows exemplary images of immuno-staining of ES (upper) and TS (lower) cells to reveal E-cadherin and P-cadherin, respectively. Nuclei are stained by DAPI. Scale bars represent 100 μm. Zoomed images are of regions indicated by dashed lines. Experiments were repeated 5 times. FIG. 8C depicts flow cytometric analysis of E-cadherin in wild-type ES cells, E-cadherin knockdown ES cells and ES cells over-expressing E-cadherin (upper). Flow-cytometric analysis of P-cadherin in wild-type TS cells, P-cadherin knockdown TS cells and TS cells over-expressing P-cadherin (lower). CV (coefficient of variation) values shown against peak values in plots. FIG. 8D displays indicated FACS profiles. Top: FACS profiles for E-cadherin in E-cadherin KD, WT and E-cadherin OE ES cells, Bottom: FACS profiles for P-cadherin in P-cadherin KD, WT and P-cadherin OE TS cells.

FIG. 9A-FIG. 9D display non-limiting exemplary data showing cadherin heterogeneity affects cell positioning in ETX embryos. FIG. 9A-FIG. 9B show schematics and representative images for assembled day 4 ETX-embryos from TS cells (FIG. 9A) or ES cells (FIG. 9B) overexpressing (OE) or knocked-down (KD) for the indicated cadherins. Experiments were repeated 6 times. Scale bar represents 100 μm. P-cadherin-overexpressing TS cells and E-cadherin-overexpressing ES cells were pre-stained with Hoechst to distinguish them from cadherin knockdown cells (see bottom right panel in FIG. 9A and FIG. 9B). Scale bar represents 40 μm in zoomed panels. FIG. 9C displays exemplary images of structures made from E-cadherin- OE-ES, P-cadherin -OE-TS and XEN cells, stained at different time points to reveal ES cells (Oct4), TS cells (Tfap2c) and XEN cells (Gata4). Scale bar represents 100 μm. FIG. 9D depicts a graph of quantification of the time course of formation of correctly-sorted ETX embryos following seeding. Control: 12 h: 24/332 structures; 24 h: 83/531 structures; 48 h: 71/448 structures; 72 h: 51/378 structures. Cadherin OE: 12 h: 80/276 structures; 24 h: 139/385 structures; 48 h: 136/374 structures; 72-h: 151/455 structures. N=3 for all conditions. Data are presented as Mean±SD. Statistics calculated by unpaired two-tailed Student's t-test. P values indicate significance between control and Cadherin OE ETX at the same time point.

FIG. 10A-FIG. 10F display non-limiting exemplary data showing correct cell sorting and self-organization is necessary for proper morphogenesis. FIG. 10A-FIG. 10B show comparisons and quantifications of cavity formation in structures containing mis-sorted ES or TS (FIG. 10A) and mis-sorted XEN cells (FIG. 10B). Well-sorted structures: n=73; Mis-sorted ES structures: n=103; Mis-sorted TS structures: n=109. Mis-sorted XEN structures: n=57. N=3 for each condition. Scale bar, 100 μm. Statistics calculated by unpaired two-tailed Student's t-test. FIG. 10C-FIG. 10D show the average length (FIG. 10C) and internal cavity size (FIG. 10D) of Cadherin OE ETX and control ETX at different time points. 20 to 30 structures were collected at each time point. Data are presented as mean±SD. Statistics calculated by unpaired two-tailed Student's t-test. P values indicate significant difference between Cadherin OE and control ETX at the same time point. FIG. 10E shows comparison and quantification of basement membrane formation in structures containing well-sorted and mis-sorted XEN. Well-sorted structures: n=84; Mis-sorted XEN structures: n=74. N=3 for each condition. Data are presented as mean±SD. Statistics calculated by unpaired two-tailed Student's t-test. Scale bar, 100 μm. FIG. 10F depicts a schematic image showing natural and ETX embryos use different routes to form the post-implantation embryos. In ETX embryos, lineage-specific stem cells bypass the blastocyst structure to directly assemble a post-implantation embryo.

FIG. 11 depicts development of a synthetic embryo formed from wild-type ES, TS, and XEN cells over time.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods of generating a synthetic embryo in vitro. In some embodiments, the method comprises: (a) providing a plurality of engineered embryonic stem cells (ESCs), wherein at least a portion of the plurality of engineered ESCs over-express E-cadherin (Cdh1); (b) providing a plurality of engineered trophoblast stem cells (TSCs), wherein at least a portion of the plurality of engineered TSCs over-express P-cadherin (Cdh3); (c) providing a plurality of extra-embryonic (XEN) cells; and (d) contacting the plurality of engineered ESCs, the plurality of engineered TSCs and the plurality of extra-embryonic (XEN) cells with a first culture media to form a co-culture; wherein the plurality of engineered ESCs and derivatives thereof, the plurality of engineered TSCs and derivatives thereof, and the plurality of XEN cells and derivatives thereof organize to form a synthetic embryo, wherein the synthetic embryo comprises one TS-derived compartment and one ES-derived compartment, and is covered by an outside XEN-derived monolayer.

Disclosed herein include methods of investigating mechanisms involved in embryogenesis. Disclosed herein include methods of identifying a compound useful for treating a disease. In some embodiments, the method comprises contacting the synthetic embryo or a differentiated cell(s) obtainable by any of the methods provided herein with the compound. Disclosed herein include methods for diagnosing or treating a disease or disorder in a subject. In some embodiments, the method comprises use of a synthetic embryo, or a differentiated cell(s) obtainable by the methods provided herein, or any combination thereof. Also provided are methods of elucidating the role of a gene in embryo development. In some embodiments, the method comprises obtaining an ESC, a TSC, and/or a XEN cell where the gene has been modified or knocked out and culturing the cell to obtain a plurality of cells for use in the method for generating a synthetic embryo disclosed herein.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

The term “about,” as used herein when referring to a measurable value such as an amount of a compound, dose, time, temperature, and the like, is meant to encompass variations of 10%, 5%, 1%, 0.5%, or even 0.1% of a specified amount.

As used herein, the term “differentiation” can refer to the process by which an unspecialized (“uncommitted”) or less specialized cell acquires the features of a specialized cell such as, for example, a neuronal cell. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the lineage of a cell defines the heredity of the cell, i.e., which cells it came from and to what cells it can give rise. The lineage of a cell places the cell within a hereditary scheme of development and differentiation. As used herein, a “lineage-specific marker” can refer to a characteristic specifically associated with the phenotype of cells of a lineage of interest and can be used to assess the differentiation of an uncommitted cell to the lineage of interest.

As used herein, “markers”, “lineage markers” or, “lineage-specific markers” can refer to nucleic acid or polypeptide molecules that are differentially expressed in a cell of interest. Differential expression can mean an increased level for a positive marker and a decreased level for a negative marker as compared to an undifferentiated cell. The detectable level of the marker nucleic acid or polypeptide is sufficiently higher or lower in the cells of interest compared to other cells, such that the cell of interest can be identified and distinguished from other cells using any of a variety of methods known in the art. In some embodiments, a marker can be enriched. The term “enriched”, as used herein, shall have its ordinary meaning, and can also refer to a statistically significant increase in levels of a gene product (e.g., mRNA and/or protein) in one condition as compared to another condition (e.g., in one cell layer as compared to another cell layer).

The term, “concentration” as used herein shall have its ordinary meaning, and can also refer to (a) mass concentration, molar concentration, volume concentration, mass fraction, molar fraction or volume fraction, or (b) a ratio of the mass or volume of one component in a mixture or solution to the mass or volume of another component in the mixture or solution (e.g., ng/ml). In some embodiments, the concentration can refer to fraction of activity units per volume (e.g., U/ml).

The term “analogue” as used herein refers to a compound which may be structurally related to the relevant molecule. The term “agonist” as used herein can refer to a compound which might not be structurally related to the relevant molecule. For example, an agonist may activate the relevant receptor by altering the conformation of the receptor. Nevertheless, in both cases the terms are used in this specification to refer to compounds or molecules which can mimic, reproduce or otherwise generally substitute for the specific biological activity of the relevant molecule.

As used herein the terms “culture medium” and “media” can be used interchangeable, and can refer to a liquid substance used to support the growth and development of stem cells and of an embryo. The culture medium used according to some embodiments of the invention can be a water-based medium which includes a combination of substances such as salts, nutrients, minerals, vitamins, amino acids, nucleic acids, and/or proteins such as cytokines, growth factors and hormones needed for cell growth and embryo development.

### Methods for Generating Synthetic Embryos

Previous systems to assemble synthetic embryos from multiple stem cell types are inefficient. Only about 10-15% of the synthetic embryos that self-organize from the 3 constituent stem cell lines (ES, TS and XEN/or induced XEN through Gata4/6 or Sox17 induction in ES) have the correct topology of the 5 day old natural embryo: distinct EPI and TE compartments completely surrounded by VE. The mechanisms that underlie this success rate are of interest to, e.g., optimize the efficiency of correct synthetic embryo assembly and also to discover the basic principles of embryo self-assembly, and how they go awry in synthetic and natural embryos.

As disclosed herein, differential cadherin expression as well as differential tension among different types of stem cells can be exploited to optimize the efficiency of self-organization. Through either the regulated expression of adhesion and tension regulatory molecules, or of transcription factors that define stem cell identity, the efficiency of synthetic embryo formation can be increased from 10-15% to 35-45%. The present disclosure reveals genetic and biophysical principles underlying proper formation of synthetic post-implantation embryos, in preparation for gastrulation and neurulation. In some embodiments, this strategy can be also applied to increase the formation efficiency of other types of organoids. A person skilled in the art will understand that several applications and uses of the disclosed methods are possible, with several non-limiting examples provided below.

Without being bound by any particular theory, assembly of the embryo is regulated by instructions that are intrinsic to the embryo (e.g., “self-assembly”). As described herein the self-organization of many cellular systems relies on the formation of distinct cell-cell contacts, which in turn depend on differential adhesion, via cadherin molecules and/or differential cortical tension, due to reorganization of the actin cortical network. Indeed, synthetic genetic programs controlling the expression of distinct cadherins can direct the formation of custom multicellular structures that have illuminated fundamental principles of self-organization. However, the principles that operate in the self-assembly of in vitro embryo structures have never been examined.

In some embodiments, given self-organization is the major bottleneck to efficient multi-cell-type embryo and organoid assembly, the application of knowledge (e.g., using the methods disclosed herein) about this stage to build more robust embryo models of early development or different types of organoids offers the potential to transform research in regenerative medicine and drug discovery, especially in generating stem cell models for early human development.

Furthermore, as an improvement from previous screens in organoids, the stem cell embryo methodologies provided herein can be applied toward multiple translational goals, including, but not limited to, improving the efficiency of generation of other types of organoids and in identifying genetic and pharmacological modulators of regulative mechanisms of embryo and organ development.

In some embodiments, a comprehensive understanding of the mechanisms of cell sorting, tissue segregation and morphogenesis at this critical developmental stage can shed light on the pathological alterations that lead to embryo lethality and congenital malformations. In addition, many of these processes are disrupted in diseases such as cancer and fibrosis, therefore the cellular mechanisms uncovered using the disclosed methods can be relevant to disease biology.

In some embodiments, identifying principles of self-organization in mammalian natural embryos and stem-cell embryos will help unveil regulatory pathways of natural development that allow mammalian embryos to “correct mistakes”, which will have significant translational value.

In some embodiments, knowledge gained from studying mammalian (e.g., mouse) stem cell embryo models can lead to successful construction of corresponding human stem cell embryo models by regulating differential cadherin expression and cortical tension among different cell types. Such an experimental model will be invaluable in the study of the mechanisms associated with early human development and disease. The tractable nature of these in vitro model systems will allow the detailed exploration of many aspects of human embryogenesis.

Disclosed herein include methods of generating a synthetic embryo in vitro. In some embodiments, the method comprises: (a) providing a plurality of engineered embryonic stem cells (ESCs), wherein at least a portion of the plurality of engineered ESCs over-express E-cadherin (Cdh1); (b) providing a plurality of engineered trophoblast stem cells (TSCs), wherein at least a portion of the plurality of engineered TSCs over-express P-cadherin (Cdh3); (c) providing a plurality of extra-embryonic (XEN) cells; and (d) contacting the plurality of engineered ESCs, the plurality of engineered TSCs and the plurality of extra-embryonic (XEN) cells with a first culture media to form a co-culture; wherein the plurality of engineered ESCs and derivatives thereof, the plurality of engineered TSCs and derivatives thereof, and the plurality of XEN cells and derivatives thereof organize to form a synthetic embryo, wherein the synthetic embryo comprises one TS-derived compartment and one ES-derived compartment, and is covered by an outside XEN-derived monolayer.

### Cadherins

Cadherins (named for “calcium-dependent adhesion”) are cell adhesion molecules important in forming adherens junctions that let cells adhere to each other. Cadherins are a class of type-1 transmembrane proteins, and they depend on calcium (Ca²⁺) ions to function, hence their name. Cell-cell adhesion is mediated by extracellular cadherin domains, whereas the intracellular cytoplasmic tail associates with numerous adaptors and signaling proteins. The cadherin superfamily includes the classical cadherins, desmogleins, desmocollins, protocadherins and more, and these proteins function in multiple processes including, but not limited to, cell adhesion and morphogenesis.

Cdh1 gene encodes E-cadherin, a classical cadherin of the cadherin superfamily. Alternative splicing results in multiple transcript variants, at least one of which encodes a preproprotein that is proteolytically processed to generate the mature glycoprotein. This calcium-dependent cell-cell adhesion protein is comprised of five extracellular cadherin repeats, a transmembrane region and a highly conserved cytoplasmic tail. Mutations in this gene are correlated with gastric, breast, colorectal, thyroid, and ovarian cancer. Loss of function of this gene is thought to contribute to cancer progression by increasing proliferation, invasion, and/or metastasis. The ectodomain of this protein mediates bacterial adhesion to mammalian cells and the cytoplasmic domain is required for internalization. This gene is present in a gene cluster with other members of the cadherin family on chromosome 16. The NCBI Gene is 999, Ensemble ID is ENSG00000039068, and UniProt ID is P12830.

Cdh3 gene encodes P-cadherin, a classical cadherin of the cadherin superfamily. Alternative splicing results in multiple transcript variants, at least one of which encodes a preproprotein that is proteolytically processed to generate the mature glycoprotein. This calcium-dependent cell-cell adhesion protein is comprised of five extracellular cadherin repeats, a transmembrane region and a highly conserved cytoplasmic tail. This gene is located in a gene cluster in a region on the long arm of chromosome 16 that is involved in loss of heterozygosity events in breast and prostate cancer. In addition, aberrant expression of this protein is observed in cervical adenocarcinomas. Mutations in this gene are associated with hypotrichosis with juvenile macular dystrophy and ectodermal dysplasia, ectrodactyly, and macular dystrophy syndrome (EEMS). The NCBI gene is 1001. The Ensemble ID is ENSG00000062038, and the UniProt ID is P22223.

Cdh6 gene encodes K-cadherin, a type II cadherin that may play a role in kidney development as well as endometrium and placenta formation. Decreased expression of this gene may be associated with tumor growth and metastasis. The NCBI gene is 1004. The Ensemble ID is ENSG00000113361), and the UniProt ID is P55285.

### Stem Cells, Embryogenesis and Mammalian Development

In some embodiments, the methods disclosed herein do not comprise any in vivo step. In some embodiments, none of ESCs, the TSCs, the XEN cells, the multicellular aggregate or synthetic embryo is present in an in vivo environment in any of the culturing steps disclosed herein. The in vivo environment can comprise a tissue, an organ, an organism, or a combination thereof.

Disclosed herein include methods of generating a synthetic embryo in vitro. In some embodiments, the method comprises: (a) providing a plurality of engineered embryonic stem cells (ESCs), wherein at least a portion of the plurality of engineered ESCs over-express E-cadherin (Cdh1); (b) providing a plurality of engineered trophoblast stem cells (TSCs), wherein at least a portion of the plurality of engineered TSCs over-express P-cadherin (Cdh3); (c) providing a plurality of extra-embryonic (XEN) cells; and (d) contacting the plurality of engineered ESCs, the plurality of engineered TSCs and the plurality of extra-embryonic (XEN) cells with a first culture media to form a co-culture; wherein the plurality of engineered ESCs and derivatives thereof, the plurality of engineered TSCs and derivatives thereof, and the plurality of XEN cells and derivatives thereof organize to form a synthetic embryo, wherein the synthetic embryo comprises one TS-derived compartment and one ES-derived compartment, and is covered by an outside XEN-derived monolayer. The method can comprise (e) replacing the first culture media with a second culture media about three days after the contacting step (d).

While mammalian embryogenesis has some common features across all species, it will be appreciated that different mammalian species develop in different ways and at different rates. In general, though, the fertilized egg undergoes a number of cleavage steps (passing through two cell, four cell and eight cell stages) before undergoing compaction to form a solid ball of cells called a morula, in which the cells continue to divide. Ultimately the internal cells of the morula give rise to the inner cell mass and the outer cells to the trophectoderm. The morula in turn develops into the blastocyst, which is surrounded by trophectoderm and contains a fluid-filled vesicle, with the inner cell mass at one end.

The term “embryo” as used herein refers to a mammalian organism from the single cell stage. The embryo described herein is generated from culturing in vitro from stem cells under appropriate conditions and resembles a natural embryo produced in vivo of a corresponding stage, such as having similar morphology, length, weight, cell type compositions and expression of developmental marker genes. A developmental stage of an embryo can be defined by the development of specific structures and can be used to define equivalent stages in development of other species. In some embodiments, a developmental stage of an embryo can be defined according to “Carnegie stages”, which is a standardized system used to provide a unified developmental chronology of the vertebrate embryo.

TABLE 1
CARNEGIE STAGES

| | Days since | |
|---|---|---|
| Carnegie | ovulation | |
| stage | (approx.) | Characteristic events/structures |
| 1 | 1 | fertilization; polar bodies |
| 2 | 2-3 | cleavage; morula; compaction |
| 3 | 4-5 | blastocyst and blastocoele; trophoblast and embryoblast |
| 4 | 6 | syncytiotrophoblast; cytotrophoblast; anchoring to endometrium |
| 5(a) | 7-8 | implantation; embryonic disc; bilaminar germ disc; primary yolk sac; |
| 5(b) | 9-10 | formation of trophoblast lacunae; complete penetration into |
| | | endometrium; amniotic cavity; primary umbilical vesicle |
| 5(c) | 11-16 | pre-chordal plate; extra-embryonic mesoblast; secondary yolk sac |
| 6 | 17 | Primitive streak, primitive node, primitive groove; secondary umbilical |
| | | vesicle; primordial germ cells; body stalk; early gastrulation. |
| 7 | 19 | Gastrulation; neural plate; start of hematopoiesis. |
| 8 | 23 | Primitive pit |
| 9 | 25 | Neural groove; neural folds; septum transversum; placode; early heart |

In some embodiments, the mammalian embryos generated herein are mouse embryos. Theiler has established numbered stages of murine development. The earliest stages, as applied to (C57BLxCBA)F1 mice, are described in the “emouse digital atlas” (www.emouseatlas.org) as follows in Table 2.

TABLE 2
THEILER STAGES

| Theiler | | | | |
|---|---|---|---|---|
| Stage | Dpc* (range) | | Cell number | (C57BL × CBA)Fl mice |
| 1 | 0-0.9 | (0-2.5) | 1 | One-cell egg |
| 2 | 1 | (1-2.5) | 2-4 | Dividing egg |
| 3 | 2 | (1-3.5) | 4-16 (or 8-16) | Morula |
| 4 | 3 | (2-4) | 16-40 (or 16-32) | Blastocyst inner cell mass apparent |
| 5 | 4 | (3-5.5) | | Blastocyst (zona-free) |
| 6 | 4.5 | (4-5.5) | | Attachment of blastocyst, primary endoderm covers |
| | | | | blastocoelic surface of inner cell mass |
| 7 | 5 | (4.5-6) | | Implantation and formation of egg cylinder, |
| | | | | Ectoplacental cone appears, enlarged epiblast, |
| | | | | primary endoderm lines mural trophectoderm |
| 8 | 6 | (5-6.5) | | Differentiation of egg cylinder, Implantation sites |
| | | | | 2 × 3 mm. Ectoplacental cone region invaded by |
| | | | | maternal blood, Reichert's membrane and proamniotic |
| | | | | cavity form |
| 9a | 6.5 | (6.25-7.25) | | Pre-streak(PS), advanced endometrial reaction, ecto |
| | | | | lacental cone invaded by blood, |
| | | | | extraembryonic ectoderm, embryonic axis visible |
| 9b | | | | Early streak(ES), gastrulation starts, first evidence |
| | | | | of mesoderm |
| 10a | 7 | (6.5-7.75) | | Mid streak (MS), amniotic fold starts to form |
| 10b | | | | Late streak, no bud (LSOB), exocoelom |
| 10c | | | | Late streak, early bud (LSEB), allantoic bud first |
| | | | | appears, node, amnion closing |
| 11a | 7.5 | (7.25-8) | | Neural plate (NP), head process developing, amnion |
| | | | | complete |
| 11b | | | | Late neural plate (LNP), elongated allantoic bud |
| 11c | | | | Early head fold (EDF) |
| 11d | | | | Late head fold (LHF), foregut invagination |
| 12a | 8 | (7.5-8.75) | | 1-4 somites, allantois extends, first branchial arch, |
| | | | | heart starts to form, foregut pocket visible, preotic |
| | | | | sulcus at 2-3 somite stage) |
| 12b | | | | 5-7 somites, allantois contacts chorion at the end of |
| | | | | TS12, Absent 2^{nd} arch, >7 somites |
| 13 | 8.5 | (8-9.25) | | Turning of the embryo, 1^{st} branchial arch has |
| | | | | maxillary and mandibular components, 2^{nd} arch |
| | | | | present; Absent 3rd branchial arch |
| 14 | 9 | (8.5-9.75) | | Formation & closure of ant. neuropore, otic pit |
| | | | | indented but not closed, 3^{rd} branchial arch visible; |
| | | | | Absent forelimb bud |
| 15 | 9.5 | (9-10.5) | | Formation of post. neuropore, forelimb bud, |
| | | | | forebrain vesicle subdivides; Absent hindlimb bud, |
| | | | | Rathke's pouch |
| 16 | 10 | (9.5-10.75) | | Posterior neuropore closes, Formation of hindlimb |
| | | | | & tail buds, lens plate, Rathke's pouch; the indented |
| | | | | nasal processes start to form. Absent thin & long tail |
| 17 | 10.5 | (10-11.25) | | Deep lens indentation, adv. < level of brain tube, tail |
| | | | | elongates and thins, umbilical hernia starts to form; |
| | | | | Absent nasal pits |
| 18 | 11 | (10.5-11.25) | | Closure of lens vesicle, nasal pits, cervical somites |
| | | | | no longer visible; Absent auditory hillocks, anterior |
| | | | | footplate |
| 19 | 11.5 | (11-12.25) | | Lens vesicle completely separated from the surface |
| | | | | epithelium, Anterior, but no posterior, footplate. |
| | | | | Auditory hillocks first visible; Absent retinal |
| | | | | pigmentation and sign of fingers |
| 20 | 12 | (11.5-13) | | Earliest sign of fingers, (splayed out), posterior |
| | | | | footplate apparent, retina pigmentation apparent, |
| | | | | tongue well-defined, brain vesicles clear; Absent 5 |
| | | | | rows of whiskers, indented |
| 21 | 13 | (12.5-14) | | Anterior footplate indented, elbow and wrist |
| | | | | identifiable, 5 rows of whiskers, umbilical hernia now |
| | | | | clearly apparent; Absent hair follicles, fingers |
| | | | | separate distally |
| 22 | 14 | (13.5-15) | | Fingers separate distally, only indentations between |
| | | | | digits of the posterior footplate, long bones of limbs |
| | | | | present, hair follicles in pectoral, pelvic and trunk |
| | | | | regions; Absent open eyelids, hair follicles in |
| | | | | cephalic region |

| 23 | 15 | Fingers & Toes separate, hair follicles also in |
|---|---|---|
| | | cephalic region but not at periphery of vibrissae, |
| | | eyelids open; Absent nail primordia, fingers 2-5 |
| | | parallel |
| 24 | 16 | Reposition of umbilical hernia, eyelids closing, |
| | | fingers 2-5 are parallel, nail primordia visible on Toes; |
| | | Absent wrinkled skin, fingers & toes joined |
| | | together |
| 25 | 17 | Skin is wrinkled, eyelids are closed, umbilical hernia |
| | | is gone; Absent ear extending over auditory meatus, |
| | | long whiskers |
| 26 | 18 | Long whiskers, eyes barely visible through closed |
| | | eyelids, ear covers auditory meatus |
| 27 | 19 | Newborn Mouse |

*“dpc” indicates days post conception, with the morning after the vaginal plug is found being designated 0.5 d or E0.5.

The developmental stage of a synthetic embryo generated herein can be defined according to its embryonic day. As used herein, the term “embryonic day (E)” in the context of a mammalian embryo (e.g., mouse embryo) refers to an embryo having developmental characteristic of an in vivo (in-uterine tube or in utero) mammalian embryo counterpart at the specified day following fertilization, wherein EO is considered as the fertilized egg.

In some embodiments, the methods and compositions described herein enable culture up to or through to stages corresponding to Theiler stage 7, 8, 9(a), 9(b), 10(a), 10(b), 10(c), 11(a), 11(b), 11(c), 11(d), 12(a), 12(b), 13, 14, 15, 16 and beyond, Carnegie stage (a), 5(b), 5(c), 6, 7, 8, 9 and beyond, and corresponding stages in other species (e.g. post-implantation stages). In some embodiments, the synthetic embryo generated herein can reach stages of at least E4, E4.5, E5, E5.5, E6, E6.5, and beyond (e.g., post-implantation stages).

The methods and compositions herein described can be applied to embryos from any suitable mammalian species, such as: primates, including humans, great apes (e.g., gorillas, chimpanzees, orangutans), old world monkeys, new world monkeys; rodents (e.g., mice, rats, guinea, pigs, hamsters); cats; dogs; lagomorphs (e.g., rabbits); cows; sheep; goats; horses; pigs; and any other livestock, agricultural, laboratory or domestic mammals. The methods and compositions herein described can be applied to an embryo from any non-human mammal, including but not limited to those described above. Thus, any of the culture media embodiments defined herein may be capable of supporting development of a nonhuman mammalian embryo on a substrate from a pre-implantation stage of development to a post-implantation stage of development.

The term “pre-implantation stage” can be used herein to refer to a stage of development earlier than the stage corresponding to Theiler stage 7, Carnegie stage 5(a), and corresponding stages in other species. As used herein, the term “post-implantation stage” can refer to a stage of development later than the stage corresponding to Theiler stage 7, Carnegie stage 5(a), and corresponding stages in other species. A “post-implantation stage” may be determined by detecting the up-regulation of one or more genes by the embryo.

The term “stem cell,” as used herein, refers to a cell that is capable of differentiating into one or more differentiated cell types. Stem cells may be totipotent. Stem cells may be pluripotent cells. Totipotent stem cells typically have the capacity to develop into any cell type. Totipotent stem cells are usually embryonic in origin. The term “progenitor cell,” as used herein, refers to a cell that is committed to a particular cell lineage and which gives rise to a particular limited range of differentiated cell types by a series of cell divisions. An example of a progenitor cell would be a myoblast, which is capable of differentiation to only one type of cell, but is itself not fully mature or fully differentiated.

Examples of stem cells include: embryonic stem (ES) cells with pluripotency derived from a fertilized egg or clone embryo; epiblast stem cells; trophoblast stem cells; extraembryonic endoderm (XEN) stem cells; somatic stem cells and pluripotent stem cells that are present in tissues in a living organism e.g. hepatic stem cells, dermal stem cells, and reproductive stem cells that serve as the bases for respective tissues; pluripotent stem cells derived from reproductive stem cells; pluripotent stem cells obtained by nuclear reprogrammed somatic cells; totipotent stem cells and non-totipotent stem cells and the like. Also, partially committed stem cells e.g. progenitor cells may be cultured using the media and according to the methods described herein.

The methods and compositions described herein may be applied to stem cells from any suitable mammalian species, such as: primates, including humans, great apes (e.g., gorillas, chimpanzees, orangutans), old world monkeys, new world monkeys; rodents (e.g., mice, rats, guinea pigs, hamsters); cats; dogs; lagomorphs (e.g., rabbits); cows; sheep; goats; horses; pigs; and any other livestock, agricultural, laboratory or domestic mammals. The methods and compositions described herein may be applied to stem cells from any non-human mammal, including but not limited to those described above. In some embodiments, the non-human mammals are rodents.

The term “pluripotent stem cell” (PSC) as used herein can refer to cells that are capable of differentiating into several different, final differentiated cell types. Pluripotent stem cells can originate from various tissue or organ systems, including, but not limited to, blood, nerve, cardiac and skeletal muscle, skin, gut, bone, kidney, liver, pancreas, thymus, and the like. In some embodiments, PSCs can be cultured in vitro and have the potential for differentiating into all cells, but the placenta. The pluripotent stem cell has the potential to differentiate into any of the three germ layers: endoderm (which forms structures such as the gastrointestinal tract and the respiratory system), mesoderm (which forms structures such as the musculoskeletal system, the vascular system and the urogenital system), or ectoderm (which forms epidermal tissues and the nervous system).

A PSC may be obtained from a fertilized egg, clone embryo, reproductive stem cell, or stem cell in tissue. Also included are cells having differentiation pluripotency similar to that of embryonic stem cells, conferred artificially by transferring several different genes to a somatic cell (also referred to as induced pluripotent stem cells or iPS cells). Induced pluripotent stem cells may be derived from any suitable source (e.g. hair follicles, skin cells, fibroblasts, etc.). Pluripotent stem cells can be prepared by known methods in the art. Any of the stem cells as defined herein may be derived from diseased or non-diseased tissue. Stem cells can be from any suitable mammalian species, such as: primates, including humans, great apes (e.g. gorillas, chimpanzees, orangutans), old world monkeys, new world monkeys; rodents (e.g. mice, rats, guinea pigs, hamsters); cats; dogs; lagomorphs (including rabbits); cows; sheep; goats; horses; pigs; and any other livestock, agricultural, laboratory or domestic mammals. The presently disclosed methods may be applied to stem cells from any non-human mammal, including but not limited to those described above.

In some embodiments, the PSC cells disclosed herein are mammalian embryonic stem cells (ESCs). The term “embryonic stem cell” (ES cell) as used herein refers to a pluripotent stem cell derived from the inner cell mass of a blastocyst, which is an early-stage preimplantation embryo. It is envisaged that such cells may express genes involved in the naive pluripotency network (Oct4/Nanog, Sox2, Klf4 etc.). Such cells may also have Oct4 proximal enhancer activity. They may contribute to all embryonic tissues in chimeras. The ES cells may be derived from mammalian embryos, obtained from iPS cells or obtained from appropriate cell lines. Non-limiting examples of said stem cells include embryonic stem cells of a mammal or the like established by culturing a pre-implantation early embryo, embryonic stem cells established by culturing an early embryo prepared by nuclear-transplanting the nucleus of a somatic cell, induced pluripotent stem cells (iPS cells) established by transferring several different transcriptional factors to a somatic cell, and pluripotent stem cells prepared by modifying a gene on a chromosome of embryonic stem cells or iPS cells using a gene engineering technique. More specifically, embryonic stem cells include embryonic stem cells established from an inner cell mass that constitutes an early embryo, embryonic stem cells established from a primordial germ cell, cells isolated from a cell population possessing the pluripotency of pre-implantation early embryos (for example, primordial ectoderm), and cells obtained by culturing these cells.

As would be understood by a person of skill in the art, ES cells may be obtained from stem cell banks such as the UK stem cell bank from which human stem cell lines for research can be acquired. The Jackson Laboratory, US (who provide Jax mice) also stores and derives mouse ES cells which are available for purchase. It is preferred that the ES cells are obtained or are obtainable by a method that does not involve the destruction of human or non-human animal embryos.

The term “trophoblast stem cell” (TS) as used herein refers to stem cells derived from the trophoblast lineage of the embryo. The trophoblast stem cells are preferably not extra-embryonic cells derived from the two cell types which are precursors of the human placenta: the cytotrophoblast and the syncitiotrophoblast. These cells can be derived at late pre-implantation stages E4.5 or early post-implantation stages (E5.5) but the resulting cell lines are equivalent to the stem cell compartment existing in the extra-embryonic ectoderm of the post-implantation mouse egg cylinder. Transcription factors such as Elf5, Eomes, and Tfap2C mark this lineage. TS cells can also be considered as cells that are the precursors of the differentiated cells of the placenta. In the mouse, TS cells can be derived from outgrowths of either blastocyst polar trophectoderm or extraembryonic ectoderm, which originates from polar trophectoderm after implantation.

The term “extra-embryonic endoderm stem cell” (XEN stem cell) as used herein refers to stem cells derived from the extraembryonic endoderm of an embryo (e.g., a mouse embryo). The extraembryonic endoderm is typically a derivative of the hypoblast cells that migrate into the blastocyst cavity (beginning on day 8 of human embryonic development), and line the cavity, giving rise to the primary and definitive yolk sacs. The extraembryonic endoderm fills the remaining cavity of the blastocyst. In some embodiments, the XEN stem cells used herein comprise an inducible XEN stem cell capable of expressing a GATA transcription factor upon induction (e.g., by doxycycline treatment). In some embodiments, the XEN cells also be induced from ESCs by overexpression of PrE-specific genes, GATA transcription factor (e.g., Gata4/6) or Sox J 7. or by treatment with growth factors. In some embodiments, the XEN stem cells used herein are inducible XEN stem cells capable of expressing GATA4 upon induction.

### Synthetic Embryos

Disclosed herein include methods of generating a synthetic embryo in vitro. In some embodiments, the method comprises: (a) providing a plurality of engineered embryonic stem cells (ESCs), wherein at least a portion of the plurality of engineered ESCs over-express E-cadherin (Cdh1); (b) providing a plurality of engineered trophoblast stem cells (TSCs), wherein at least a portion of the plurality of engineered TSCs over-express P-cadherin (Cdh3); (c) providing a plurality of extra-embryonic (XEN) cells; and (d) contacting the plurality of engineered ESCs, the plurality of engineered TSCs and the plurality of extra-embryonic (XEN) cells with a first culture media to form a co-culture; wherein the plurality of engineered ESCs and derivatives thereof, the plurality of engineered TSCs and derivatives thereof, and the plurality of XEN cells and derivatives thereof organize to form a synthetic embryo, wherein the synthetic embryo comprises one TS-derived compartment and one ES-derived compartment, and is covered by an outside XEN-derived monolayer. The method can comprise (e) replacing the first culture media with a second culture media about three days after the contacting step (d). The method can comprise:(f) replacing the second culture media with a third culture media about one day after step (e).

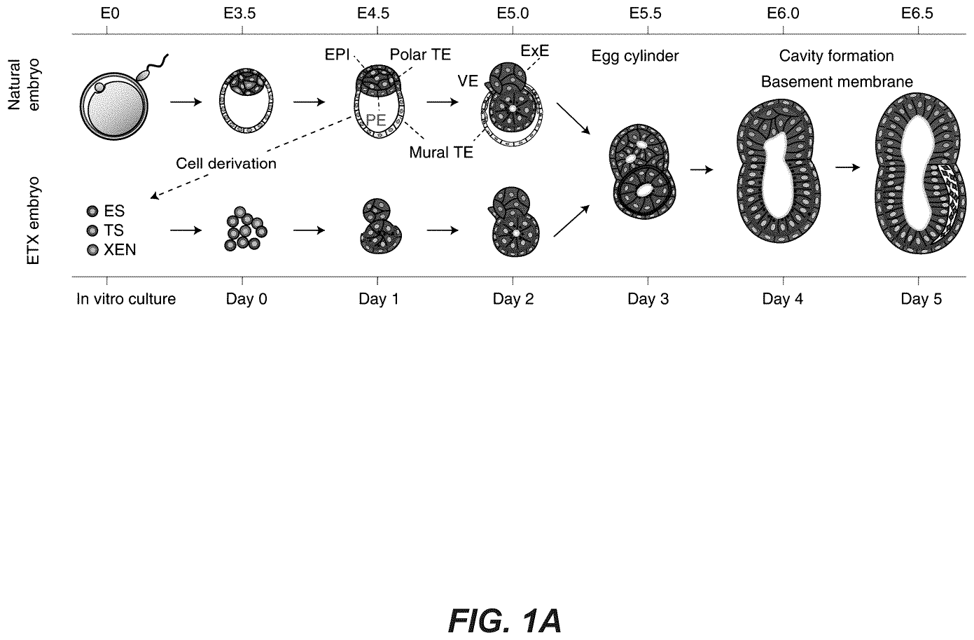

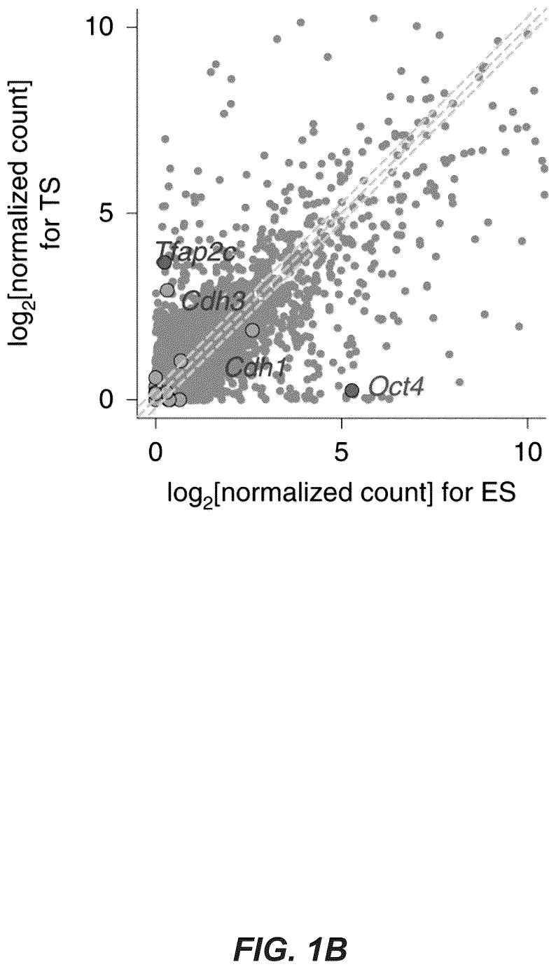

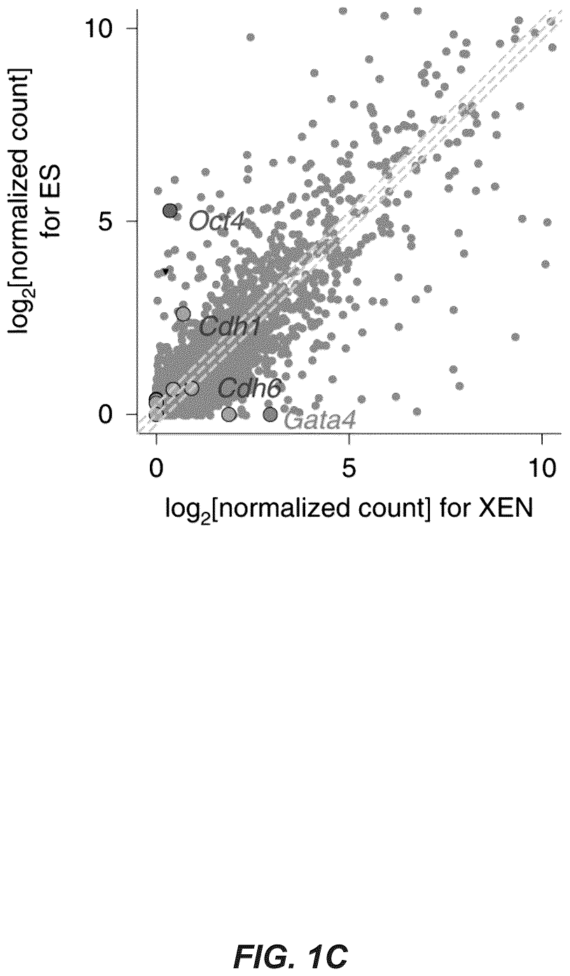

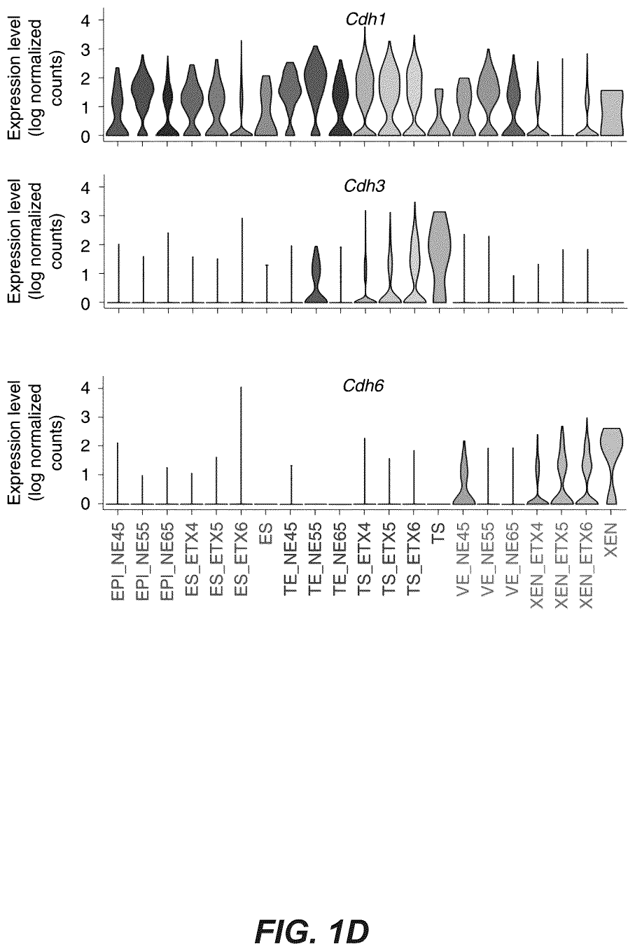

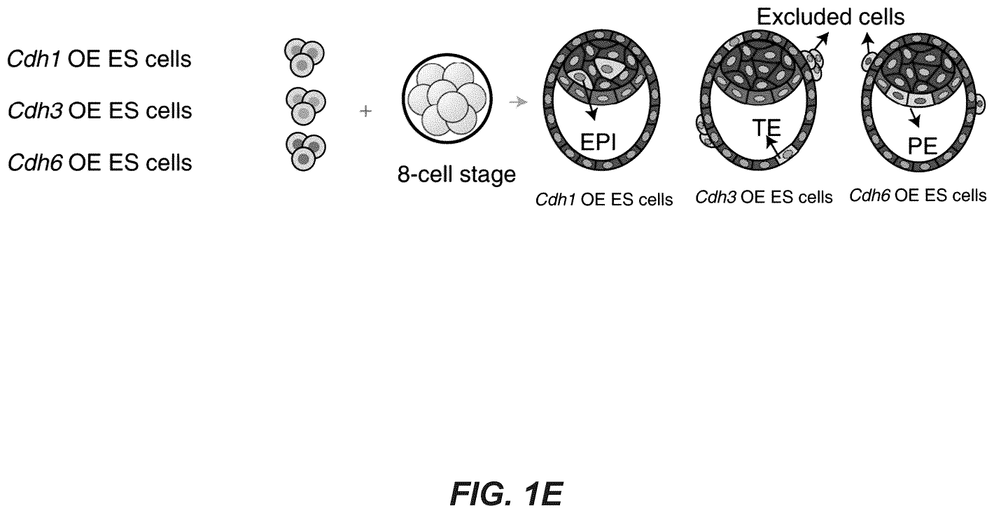

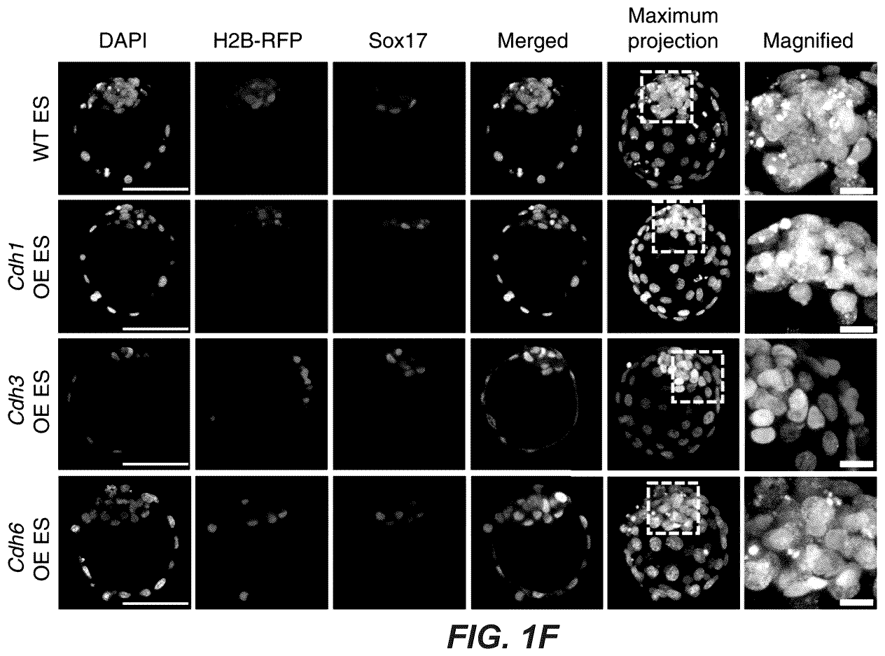

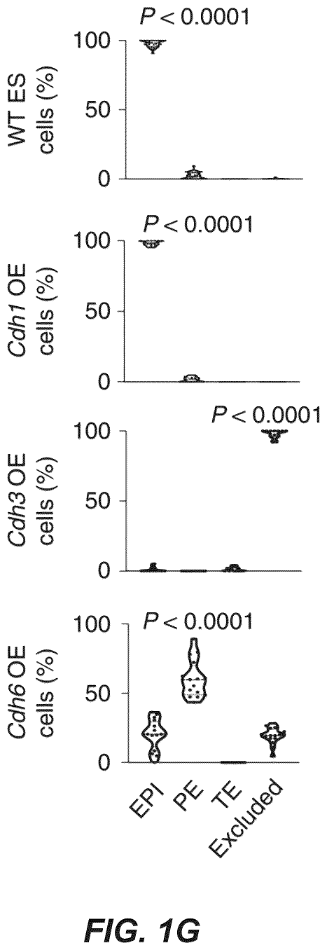

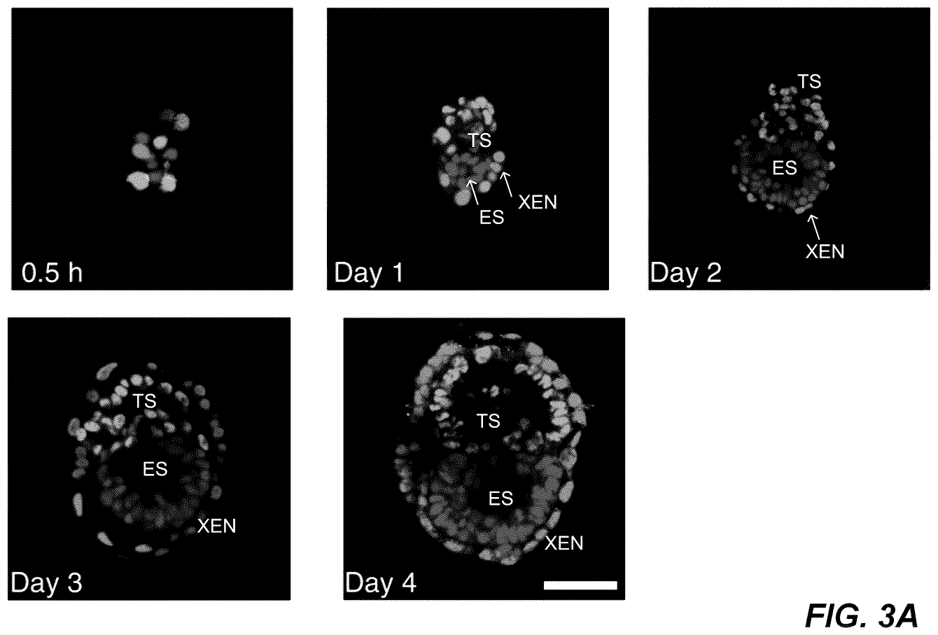

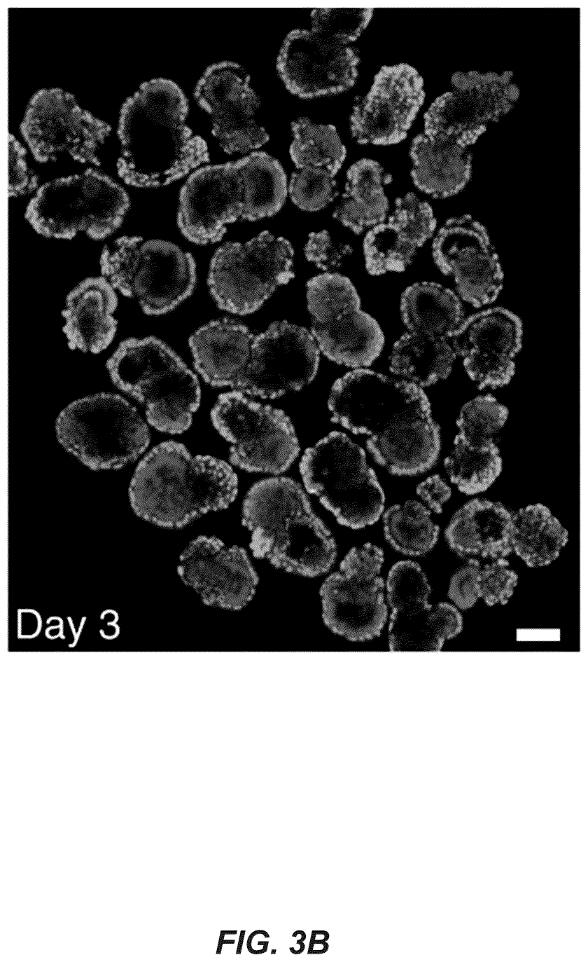

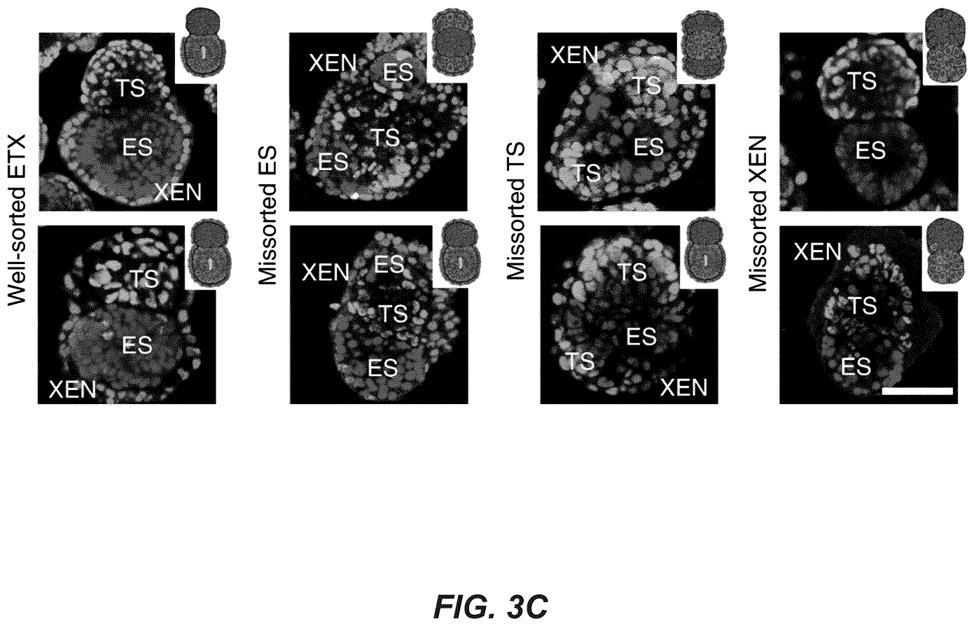

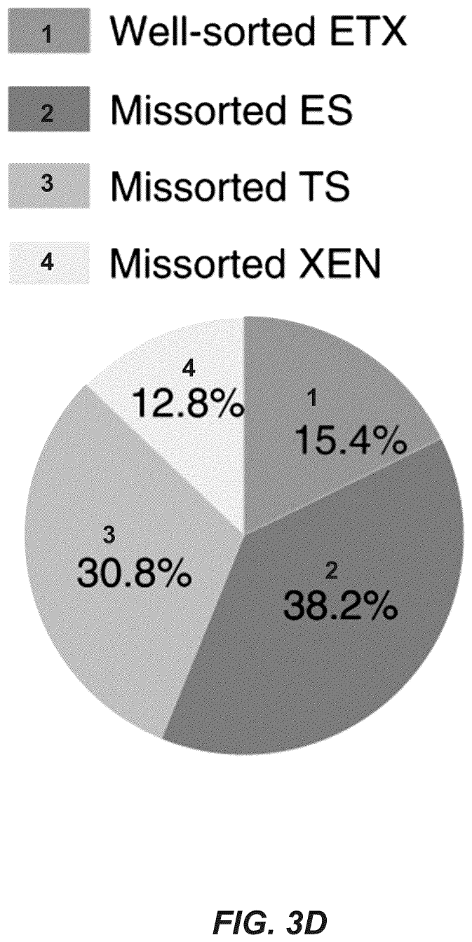

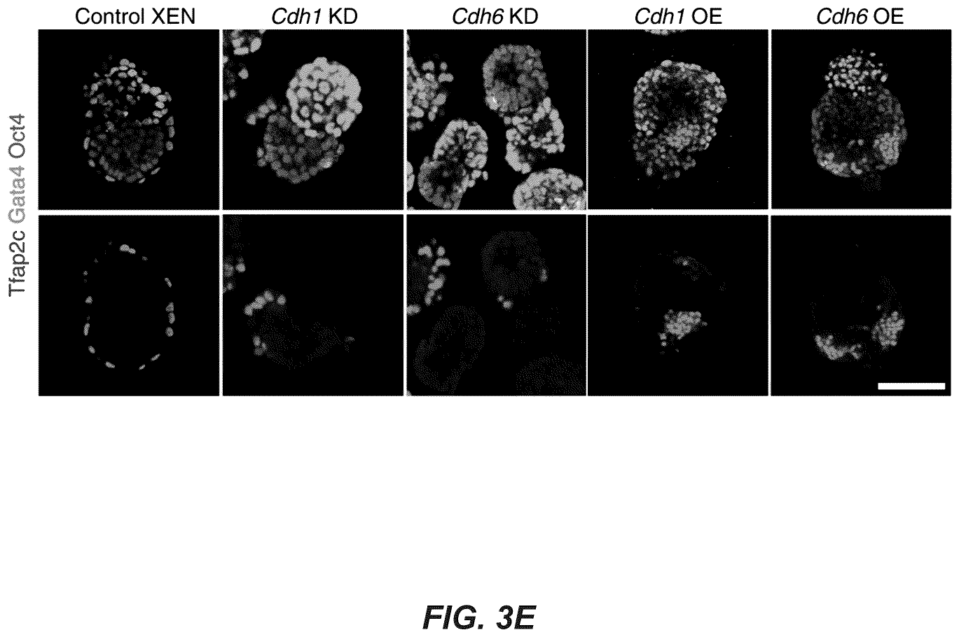

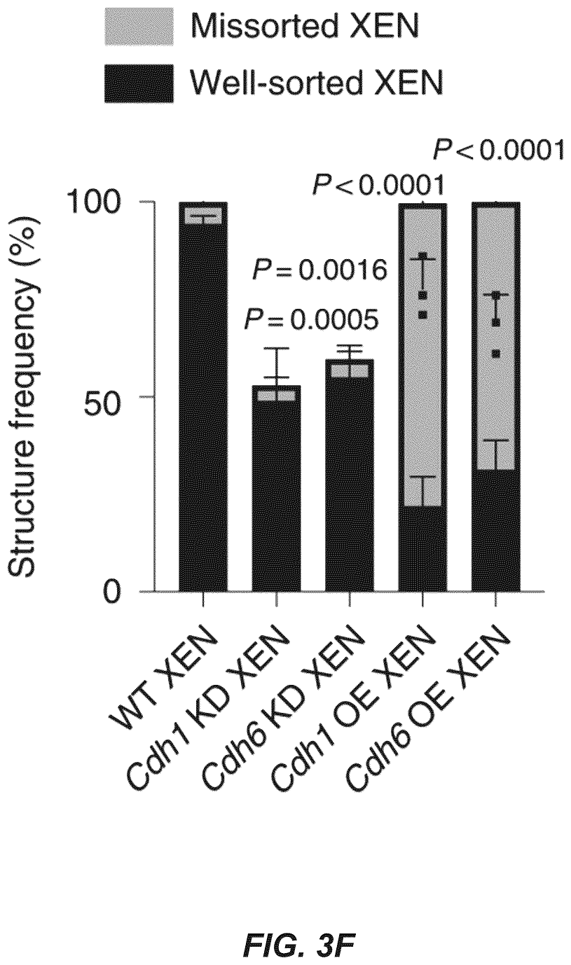

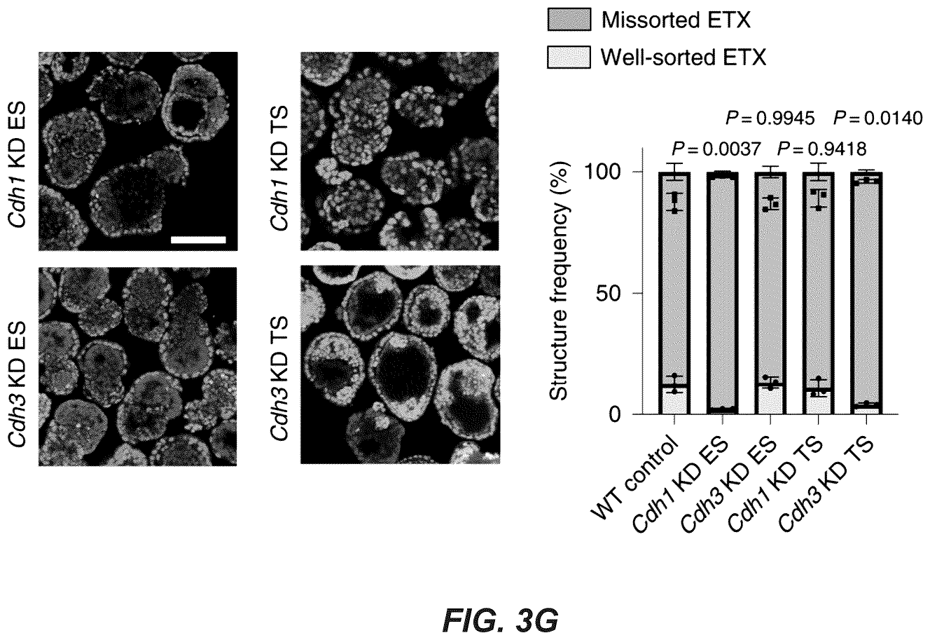

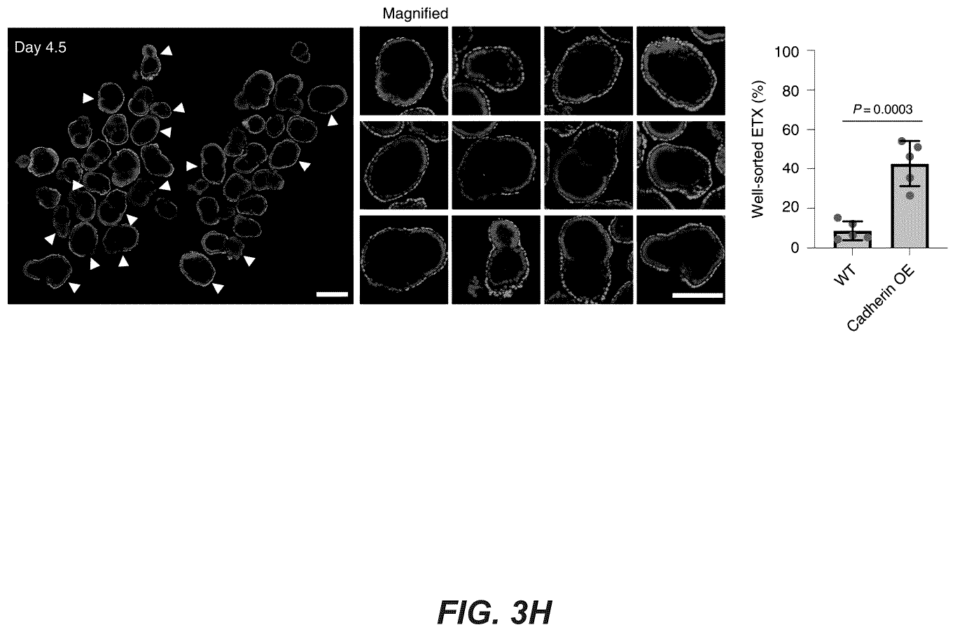

In some embodiments, the methods disclosed herein advantageously increase the probability and/or frequency of generating “well-sorted” synthetic embryos. As used herein, the term “well-sorted” shall be given its ordinary meaning and shall also refer to a multicellular aggregate or synthetic embryo that comprises one TS-derived compartment and one ES-derived compartment, partially or completely covered by an outside XEN-derived monolayer. Also, See, e.g., FIG. 1A and FIG. 3A.

In some embodiments, the plurality of engineered ESCs, the plurality of engineered TSCs, and the plurality of XEN cells organize into a multicellular aggregate structure within about 12-24 hours (e.g., about 12 hours, 13 hours, 14 hours, 15 hours, 16 hours, 17 hours, 18 hours, 19 hours, 20 hours, 21 hours, 22 hours, 23 hours, 1 day or a number or a range between any two of these values) of the contacting step. In some embodiments, the multicellular aggregate structure develops into a multicellular aggregate structure comprising one TS-derived compartment and one ES-derived compartment, at least partially covered by an outside XEN-derived monolayer (e.g., is “well-sorted”) with an efficiency of about 30% after about 12 hours following the contacting step. In some embodiments, the multicellular aggregate structure develops into a “well-sorted” multicellular aggregate structure with an efficiency of at least about 30% (e.g., 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) after about 12 hours following the contacting step. In some embodiments, the multicellular aggregate structure develops into the synthetic embryo (e.g., a “well-sorted” synthetic embryo) with an efficiency of about 40% after at least 3 days following the contacting step. In some embodiments, the multicellular aggregate structure develops into the synthetic embryo with an efficiency of at least about 40% (e.g., 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) after at least 3 days following the contacting step.

In some embodiments, the synthetic embryo (e.g., a “well-sorted” synthetic embryo) develops a single interior cavity with an efficiency of about 90%. In some embodiments, the synthetic embryo (e.g., a “well-sorted” synthetic embryo) develops a single interior cavity with an efficiency of at least about 90% (e.g., 90%, 91%, 92%, 93%, 94%, 95%, 96%, 97%, 98%, 99%, 100%, or a number or a range between any two of these values). In some embodiments, the single interior cavity develops between four and five days after the contacting step.

In some embodiments, the multicellular aggregate structure develops into the synthetic embryo (e.g., a “well-sorted” synthetic embryo) and comprises a single interior cavity, with an efficiency of about 40%. In some embodiments, the multicellular aggregate structure develops into the synthetic embryo (e.g., a “well-sorted” synthetic embryo) comprising a single interior cavity with an efficiency of least about 40% (e.g., 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values). In some embodiments, the single interior cavity develops between four and five days after the contacting of step.

In some embodiments, the multicellular aggregate structure develops into a synthetic embryo (e.g., a “well-sorted” synthetic embryo) comprising a laminin-containing basement membrane with an efficiency of about 78%. In some embodiments, the multicellular aggregate structure develops into a synthetic embryo (e.g., a “well-sorted” synthetic embryo) comprising a laminin-containing basement membrane with an efficiency of at least about 78% (e.g., 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% or a number or a range between any two of these values). In some embodiments, the laminin-containing basement membrane develops between four and five days after the contacting step.

In some embodiments, the synthetic embryo (e.g., a “well-sorted” synthetic embryo) has a length of at least 200 μm about 72 hrs following the contacting step. In some embodiments, the synthetic embryo (e.g., a “well-sorted” synthetic embryo) has a length of about 200 μm to about 500 μm (e.g., about 200 μm, 250 μm, 300 μm, 350 μm, 400 μm, 450 μm, 500 μm or a number or a range between any two of these values)about 72 hours following the contacting step. In some embodiments, the synthetic embryo has a size of at least 4×10³ μm² about 72 hrs following the contacting step. In some embodiments, the synthetic embryo (e.g., a “well-sorted” synthetic embryo) has a size of about 6×10³ μm² to about 10×10³ μm² (e.g., about 6×10³ μm², 6.5×10³ μm², 7×10³ μm², 7.5×10³ μm², 8×10³ μm², 8.5×10³ μm², 9×10³ μm², 9.5×10³ μm², 10×10³ μm² or a number or a range between any two of these values) about 72 hrs following the contacting step.

The TS-derived compartment can comprise cells that express at least one TS cell-marker. The at least one TS cell-marker can comprise Tfap2C, EOMES, or both. The ES-derived compartment can comprise cells that express at least one ES cell-marker. The at least one ES cell-marker can comprise Oct4. The XEN-derived monolayer can comprise cells that express at least one XEN cell-marker. The at least one XEN cell-marker can comprise Gata4, Gata6, or both. In some embodiments, the synthetic embryo resembles an egg cylinder structure, after about three days following the contacting step. In some embodiments, the synthetic embryo resembles a post-implantation embryo structure, after about four to five days following the contacting step (Also, See, Table 2).

Provided herein include methods, compositions, and culture medias for modeling mammalian embryo development by culturing stem cells including pluripotent stem cells (e g., embryonic stem cells) and extra-embryonic stem cells. The methods, compositions and culture media disclosed herein can generate synthetic embryos through various developmental stages. In some embodiments, the synthetic embryos generated using the methods and compositions described herein can reach a post-implantation (e.g., a post-implantation, pre-gastrulation stage). In some embodiments, the synthetic embryos generated herein can reach an early gastrulation stage. In some embodiments, the synthetic embryos generated herein can reach a late gastrulation stage. In some embodiments, the synthetic embryos generated herein can reach an early neurulation stage. In some embodiments, the synthetic embryos generated herein can reach a late neurulation stage. In some embodiments, the synthetic embryos generated herein can reach an early organogenesis stage or beyond.

The synthetic embryos generated using the methods and culture medias described herein can comprise post-implantation embryos, e.g., post-implantation pre-gastrulation embryo structure. As used herein, the term “post implantation pre gastrulation” in the context of a mammalian embryo (e.g., a mouse embryo) refers to an embryo following the implanting blastocyst stage and prior to the early gastrulation stage and is characterized by an egg cylinder-shape prior to symmetry breaking. An embryo of a post implantation pre gastrulation stage can be defined as Theiler stages TS7-TS8 (Also, See, Table 2). In some embodiments, the post-implantation, pre-gastrulation stage refers to E4.5-6.5, optionally, E4.5-6, optionally E5-6.5, optionally E5-5.5. In some embodiments, the post-implantation, pre-gastrulation stage refers to E5.5.

Embryonic stage of a synthetic embryo generated using the methods and culture medias disclosed herein can be assessed by comparing to an in vivo natural embryo counterpart at the same developmental stage by multiple ways including, but not limited to, morphology, length, weight, cell type compositions and expression of developmental marker genes (e.g., Oct4, Nanog, Sox2, Klf4, Cdx2, Gata4, Gata6, Brachyury, Otx2, Fgf5 and others described in the Examples and known in the art) using specific antibodies or primers, or transcriptional profiling, single-cell RNA sequencing and other methods as further described in the Examples section. In some embodiments, the post-implantation pre-gastrulation embryo structure resembles an E5.5 natural embryo structure. In some embodiments, the postimplantation pre-gastrulation embryo structure and an E5.5 natural embryo structure have similar morphology, cell type compositions, and gene expression features. In some embodiments, the post-implantation pre-gastrulation embryo structure comprises cavitated epithelial embryonic stem (ES) cell and trophoblast stem (TS) cell compartments enveloped by a VE-like layer.

In some embodiments, the synthetic embryo generated using the methods and culture medias described herein comprise post-implantation embryos, e. g., early gastrulation embryo structure. As used herein, the term “gastrulation” in the context of an embryo refers to an embryo following the expanded blastocyst stage and prior to the somitogenesis stage and is characterized by the formation of the primitive streak and epithelial to mesenchymal transition forming three germinal layers. The gastrulation process is generally considered as the process through which the bilaminar embryonic disc is changed into a trilaminar disc as an intraembryonic mesoderm appears between the ectoderm and endoderm. As used herein, the term “early gastrulation” in the context of a mammalian embryo (e.g., mouse embryo) refers to an embryo following the post-implantation pre-gastrulation stage and prior to a late gastrulation stage and is characterized by egg cylinder shape with the primitive streak at the posterior side. An embryo of an early gastrulation stage can be defined as Theiler stages TS8-TS10 (see Table 2). In some embodiments, the early gastrulation stage refers to E5-7.75, optionally E5-6.5, optionally E6.25-7.25, optionally 6.5-7.75, optionally E6.5-E7.5. In some embodiments, the early gastrulation stage refers to E6.5-E7.

In some embodiments, an early gastrulation embryo structure has a proamniotic cavity (resulting from the merger of cavities in the ES cell and TS cell compartments, a fully migrated AVE (as the boundary of the ES cell and TS cell compartments) and gastrulating. The gastrulation can be revealed by the epithelial-to-mesenchymal transition and formation of a cell layer between the ES cells and VE-like layers.

In some embodiments, culturing of a synthetic embryo from a postimplantation pre-gastrulation stage to an early gastrulation stage is achieved by culturing the embryo structure in a culture media for a suitable time period. In some embodiments, culturing of an embryo structure from a post-implantation pre-gastrulation stage to an early gastrulation stage is effected for at least one day (e.g., one, two or three days). In some embodiments, the culturing is from E5.5 to E6.6.

In some embodiments, the gastrulating embryo structures generated using the methods and culture medias described herein resemble natural gastrulating embryos. In some embodiments, the synthetic gastrulating embryos and natural gastrulating embryos have similar morphology, cell type compositions, and gene expression features. In some embodiments, culturing of a post-implantation pre-gastrulation embryo structure to its early gastrulation stage is continued allowing the post-implantation embryo structure to develop into a late gastrulation stage or to complete gastrulation. As used herein, the term “late gastrulation stage” in the context of a mammalian embryo (e.g., a mouse embryo) refers to an embryo following the early gastrulation stage and prior to the early somite stage and is characterized by an egg cylinder-shaped embryo with differentiated definitive endoderm, mesoderm and ectoderm layers. An embryo of a late gastrulation stage can be defined as Theiler stages TS10-TS11 (Also, See, Table 2). In some embodiments, a late gastrulation stage can correspond to E6.5-8, optionally E6.5-7.75, optionally E7.25-8, optionally E7-8.

In some embodiments, the synthetic embryos generated using the methods and culture conditions described herein are mammalian embryos. In some embodiments, the mammalian embryos are non-human embryos, such as mouse embryos or rabbit embryos. In some embodiments, the mammalian embryos are human embryos.

Embryonic stages of the synthetic embryos described herein can be assessed compared to an in vivo or natural embryo counterpart at the same developmental stage by multiple ways including, but not limited to, morphology, length, weight, weight, expression of developmental marker genes using specific antibodies or primers, transcriptional profiling and the like, as further described herein below and in the Examples section. Morphology assessment of embryonic development can be performed by previously established morphological features such as described in Carnegie stages of development (Also, See, Table 2; Developmental stages in human embryos. R. O'Rahilly and F. Muller (eds), Carnegie Institution of Washington, Washington, DC, 1987), in Theiler stages of development (see, for example, Table 2; www. emouseatlas org) or according to embryonic days.

In some embodiments, one or more developmental markers as described herein can be used to assess the developmental stage of a synthetic embryo structure. Numerous methods exist in the art for detecting the presence, absence, or amount of a marker gene product (e.g., mRNA and/or protein), as well as its localization in an embryo structure or subcellular localization (e.g., nucleus and/or cytoplasm). Marker expression may be assessed by any of a wide variety of well-known methods for detecting expression of a transcribed molecule or a protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification and sequencing methods.

In some embodiments, activity of a particular gene is characterized by a measure of gene transcript (e g., mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In another embodiment, detecting or determining expression levels of a marker and functionally similar homologs thereof, including a fragment or genetic alteration thereof (e.g., in regulatory or promoter regions thereof) comprises detecting or determining RNA levels for the marker of interest. In some embodiments, one or more cells from the synthetic embryo structure can be obtained and RNA is isolated from the cells. In some embodiments, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated. It is also be possible to obtain cells from, e.g., the synthetic embryo cells and culture the cells in vitro, such as to obtain a larger population of cells from which RNA can be extracted. Methods for establishing cultures of non-transformed cells, i.e., primary cell cultures, are known in the art. In some embodiments, cells can be dissociated (e.g., by enzymatic or mechanical means), and isolated by methods known in the art (e g., Fluorescence-Activated Cell Sorting, Microfluidics, etc.)

When isolating RNA from, e.g., synthetic embryos at various developmental stages and/or cells comprising said synthetic embryos, it may be important to prevent any further changes in gene expression after the tissue or cells has been removed from the subject. Changes in expression levels are known to change rapidly following perturbations, e g., heat shock or activation with lipopolysaccharide (LPS) or other reagents. In addition, the RNA in the tissue and cells may quickly become degraded. Accordingly, in some embodiments, the tissue or cells obtained from a subject is snap frozen as soon as possible.

RNA can be extracted from cells by a variety of methods, e.g., the guanidium thiocyanate lysis followed by CsCl centrifugation. Methods for obtaining RNA from single-cells are also known in the art. The RNA sample can then be enriched in particular species. In some embodiments, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.). In some embodiments, the RNA population is enriched in marker sequences. Enrichment can be undertaken, e.g., by primer-specific cDNA synthesis, or multiple rounds of linear amplification based on cDNA synthesis and template-directed in vitro transcription.

The population of RNA, enriched or not in particular species or sequences, can further be amplified. As defined herein, an “amplification process” increases the number of copies of a polynucleotide (e.g., RNA). For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the disclosed methods to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used. Other known amplification methods which can be utilized herein include but are not limited to the so-called “NASBA” or “3 SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e g., Kwoh et al., Proc. Natl. Acad. Sci. USA 86, 1173 (1989)). Many techniques are known in the state of the art for determining absolute and relative levels of gene expression, commonly used techniques suitable for use in the disclosed methods include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Nonradioactive labels such as digoxigenin may also be used. In some embodiments, the probe is labeled with a fluorescence moiety.

Alternatively, mRNA expression can be detected on a DNA array, chip or a microarray. Labeled nucleic acids of a test sample obtained from a subject may be hybridized to a solid surface comprising marker DNA. Positive hybridization signal is obtained with the sample containing marker transcripts. Methods of preparing DNA arrays and their use are well known in the art (see, e.g., U.S. Pat. Nos. 66,186,796; 6,379,897; 6,664,377; 6,451,536; 548,257; U.S. 20030157485). Serial Analysis of Gene Expression (SAGE) can also be performed (See for example U.S. Patent Application 20030215858). In some embodiments, next generation sequencing (e.g., RNA-seq) can be used to analyze total mRNA expression from one (e.g., single-cell RNA-seq) or more cells. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others. Methods for constructing sequencing libraries are known in the art.

The single cell sequencing can be high-throughput single cell RNA sequencing. In certain embodiments, the single cell sequencing is a low cost high-throughput single cell RNA sequencing. Not being bound by any particular theory, the single cell RNA sequencing is capable of efficiently and cost effectively sequencing thousands to tens of thousands of single cells. In certain embodiments, single cell RNA sequencing comprises pairing single cells in droplets with oligonucleotides for reverse transcription, wherein the oligonucleotides are configured to provide cell-of-origin specific barcodes uniquely identifying transcripts from each cell and a unique molecular identifier (UMI) uniquely identifying each transcript. In certain embodiments, single cell RNA sequencing comprises pairing single cells in droplets with single microparticle beads coated with oligonucleotides for reverse transcription, wherein the oligonucleotides contain a bead-specific barcode uniquely identifying each bead and a unique molecular identifier (UMI) uniquely identifying each primer. In some aspects of the disclosure, unbiased classifying of cells in a biological sample comprises sequencing the transcriptomes of thousands of cells, preferably tens of thousands of cells (e.g., greater than 1000 cells, or greater than 10,000 cells).

The activity or level of a lineage marker protein can be detected and/or quantified by detecting or quantifying the expressed polypeptide. The polypeptide can be detected and quantified by any of a number of means well known to those of skill in the art. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binderlig and assays, immunohistochemical techniques, agglutination, complement assays, high performance liquid chromatography (HPLC), thin layer chromatography (TLC), hyperdiffusion chromatography, and the like.

Described below are non-limiting examples of techniques that may be used to detect marker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-marker protein antibodies (unlabeled) are then brought into contact with the support and assayed by a secondary immunological reagent, such as labeled protein A or anti-immunoglobulin (suitable labels including ¹²⁵I, horseradish peroxidase, alkaline phosphatase, fluorophore). Chromatographic detection may also be used. [0125] Immunohistochemistry may be used to detect expression of marker protein. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy. Anti-marker protein antibodies, such as intrabodies, can also be used for imaging purposes, for example, to detect the presence of marker protein in cells or, e g., an embryo. Suitable labels include radioisotopes, iodine (¹²⁵I, ¹²¹I), carbon (¹⁴C), sulphur (³⁵S), tritium (³H), indium (¹¹²In), and technetium (“mTc), fluorescent labels, such as fluorescein and rhodamine, and biotin. Antibodies that may be used to detect marker protein include any antibody, whether natural or synthetic, full length or a fragment thereof, monoclonal or polyclonal, that binds sufficiently strongly and specifically to the marker protein to be detected. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the marker protein relative to other proteins, such as related proteins.

Antibodies are commercially available or may be prepared according to methods known in the art. Antibodies and derivatives thereof that may be used encompass polyclonal or monoclonal antibodies, chimeric, human, humanized, primatized (CDR-grafted), veneered or single-chain antibodies as well as functional fragments, i.e., marker protein binding fragments, of antibodies. For example, antibody fragments capable of binding to a marker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain. In some embodiments, agents that specifically bind to a marker protein other than antibodies are used, such as peptides. Peptides that specifically bind to a marker protein can be identified by any means known in the art. For example, specific peptide binders of a marker protein can be screened for using peptide phage display libraries.

### Nucleic Acids

The methods described herein include providing engineered cells (e.g., ESCs or TSCs) that over-express at least one cadherin.

Providing the plurality of engineered ESCs can comprise: (i) providing an expression construct comprising a nucleic acid encoding E-cadherin, operably linked to at least one expression control element permitting gene expression in mammalian cells; and (ii) introducing the expression construct into ESCs in a manner permitting expression of the introduced construct in at least one of the ESCs, thereby generating at least one engineered ESC. Providing the plurality of engineered ESCs can comprise culturing the at least one engineered ESC of (ii).

Providing the plurality of engineered TSCs can comprise: (i) providing an expression construct comprising a nucleic acid encoding P-cadherin, operably linked to at least one expression control element permitting gene expression in mammalian cells; and (ii) introducing the expression construct into TSCs in a manner permitting expression of the introduced construct in at least one of the TSCs, thereby generating at least one engineered TSC. Providing the plurality of engineered TSCs can comprise culturing the at least one engineered TSC of (ii).

The expression control element can comprise a promoter, an enhancer, a 5′ un-translated region, a 3′ un-translated region, or any combination thereof. The promoter can be a ubiquitous promoter. The promoter can be a constitutive or an inducible promoter.

In some embodiments, the at least a portion of the plurality of engineered ESCs over-express E-cadherin relative to wild-type ESCs. In some embodiments, the at least a portion of the plurality of engineered TSCs over-express P-cadherin relative to wild-type TSCs. The plurality of XEN cells can be wild-type XEN cells. In some embodiments, none of the plurality of XEN cells are engineered to over-express E-cadherin, P-cadherin, or K-cadherin.

In some embodiments, the engineered ESC or the plurality of engineered ESCs over-expresses E-cadherin (e.g., relative to wild-type) by at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values). In some embodiments, the engineered TSC or the plurality of engineered TSCs over-expresses P-cadherin (e.g., relative to wild-type) by at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values). In some embodiments, the over-expression is determined by any method known in the art, including, for example, quantitative PCR, fluorescence-activated cell sorting (FACS), or western blot. Any of the methods described above for detecting, e.g., lineage markers, may also be used.

“Genetic construct” or “construct” as used herein shall be given their ordinary meanings and can also refer to nucleic acids that comprise a nucleotide sequence which encodes a gene product (e.g., an RNA and/or a protein). The nucleic acid can comprise at least one regulatory element for expression (e.g., an expression control element). The nucleic acid can comprise a vector, such as a viral vector. In some embodiments, the vector can comprise an adenovirus vector, an adeno-associated virus vector, an Epstein-Barr virus vector, a Herpes virus vector, an attenuated HIV vector, a retroviral vector, a vaccinia virus vector, or any combination thereof. In some embodiments, the vector can comprise an RNA viral vector. In some embodiments, the vector can be derived from one or more negative-strand RNA viruses of the order Mononegavirales. In some embodiments, the vector can be a rabies viral vector. Many such vectors useful for transferring exogenous genes into mammalian cells are available. The vectors may be episomal, e.g. plasmids, virus-derived vectors such cytomegalovirus, adenovirus, etc., or may be integrated into the cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Retroviral vectors can be “defective”, i.e. unable to produce viral proteins required for productive infection. Replication of the vector can require growth in the packaging cell line. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

In addition to viral vectors, a variety of additional tools have been developed that can be used for the incorporation of exogenous genes into cells. One such method that can be used for incorporating polynucleotides encoding target genes into cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In certain cases, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Exemplary transposon systems include the piggybac transposon (described in detail in, e.g., WO 2010/085699) and the sleeping beauty transposon (described in detail in, e.g., US2005/0112764), the disclosures of each of which are incorporated herein by reference.

As used herein, the term “expression vector” or “construct” refers to a vector that directs expression of an RNA or polypeptide (e.g., E-cadherin) from nucleic acid sequences contained therein linked to transcriptional regulatory sequences on the vector. The sequences can be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Gene products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector. One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector. Other non-integrative viral vectors contemplated herein are single-strand negative-sense RNA viral vectors, such Sendai viral vector and rabies viral vector. Another example of a non-integrative vector is a minicircle vector. Minicircle vectors are circularized vectors in which the plasmid backbone has been released leaving only the eukaryotic promoter and cDNA(s) that are to be expressed. As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of nonessential viral genes. The vector and/or particle may be utilized for the purpose of transferring nucleic acids into cells either in vitro or in vivo. Numerous forms of viral vectors are known in the art.

In some embodiment, the vectors can include a regulatory sequence that allows, for example, the translation of multiple proteins from a single mRNA. Non-limiting examples of such regulatory sequences include internal ribosome entry site (IRES) and 2A self-processing sequence. In some embodiments, the 2A sequence is a 2A peptide site from foot-and-mouth disease virus (F2A sequence). In some embodiments, the F2A sequence has a standard furin cleavage site. In some embodiments, the vector can also comprise regulatory control elements known to one of skill in the art to influence the expression of the RNA and/or protein products encoded by the polynucleotide within desired cells of the subject. In some embodiments, functionally, expression of the polynucleotide is at least in part controllable by the operably linked regulatory elements such that the element(s) modulates transcription of the polynucleotide, transport, processing and stability of the RNA encoded by the polynucleotide and, as appropriate, translation of the transcript. A specific example of an expression control element is a promoter, which is usually located 5′ of the transcribed sequence. Another example of an expression control element is an enhancer, which can be located 5′ or 3′ of the transcribed sequence, or within the transcribed sequence. Another example of a regulatory element is a recognition sequence for a microRNA. Another example of a regulatory element is an ration and the splice donor and splice acceptor sequences that regulate the splicing of said intron. Another example of a regulatory element is a transcription termination signal and/or a polyadenylation sequence.

Expression control elements and promoters include those active in a particular tissue or cell type, referred to herein as a “tissue-specific expression control elements/promoters.” Tissue-specific expression control elements are typically active in specific cell or tissue (for example in the liver, brain, central nervous system, spinal cord, eye, retina or lung). Expression control elements are typically active in these cells, tissues or organs because they are recognized by transcriptional activator proteins, or other regulators of transcription, that are unique to a specific cell, tissue or organ type.

Expression control elements also include ubiquitous or promiscuous promoters/enhancers which are capable of driving expression of a polynucleotide in many different cell types. Such elements include, but are not limited, to the cytomegalovirus (CMV) immediate early promoter/enhancer sequences, the Rous sarcoma virus (RSV) promoter/enhancer sequences and the other viral promoters/enhancers active in a variety of mammalian cell types; promoter/enhancer sequences from ubiquitously or promiscuously expressed mammalian genes including, but not limited to, beta actin, ubiquitin or EF1 alpha; or synthetic elements that are not present in nature.

Expression control elements also can confer expression in a manner that is regulatable, that is, a signal or stimuli increases or decreases expression of the operably linked polynucleotide. A regulatable element that increases expression of the operably linked polynucleotide m response to a signal or stimuli is also referred to as an “inducible element” (that is, it is induced by a signal). Particular examples include, but are not limited to, a hormone (for example, steroid) inducible promoter. A regulatable element that decreases expression of the operably linked polynucleotide in response to a signal or stimuli is referred to as a “repressible element” (that is, the signal decreases expression such that when the signal, is removed or absent, expression is increased). Typically, the amount of increase or decrease conferred by such elements is proportional to the amount of signal or stimuli present: the greater the amount of signal or stimuli, the greater the increase or decrease in expression.

A promoter may promote ubiquitous expression or tissue-specific expression of an operably linked nucleic acid (e.g., engineered nucleic acid) sequence from any species, including humans. In some embodiments, the promoter is a eukaryotic promoter. Non-limiting examples of eukaryotic promoters include TDH3, PGK1, PKC1, TDH2, PYK1, TPI1, AT1, CMV, EF1 alpha, SV40, PGK1 (human or mouse), Ubc, human beta actin, CAG, TRE, UAS, Ac5, Polyhedrin, CaMKIIa, GAL1, GAL10, TEF1, GDS, ADH1, CaMV35S, Ubi, H1, and U6, as would be known to one of ordinary skill in the art (see, e.g., Addgene website: blog.addgene.org/plasmids-101-the-promoter -region).

Non-limiting examples of ubiquitous promoters include tetracycline-responsive promoters (under the relevant conditions), CMV (EF1 alpha, a SV40 promoter, PGK1, Ubc, CAG, human beta actin gene promoter, a RSV promoter, an EFS promoter, and a promoter comprising an upstream activating sequence (UAS). In certain embodiments, the promoter is a mammalian promoter.

In some embodiments, a promoter of the present disclosure is suitable for use in AAV vectors. See, e.g., U.S. Patent Application Publication No. 2018/0155789, which is hereby incorporated by reference in its entirety for this purpose.

Non-limiting examples of constitutive promoters include CP1, CMV, EF1 alpha, SV40, PGK1, Ubc, human beta actin, beta tubulin, CAG, Ac5, Rosa26 promoter, COL1A1 promoter, polyhedrin, TEF1, GDS, CaM3 5S, Ubi, H1, U6, red opsin promoter (red promoter), rhodopsin promoter (rho promoter), cone arrestin promoter (car promoter), rhodopsin kinase promoter (rk promoter). In some instances, the constitutive promoter is a Rosa26 promoter. In some instances, the constitutive promoter is a COL1A1 promoter.

An “inducible promoter” is one that is characterized by initiating or enhancing transcriptional activity when in the presence of, influenced by, or contacted by an inducing agent. An inducing agent may be endogenous or a normally exogenous condition, compound, agent, or protein that contacts an engineered nucleic acid (e.g., engineered nucleic acid) in such a way as to be active in inducing transcriptional activity from the inducible promoter. In certain embodiments, an inducing agent is a tetracycline-sensitive protein (e.g., tTA or rtTA, TetR family regulators). Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (TetR, e.g., SEQ ID NO: 26, or TetRKRAB, e.g., SEQ ID NO: 27), a tetracycline operator sequence (tetO) and a tetracycline transactivator fusion protein (tTA), and a tetracycline operator sequence (tetO) and a reverse tetracycline transactivator fusion protein (rtTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), pH-regulated promoters, and light-regulated promoters. A non-limiting example of an inducible system that uses a light-regulated promoter is provided in Wang et al., Nat. Methods. 2012 Feb. 12; 9(3):266-9. Additional non-limiting examples of inducible promoters include mifepristone-responsive promoters (e.g., GAL4-E1b promoter) and coumermycin-responsive promoters. See, e.g., Zhao et al., Hum Gene Ther. 2003 Nov. 20; 14(17):1619-29.

A “reverse tetracycline transactivator” (“rtTA”), as used herein, is an inducing agent that binds to a TRE promoter (e.g., a TRE3G, a TRE2 promoter, or a P tight promoter) in the presence of tetracycline (e.g., doxycycline) and is capable of driving expression of a transgene that is operably linked to the TRE promoter. rtTAs generally comprise a mutant tetracycline repressor DNA binding protein (TetR) and a transactivation domain (see, e.g., Gossen et al., Science. 1995 Jun. 23; 268(5218):1766-9 and any of the transactivation domains listed herein). The mutant TetR domain is capable of binding to a TRE promoter when bound to tetracycline. See, e.g., U.S. Provisional Application No. 62/738,894, entitled MUTANT REVERSE TETRACYCLINE TRANSACTIVATORS FOR EXPRESSION OF GENES, which was filed on Sep. 28, 2018, under attorney docket number H0824.70300US00, and is herein incorporated by reference in its entirety.

Physical methods for introducing a polynucleotide into a cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A preferred method for the introduction of a polynucleotide into a cell is calcium phosphate transfection.

Chemical means for introducing a polynucleotide into a cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a cell. In some embodiments, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution.

Nucleic acids described herein can be introduced into cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

In some aspects, non-viral methods can be used to deliver a nucleic acid described herein into a cell. In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition. Exemplary methods of nucleic acid delivery using a transposon include a Sleeping Beauty transposon system (SBTS) and a piggyBac (PB) transposon system. In some embodiments, an engineered described herein are generated by using a combination of gene insertion using the SBTS and genetic editing using a nuclease (e.g., Zinc finger nucleases (ZFNs), Transcription Activator-Like Effector Nucleases (TALENs), the CRISPR/Cas system, or engineered meganuclease re-engineered homing endonucleases).

### Culture

In some embodiments of the methods disclosed herein, the method comprises: providing a plurality of engineered embryonic stem cells (ESCs), wherein at least a portion of the plurality of engineered ESCs over-express E-cadherin (Cdh1); providing a plurality of engineered trophoblast stem cells (TSCs), wherein at least a portion of the plurality of engineered TSCs over-express P-cadherin (Cdh3); and providing a plurality of extra-embryonic (XEN) cells

The plurality of engineered ESCs can comprise at least 5000 ESCs. The plurality of engineered ESCs can comprise 6000-7000 (e.g. about 6000, 6100, 6200, 6300, 6400, 6500, 6600, 6700, 6800, 6900, 7000 or a number or a range between any two of these values) ESCs. The plurality of engineered TSCs can comprise at least 10000 TSCs. The plurality of engineered TSCs can comprise 15000-19000 (e.g. about 15000, 15500, 16000, 16500, 17000, 17500, 18000, 18500, 19000 or a number or a range between any two of these values) TSCs. The plurality of XEN cells can comprise at least 5000 XEN cells. The plurality of XEN cells can comprise 5000-6000 (e.g., about 5000, 5100, 5200, 5300, 5400, 5500, 5600, 5700, 5800, 5900, 6000 or a number or a range between any two of these values) XEN cells. The ESCs, the TSCs, and/or the XEN cells can be derived from a mammalian natural embryo. The mammalian natural embryo can be a mouse or human natural embryo.

The co-culturing can be performed using a physical substrate (e.g., a culture dish or plate). In some embodiments, the culture plate comprises one or more wells or microwells. The co-culturing can be performed in an inverted pyramidal microwell. The inverted-pyramidal microwell can be about 400 μm or about 800 μm in size (or e.g., about 400 μm, 500 μm, 600 μm, 700 μm, 800 μm or more). The inverted-pyramidal microwell can be about 400 μm or about 800 μm diameter (or e.g., about 400 μm, 500 μm, 600 μm, 700 μm, 800 μm or more).

The methods disclosed herein can be applied in culture volumes of any appropriate size. For example, the culture volume per embryo may be about 50 μl to about 10 ml, optionally about 100 μl to about 5 ml, optionally about 250 μl to about 5 ml, optionally about 1 ml to about 5 ml. The culture volume per embryo may be about 100, 150, 200, 250, 300, 350, 400, 450, 500, 550, 600, 650, 700, 750, 800, 850, 900, 950, 1000, 1500, 2000 μl, or more.

### Media

Provided herein are culture media (e.g., a first, second, and third culture media) for generating the synthetic embryos of the disclosure. In some embodiments, the method comprises contacting the plurality of engineered ESCs, the plurality of engineered TSCs and the plurality of extra-embryonic (XEN) cells with a first culture media to form a co-culture. The method can comprise replacing the first culture media with a second culture media about three days after the contacting step. The first culture media and the second culture media each can comprise a basal culture medium. The basal medium may comprise water, salts, amino acids, a carbon source, vitamins, lipids and a buffer. Suitable carbon sources may be assessed by one of skill in the art from compounds such as glucose, sucrose, sorbitol, galactose, mannose, fructose, mannitol, maltodextrin, trehalose dihydrate, and cyclodextrin. Basal media are commercially available, for example, under the trade names Advanced DMEM/F12 (Gibco, 12634-010) and CMRL-1066 (Invitrogen or Sigma). The basal culture medium can comprise Dulbecco's Modified Eagle Medium (DMEM), DMEM Nutrient Mixture 12 (DMEM/F12), Roswell Park Memorial Institute (RPMI) medium 1640, Neurobasal®, Neurobasal® A, Connaught Medical Research Laboratory 1066 (CMRL-1066), or any combination thereof. The basal culture medium can comprise Dulbecco's Modified Eagle Medium (DMEM), DMEM Nutrient Mixture 12 (DMEM/F12), Roswell Park Memorial Institute (RPMI) medium 1640, Neurobasal®, Neurobasal® A, Connaught Medical Research Laboratory 1066 (CMRL-1066), or any combination thereof.

The first culture media and the second culture media each can comprise a non-human serum or serum substitute thereof, a reducing agent, and an antibiotic. The non-human serum or serum substitute can comprise fetal bovine serum, bovine serum albumin, KnockOut™ Serum Replacement, or any combination thereof. The reducing agent can comprise beta-mercaptoethanol (2-ME), N-acetyl-L-cysteine, dithiothreitol (DTT), or any combination thereof. The antibiotic can comprise Penicillin-streptomycin, Amphotericin B, Ampicillin, Erythromycin, Gentamycin, Kanamycin, Neomycin, Nystatin, Polymyxin B, Tetracycline, Thiabendazole, Tylosin, or any combination thereof. The first culture media and the second culture media each can comprise N2 supplement, B27 supplement, Insulin-Transferrin-Selenium-Ethanolamine (ITS-X), GlutaMax™, non-essential amino acids, ascorbic acid, sodium pyruvate or any combination thereof.

Each component of the culture medium described herein may be present in an amount such that the culture medium is suitable for supporting the self-assembly of stem cells into a synthetic embryo structure. The stem cells described herein, e.g., the plurality of engineered ES cells, can be individually cultured prior to the coculturing described herein in a suitable culture media described, for example, in U.S. Publication No. 2022/0308041 and PCT publication 2023/114754, the contents of which are incorporated herein by reference in their entireties.

In some embodiments, the amount of one or more of the components in a solution or media of the disclosure can comprise about 0.01% to about 40% (e.g., about 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, or a number or a range between any two of these values) volume per volume (% v/v), weight per volume (% w/v) or weight per weight (% w/w) of the medium.

Sodium pyruvate may be included in the culture medium at a concentration of about 0.05 mM to about 20 mM, about 0.1 mM to about 10 mM, about 0.25 mM about 5 mM, or about 0.5 mM to about 2.5 mM e.g., about 1 mM.

A culture media of the disclosure can comprise an amino acid selected from the group comprising L-glycine, L-alanine, L-asparagine, L-aspartic acid, L-glutamic acid, L-proline and L-serine. Non-essential amino acids may be included in the culture medium, for example, comprising glycine (about 1 mg/ml to about 25 mg/ml or about 5 mg/ml to about 10 mg/ml e.g., about 7.5 mg/ml), L-alanine (about 1 mg/ml to about 25 mg/ml or about 5 mg/ml to about 10 mg/ml e.g., about 9 mg/ml), L-asparagine (about 5 mg/ml to about 30 mg/ml or about 10 mg/ml to about 15 mg/ml e.g., about 13.2 mg/ml), L-aspartic acid (about 5 mg/ml to about 30 mg/ml or about 10 mg/ml to about 15 mg/ml e.g., about 13 mg/ml), L-glutamic acid (about 5 mg/ml to about 50 mg/ml or about 10 mg/ml to about 20 mg/ml e.g., about 15 mg/ml), L-proline (about 5 mg/ml to about 30 mg/ml or about 10 mg/ml to about 15 mg/ml e.g., about 11 mg/ml) and/or L-serine (about 5 mg/ml to about 30 mg/ml or about 10 mg/ml to about 15 mg/ml e.g., about 11 mg/ml). In some embodiments, culture medium may comprise L-glycine at a concentration of about 7.5 mg/ml, L-alanine at a concentration of about 9 mg/ml, L- asparagine at a concentration of about 13 mg/ml, L-aspartic acid at a concentration of about 13 mg/ml, L-glutamic acid at a concentration of about 14.5 mg/ml, L-proline at a concentration of about 11.5 mg/ml and L-serine at a concentration of about 10.5 mg/ml. Penicillin may be included in the culture medium at a concentration of about 1 unit/ml to about 500 units/ml, about 2 units/ml to about 250 units/ml, about 5 units/ml to about 100 units/ml, about 10 units/ml to about 50 units/ml, or about 20 units/ml to about 30 units/ml e.g., about 25 units/ml. Streptomycin may be included in the culture medium at a concentration of about 1 μg/ml to about 500 μg/ml, about 2 μg/ml to about 250 μg/ml, about 5 μg/ml to about 100 μg/ml, about 10 μg/ml to about 50 μg/ml, 25 or about 20 μg/ml to about 30 μg/ml e.g., about 25 μg/ml. The culture medium can comprise penicillin at a concentration of about 25 units/ml and/or streptomycin at a concentration of about 25 μg/ml.

The culture medium may be free of serum or substantially free of serum or essentially free of serum. The culture medium may comprise a serum replacement medium. Such serum replacement media are commercially available under the trade names KSR (KnockOut™ Serum Replacement, Invitrogen, 10828-010) and N2B27 (e.g., Invitrogen, ME100137L1). The serum replacement medium may be included in the culture medium at about 5% to about 60%, about 10% to about 50%, about 15% to about 45%, or about 20% to about 40%, e.g., about 30%. In some embodiments, the in vitro culture medium is free of serum or substantially free of serum and comprises 30% serum replacement. The concentration or amount of one or more of the components in a solution or media can vary. For example, the concentration or amount of non-human serum or serum substitute thereof, a reducing agent, and/or an antibiotic can be adjusted as needed by one of skill in the art.

In some embodiments, the amount of non-human serum or serum substitute thereof can comprise about 0.01% to about 40% (e.g., about 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, or a number or a range between any two of these values) volume per volume (% v/v), weight per volume (% w/v) or weight per weight (% w/w) of the medium. In some embodiments, the amount of antibiotic can comprise about 0.01% to about 10% (e.g., about 0.01%, 0.1%, 1%, 2%, 3%, 4%, 5%, 6%, 7%, 8%, 9%, 10% or a number or a range between any two of these values) volume per volume (% v/v), weight per volume (% w/v) or weight per weight (% w/w) of the medium. The amount of e.g., the reducing agent can vary. For example, in some embodiments, the concentration of the reducing agent in the composition can be about 0.1 μM to about 1 mM (e.g., about 0.1, 0.2, 0.3, 0.4, 0.5, 0.6, 0.7, 0.8, 0.9, 1, 10, 100, 200, 300, 400, 500, 600, 700, 800, 900 μM, 1 mM, or a number or a range between any two of these values). Where percentages are provided for agents, ingredients and compounds, they can be % w/w, % w/v or % v/v with respect to the formulation as a whole, unless otherwise indicated.

The first culture media can comprise DMEM, FBS, GlutaMax™, 2-ME, non-essential amino acids, sodium pyruvate, HEPES, and Penicillin-streptomycin. The first culture medium can comprise a ROCK inhibitor. The first culture media can comprise DMEM, 12.5% FBS, 2 mM GlutaMax™, 0.1 mM 2-ME, 0.1 mM non-essential amino acids, 1 mM sodium pyruvate, 0.02 M HEPES, 1% Penicillin-streptomycin, and 7.5 nM ROCK inhibitor. The first culture media can comprise DMEM, 12.5% FBS, 2 mM GlutaMax™, 0.1 mM 2-ME, 0.1 mM non-essential amino acids, 1 mM sodium pyruvate, 0.02 M HEPES, and 1% Penicillin-streptomycin.

The first culture media of step (d) can comprise a ROCK inhibitor. In some embodiments, the first culture media does not comprise ROCK inhibitor. The method can comprise removing the ROCK inhibitor following about 24 hr of co-culture in the first culture media. Rho-associated protein kinase (e.g., ROCK) inhibitors include, but are not limited to N-[(1S)-2-Hydroxy-1-phenylethyl]-N′-[4-(4-pyridinyl)phenyl]-urea (AS 1892802), fasudil hydrochloride (also known as HA 1077), -[3-[[2-(4-Amino-1,2,5-oxadiazol-3-yl)-I-ethyl-1H-imidazo[4,5-c]pyridin-6-yl]oxy]phenyl]-4-[2-(4-morpholinyl)ethoxy]benzamide (GSK269962), 4-[4-(Trifluoromethyl)phenyl]-N-(6-Fluoro-1H-indazol-5-yl)-2-methyl-6-oxo-1,4,5,6-tetrahydro-3-pyridinecarboxamide (GSK 429286), (S)-(+)-2-Methyl-1-[(4-methyl-5-isoquinolinyl)sulfonyl]-hexahydro-1H-1,4-diazepine dihydrochloride (H 1152 dihydrochloride), (S)-(+)-4-Glycyl-2-methyl-1-[(4-methyl-5-isoquinolinyl)sulfonyl]-hexahydro-1H-1,4-diazepine dihydrochloride (glycyl-H 1152 dihydrochloride), N-[(3-Hydroxyphenyl)methyl]-N′-[4-(4-pyridinyl)-2-thiazolyl]urea dihydrochloride (RKI 1447 dihydrochloride), (3S)-1-[[2-(4-Amino-1,2,5-oxadiazol-3-yl)-1-ethyl-1H-imidazo [4,5-c]pyridin-7-yl]carbonyl]-3-pyrrolidinamine dihydrochloride (SB 772077B dihydrochloride), N-[2-[2-(Dimethylamino)ethoxy]-4-(1H-pyrazol-4-yl)phenyl-2,3-dihydro-1,4-benzodioxin-2-carboxamide dihydrochloride (SR 3677 dihydrochloride), and trans-4-[®-1-Aminoethyl]-N-4-pyridinylcyclohexanecarboxamide dihydrochloride (Y-27632 dihydrochloride), N-Benzyl-[2-(pyrimidin-4-yl)amino]thiazole-4-carboxamide (Thiazovivin), Rock Inhibitor, a isoquinolinesulfonamide compound (Rho Kinase Inhibitor), N-(4-Pyridyl)-N′-(2,4,6-trichlorophenyl)urea (Rho Kinase Inhibitor II), 3-(4-Pyridyl)-1H-indole (Rho Kinase Inhibitor III, Rockout), and 4-pyrazoleboronic acid pinacol ester; a Rock antibody commercially available from Santa Cruz Biotechnology selected from the group consisting of Rock-1 (B 1), Rock-1 (C-19), Rock-1 (H-11), Rock-1 (G-6), Rock-1 (H-85), Rock-1 (K-18), Rock-2 (C-20), Rock-2 (D-2), Rock-2 (D-1 1), Rock-2 (N-19), Rock-2 (H-85), Rock-2 (30-J); a ROCK CRISPR/Cas9 knockout plasmid selected from the group consisting of Rock-1 CRISPR/Cas9 KO plasmid (h), Rock-2 CRISPR/Cas9 KO plasmid (h), Rock-1 CRISPR/Cas9 KO plasmid (m), Rock-2 CRISPR/Cas9 KO plasmid (m); a ROCK siRNA, shRNA plasmid and/or shRNA lentiviral particle gene silencer selected from the group consisting of Rock-1 siRNA (h): sc-29473, Rock-1 siRNA (m): sc-36432, Rock-1 siRNA ®: sc-72179, Rock-2 siRNA (h): sc-29474, Rock-2 siRNA (m): sc-36433, Rock-2 siRNA ®: sc-108088. In some embodiments, the ROCK inhibitor comprises Y-27632.

The first culture media can comprise an effective amount of ROCK inhibitor, such as, for example, at a concentration of about 0.1 nM to about 100 nM (e.g., about 0.1, 0.2, 0.3, 0.4, 0.5, 1, 1.5, 2, 2.5, 3, 3.5, 4, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mM or a number or a range between any two of these values). In some embodiments, the first culture media comprises a ROCK inhibitor at a concentration of about 7.5 nM. In some embodiments, the first culture media comprises about 1 nM to about 100 nM Y-27632 (e.g., about 1, 1.5, 2, 2.5, 3, 3.5, 4, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nM or a number or a range between any two of these values). In some embodiments, the first culture media comprises about 7.5 nM Y-27632.

In some embodiments, the second and/or third culture media each comprise one or more hormones or analogues thereof. The term “analogue” is used in this specification to refer to a biologically active analogue of any of the components of the culture medium. Such an analogue may be natural or synthetic.

The specific biologically active ligands and compounds used in the media defined herein, such as insulin, progesterone, activin etc. are used for illustrative purposes. However, one of skill in the art will readily recognize that analogues of such ligands and compounds may equally be used as alternatives, provided that they retain the relevant biological activity. One of skill in the art will be able to identify, in a routine manner, other biologically active compounds that are suitable for use as substitutes. For instance, these may be naturally occurring compounds or compounds which can be made by synthetic or semi-synthetic methods.

The term “analogue” as used herein can refer to a compound which may be structurally related to the relevant molecule. The term “agonist” as used herein can refer to a compound which might not be structurally related to the relevant molecule. For example, an agonist may activate the relevant receptor by altering the conformation of the receptor. Nevertheless, in both cases the terms are used in this specification to refer to compounds or molecules which can mimic, reproduce or otherwise generally substitute for the specific biological activity of the relevant molecule.

The amount of the one or more hormones present in the culture media can vary. For example, in some embodiments, the second and/or third culture media can comprise about 1 ng/ml to about 100 mg/ml (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900 ng/ml, 1 μg/ml, 10, 20, 30, 40, 50, 60, 70, 80, 90, 100 μg/ml, 1 mg/ml, 10, 20, 30, 40, 50, 60, 70, 80, 90, 100 mg/ml or a number or a range between any two of these values) of one or more hormones (e.g., progesterone) and/or one or more growth factors (e.g., insulin or an insulin-like growth factor). In some embodiments, the second and/or third culture media can comprise about 0.5 nM to about 1 mM (e.g., about 0.5, 1, 1.5, 2, 2.5, 3, 3.5, 4, 4.5 5 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900 nM, 0.5 mM, 1 mM, or a number or a range between any two of these values) of a hormone and/or insulin or an insulin-like growth factor.

The second culture media can comprise DMEM/F12, FBS, GlutaMax™, Penicillin-streptomycin, ITS-X, β-estradiol, progesterone, and N-acetyl-L-cysteine. The second culture media can comprise DMEM/F12, 20% FBS, 2 mM GlutaMax™, 1% Penicillin-streptomycin, 1×ITS-X, 8 nM β-estradiol, 200 ng/ml progesterone, and 25 mM N-acetyl-L-cysteine.

The method can comprise: replacing the second culture media with a third culture media about one day after replacing the first culture media with the second culture media. The third culture media can comprise DMEM/F12, KnockOut™ Serum Replacement, GlutaMax™, Penicillin-streptomycin, ITS-X, β-estradiol, progesterone, and N-acetyl-L-cysteine. The third culture media can comprise DMEM/F12, 30% KnockOut™ Serum Replacement, 2 mM GlutaMax™, 1% Penicillin-streptomycin, 1×ITS-X, 8 nM β-estradiol, 200 ng/ml progesterone, and 25 mM N-acetyl-L-cysteine.

### Applications of the Synthetic Embryos Provided Herein

As is understood by the skilled artisan, the synthetic embryos of the disclosure can be applied in multiple ways. Some exemplary applications are described further below.

Provided herein include differentiated cells obtainable by any of the methods disclosed herein. The method can comprise: removing one or more cells from the synthetic embryo; and culturing the one or more cells to produce differentiated cells. In some embodiments, the differentiated cells are selected from the group comprising exocrine secretory epithelial cells, hormone secreting cells, cells of the integumentary system, cells of the nervous system, metabolism and storage cells, barrier function cells, extracellular matrix cells, contractile cells, blood and immune system cells, germ cells, nurse cells and interstitial cells.

Differentiated cells may be produced the compositions and methods of the disclosure. Examples of differentiated cells include cells that are derived primarily from the endoderm, cells that are derived primarily from the ectoderm and cells that are derived primarily from the mesoderm and cells that are derived primarily from the germ line. Cells that are derived primarily from endoderm include exocrine secretory epithelial cells and hormone secretory cells. Exocrine secretory epithelial cells include salivary gland cell, von Ebner's gland cell in tongue, mammary gland cell, lacrimal gland cell, ceruminous gland cell in ear, eccrine sweat gland dark cell, eccrine sweat gland clear cell, apocrine sweat gland cell, gland of Moll cell in eyelid, sebaceous gland cell, Bowman's gland cell in nose, Brunner's gland cell in duodenum, seminal vesicle cell, prostate gland cell, bulbourethral gland cell, Bartholin's gland cell, gland of Littre cell, uterus endometrium cell, isolated goblet cell of respiratory and digestive tracts, stomach lining mucous cell, gastric gland zymogenic cell, gastric gland oxyntic cell, pancreatic acinar cell, paneth cell of small intestine, type II pneumocyte of lung and Clara cell of lung. Hormone secreting cells include anterior pituitary cells, intermediate pituitary cell, magnocellular neurosecretory cells, gut and respiratory tract cells, thyroid gland cells, parathyroid gland cells, adrenal gland cells, Leydig cell of testes, Theca interna cell of ovarian follicle, corpus luteum cell, juxtaglomerular cell, macula densa cell of kidney, peripolar cell of kidney and Mesangial cell of kidney.

Cells that are derived primarily from ectoderm include cells of the integumentary system and nervous system. Cells of the integumentary system include keratinizing epithelial cells (such as epidermal keratinocyte, epidermal basal cell, keratinocyte of fingernails and toenails, nail bed basal cell, medullary hair shaft cell, cortical hair shaft cell, cuticular hair shaft cell, cuticular hair root sheath cell, hair root sheath cell of Huxley's layer, hair root sheath cell of Henle's layer, external hair root sheath cell, hair matrix cell), wet stratified barrier epithelial cells (such as surface epithelial cell of stratified squamous epithelium of cornea, tongue, oral cavity, esophagus, anal canal, distal urethra and vagina, basal cell of epithelia of cornea, tongue, oral cavity, esophagus, anal canal, distal urethra and vagina and urinary epithelium cell). Cells of the nervous system include sensory transducer cells (such as auditory inner hair cell of organ of Corti, auditory outer hair cell of organ of Corti, basal cell of olfactory epithelium, cold-sensitive primary sensory neurons, heat-sensitive primary sensory neurons, Merkel cell of epidermis, olfactory receptor neuron, pain-sensitive primary sensory neurons, photoreceptor cells of retina in eye, proprioceptive primary sensory neurons, touch-sensitive primary sensory neurons, type I carotid body cell, type II carotid body cell, type I hair cell of vestibular system of ear, type II hair cell of vestibular system of ear and type I taste bud cell), autonomic neuron cells (such as cholinergic neural cell, adrenergic neural cell and peptidergic neural cell), sense organ and peripheral neuron supporting cells (such as inner pillar cell of organ of Corti, outer pillar cell of organ of Corti, inner phalangeal cell of organ of Corti, outer phalangeal cell of organ of Corti, border cell of organ of Corti, Hensen cell of organ of Corti, vestibular apparatus supporting cell, taste bud supporting cell, 5 olfactory epithelium supporting cell, Schwann cell, satellite glial cell and enteric glial cell), central nervous system neurons and glial cells (such as astrocyte, neuron cells, oligodendrocyte and spindle neuron) and lens cells (such as anterior lens epithelial cell and crystallin-containing lens fiber cell).

Cells that are derived primarily from mesoderm include metabolism and storage cells, barrier function cells, extracellular matrix cells, contractile cells, blood and immune system cells, germ cells, nurse cells and interstitial cells. Metabolism and storage cells include hepatocyte, adipocytes and liver lipocyte. Barrier function cells (lung, gut, exocrine glands and urogenital tract) include kidney cells (such as kidney parietal cell, kidney glomerulus podocyte, kidney proximal tubule brush border cell, loop of Henle thin segment cell, kidney distal tubule cell, kidney collecting duct cell, type I pneumocyte, pancreatic duct cell, nonstriated duct cell, duct cell, intestinal brush border cell, exocrine gland striated duct cell, gall bladder epithelial cell, ductulus efferens nonciliated cell, epididymal principal cell and epididymal basal cell). Extracellular matrix cells include ameloblast epithelial cell, planum semilunatum epithelial cell of vestibular system of ear, organ of Corti interdental epithelial cell, loose connective tissue fibroblasts, corneal fibroblasts, tendon fibroblasts, bone marrow reticular tissue fibroblasts, other nonepithelial fibroblasts, pericyte, nucleus pulposus cell of intervertebral disc, cementoblast/cementocyte, Odontoblast/odontocyte, hyaline cartilage chondrocyte, fibrocartilage chondrocyte, elastic cartilage chondrocyte, osteoblast/osteocyte, osteoprogenitor cell, hyalocyte of vitreous body of eye, stellate cell of perilymphatic space of ear, hepatic stellate cell and pancreatic stelle cell. Contractile cells include skeletal muscle cells including red skeletal muscle cell, white skeletal muscle cell, intermediate skeletal muscle cell, nuclear bag cell of muscle spindle and nuclear chain cell of muscle spindle, satellite cells, heart muscle cells including ordinary heart muscle cell, nodal heart muscle cell and Purkinje fiber cell, smooth muscle cell, myoepithelial cell of iris and myoepithelial cell of exocrine glands. Blood and immune system cells include erythrocyte, megakaryocyte, monocyte, connective tissue macrophage, Langerhans cell, osteoclast, dendritic cell, microglial cell, neutrophil granulocyte, eosinophil granulocyte, basophil granulocyte, hybridoma cell, mast cell, helper T cell, suppressor T cell, cytotoxic T cell, natural Killer T cell, B cell, natural killer cell, reticulocyte and committed progenitors for the blood and immune system. Germ cells include oogonium/Oocyte, spermatid, spermatocyte, spermatogonium cell and spermatozoon. Nurse cells include ovarian follicle cell, sertoli cell and thymus epithelial cell, and interstitial cells include interstitial kidney cells.

The disclosed compositions and methods may be used to produce differentiated cells from any suitable mammalian species, such as: primates, including humans, great apes (e.g., gorillas, chimpanzees, orangutans), old world monkeys, new world monkeys; rodents (e.g., mice, rats, guinea pigs, hamsters); cats; dogs; lagomorphs (including rabbits); cows; sheep; goats; horses; pigs; and any other livestock, agricultural, laboratory or domestic mammals. The presently disclosed compositions and methods may be used to produce differentiated cells from any non-human mammal, including but not limited to those described above.

There are provided, in some embodiments, differentiated cells obtainable by the in vitro methods described herein. There are provided synthetic embryos generated by the in vitro methods disclosed herein. Disclosed herein are compositions for generating a synthetic embryo.

The synthetic embryos disclosed herein can have a variety of applications including, e.g., investigating mechanisms of embryonic development and for use in treating a subject for a disease or disorder.

Disclosed herein include methods for determining the effect of a test agent on embryonic development. In some embodiments, the method comprises: a) providing a synthetic embryo generated by any method provided herein; b) contacting the synthetic embryo with a test agent; and c) determining the effect of the test agent on the synthetic embryo. In some embodiments, the determining can comprise comparing a phenotype or a genotype of the synthetic embryo in the presence of the test agent with the phenotype or genotype of the synthetic embryo in the absence of the test agent.

The method can comprise generating the synthetic embryo by any method disclosed herein. The method can comprise contacting the synthetic embryo with the test agent prior to step (d). The method can comprise determining the subsequent effect on formation of the synthetic embryo. In some embodiments, determining the effect on the formation of a multicellular aggregate structure, egg cylinder structure, and/or post-implantation structure.

The method may comprise recording a plurality of images of the synthetic embryos. The plurality of images may be recorded over a pre-determined period of time, thus illustrating the development from the e.g., multicellular aggregates to the synthetic embryos. The imaging apparatus may comprise microscopy apparatus, suitable recording apparatus, and optionally image processing apparatus.

Typically, fluorescent markers, such as fluorescent dyes or fluorescent marker proteins, are used in the imaging of embryonic development. Such markers may be added to the culture system. For example, fluorescent dyes may be added to visualize particular molecules or cellular structures. For example, DAPI may be used to stain DNA or MitoTracker (Invitrogen) may be used to stain the mitochondria. Additionally or alternatively, synthetic embryo may produce such fluorescent markers endogenously, e.g., it may contain one or more cells which express a fluorescent marker protein. Such cells may have been genetically modified in order to confer the ability to express such a marker protein. Thus, fluorescence imaging apparatus may be particularly suitable for the methods described. The imaging apparatus may thus comprise a fluorescence microscope, such as a confocal microscope, that can include but is not limited to wide field, scanning and spinning disc confocal, and light sheet microscope.

Confocal microscopes image a single point of a specimen at any given time but allow generation of two dimensional or three dimensional images by scanning different points in a specimen in a regular raster to provide image data which can be assembled into a two or three dimensional image. For example, scanning a specimen in a single plane enables generation of a two dimensional image of a slice through the specimen. A plurality or “stack” of such two dimensional images can be combined to yield a three dimensional image. Spinning disc confocal microscopy provides added advantages over confocal laser scanning microscopy. Additionally, light sheet microscopy can also provide good imaging of embryonic development.

Disclosed herein include methods of identifying a compound useful for treating a disease. In some embodiments, the method comprises contacting the synthetic embryo or a differentiated cell(s) obtainable by any of the methods provided herein with the compound.

Disclosed herein include methods for diagnosing or treating a disease or disorder in a subject. In some embodiments, the method comprises use of a synthetic embryo, or a differentiated cell(s) obtainable by the methods provided herein, or any combination thereof. Also provided are methods of elucidating the role of a gene in embryo development. In some embodiments, the method comprises obtaining an ESC, a TSC, and/or a XEN cell where the gene has been modified or knocked out and culturing the cell to obtain a plurality of cells for use in the method for generating a synthetic embryo disclosed herein.

### EXAMPLES

Some aspects of the embodiments discussed above are disclosed in further detail in the following examples, which are not in any way intended to limit the scope of the present disclosure.

### Example 1

### Stem Cell-Derived Synthetic Embryos Self-Assemble by Exploiting Cadherin Codes and Cortical Tension

Mammalian embryos sequentially differentiate into trophectoderm and an inner cell mass, the latter of which differentiates into primitive endoderm and epiblast. Trophoblast stem (TS), extraembryonic endoderm (XEN) and embryonic stem (ES) cells derived from these three lineages can self-assemble into synthetic embryos. There is a need for methods for furthering understanding of the mechanisms that control these events. Disclosed herein is a stem cell-specific cadherin code that drives synthetic embryogenesis. The XEN cell cadherin code enables XEN cell sorting into a layer below ES cells, recapitulating the sorting of epiblast and primitive endoderm before implantation. The TS cell cadherin code enables TS cell sorting above ES cells, resembling extraembryonic ectoderm clustering above epiblast following implantation. Whereas differential cadherin expression drives initial cell sorting, cortical tension consolidates tissue organization. By optimizing cadherin code expression in different stem cell lines, the frequency of correctly formed synthetic embryos was tripled. Thus, by exploiting cadherin codes from different stages of development, lineage-specific stem cells bypass the preimplantation structure to directly assemble a postimplantation embryo.

Cadherins and protocadherins regulate cell adhesion forces in many different systems. Cells expressing different types and levels of cadherins show differential cell-cell adhesion and sorting. Moreover, synthetic genetic programs, in which distinct cell-cell contacts specify differential cadherin expression, can induce self-organization into multidomain structures and sequential assembly.

To determine the role of cadherins in the self-assembly of synthetic (e.g., ETX embryos) single-cell RNA sequencing (scRNA-seq) data was analyzed to examine cadherin expression in the building blocks of ETX embryos: embryonic stem (ES), trophoblast stem (TS) and extra-embryonic endoderm (XEN) cell lines (FIG. 1A). It was found that E-cadherin (Cdh1) messenger RNA (mRNA) was equally abundant in ES and TS cells, whereas P-cadherin (Cdh3) was expressed only in TS cells, and K-cadherin (Cdh6) was expressed mainly in XEN cells (FIG. 1B-FIG. 1C). The differential expression of cadherins in ES, TS and XEN cells indicated a role in driving the self-assembly of ETX embryos.

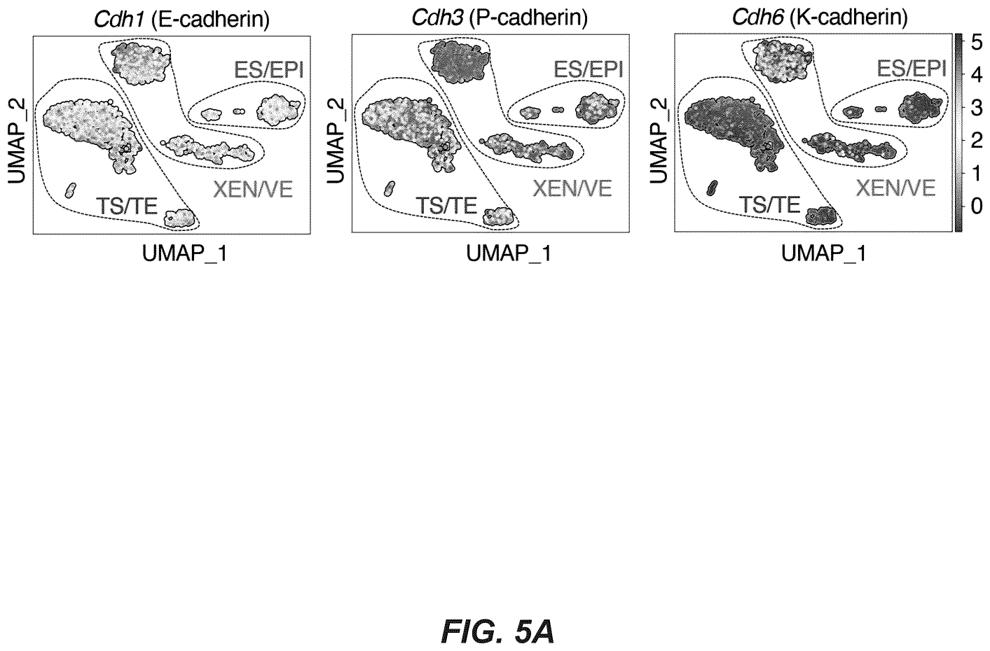

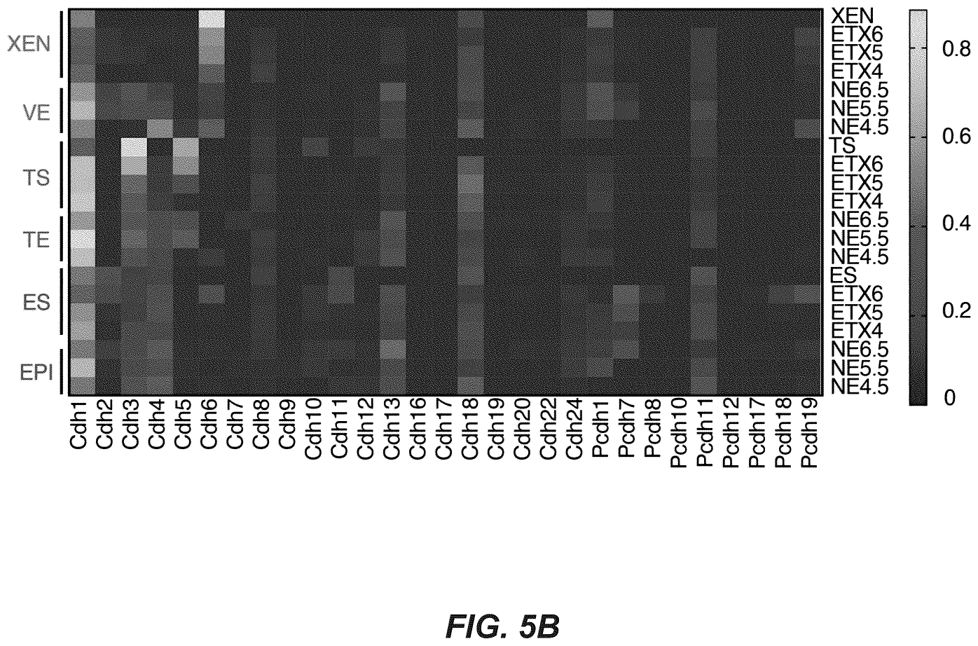

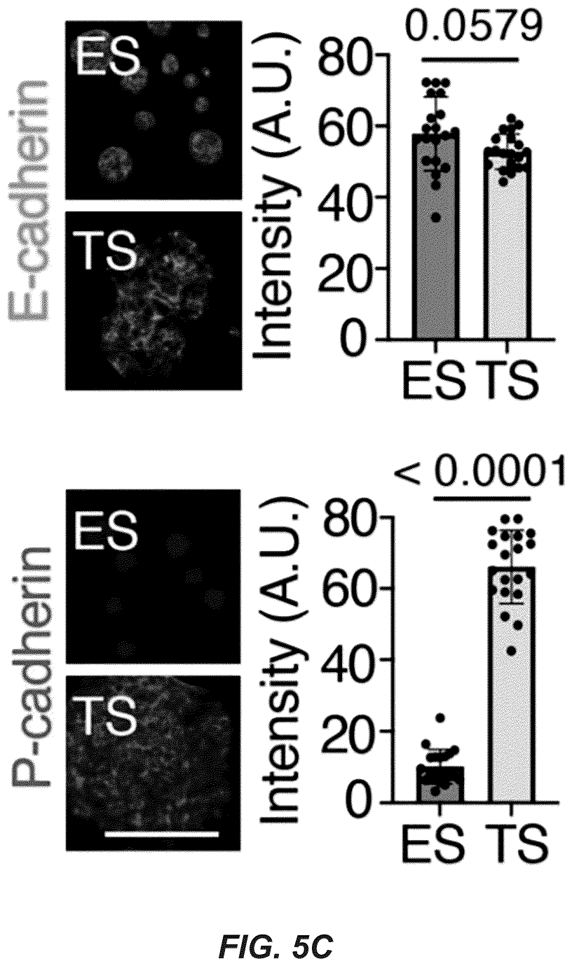

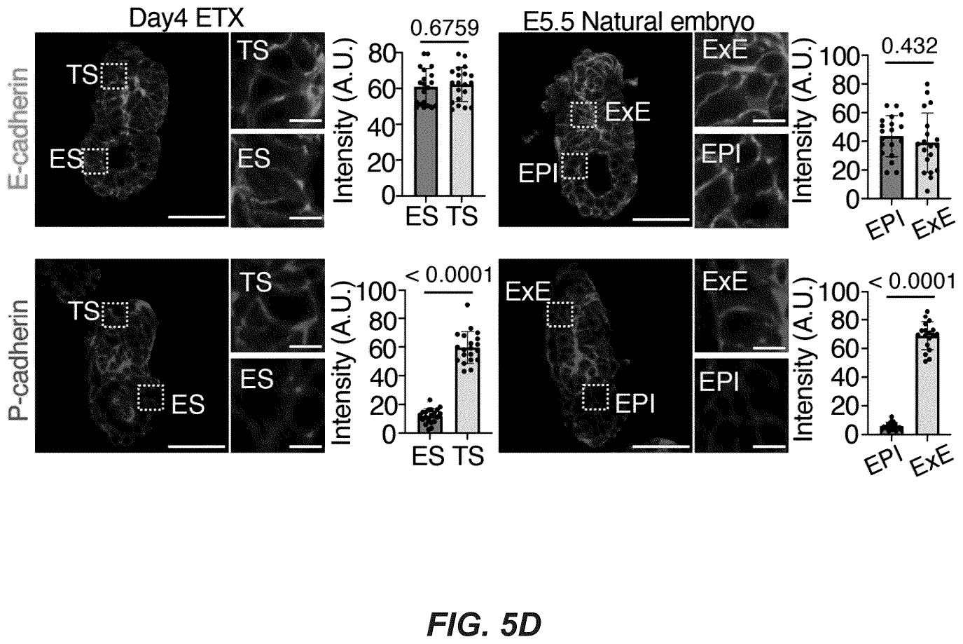

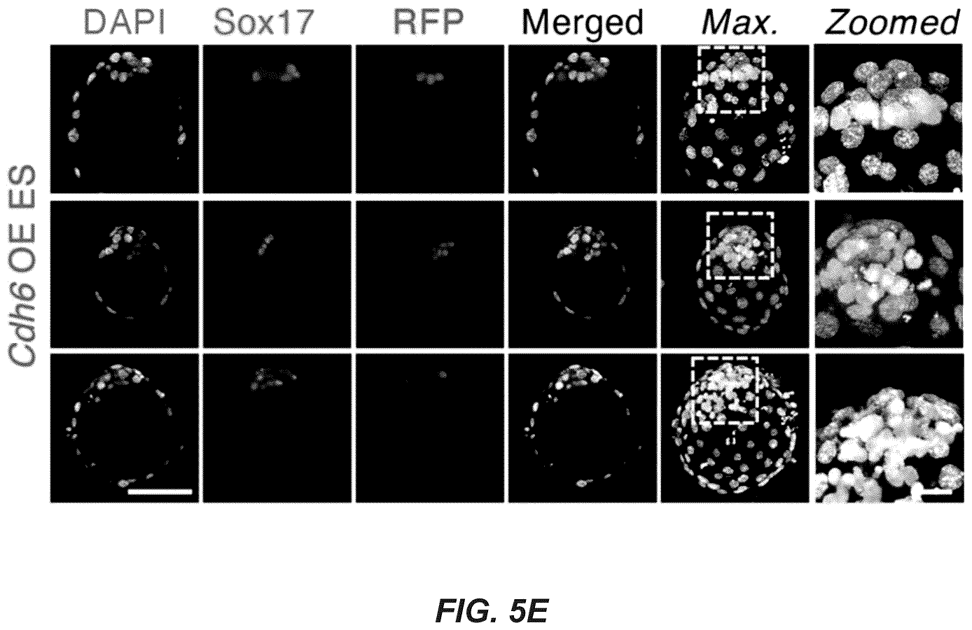

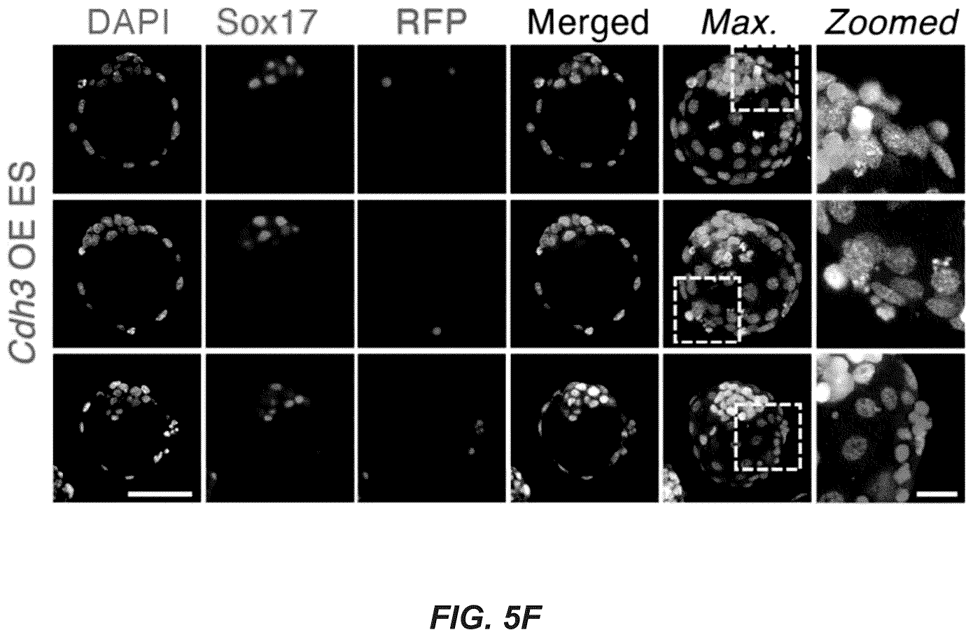

Next, the expression of these three cadherins in cells dissociated from either ETX or natural embryos at successive stages was examined (FIG. 1A). ES/epiblast, TS/trophectoderm and XEN/primitive endoderm lineages were defined by the expression of their respective markers and showed similar dynamics of cadherin expression in ETX and natural embryos (FIG. 1D and FIG. 5A). In natural embryos, E-cadherin was expressed in all lineages from E4.5 to E6.5; P-cadherin expression was elevated only in trophectoderm after implantation (E5.5 and E6.5); and K-cadherin expression was elevated only in primitive endoderm before implantation (E4.5) when it sorts below the epiblast FIG. 1D and FIG. 5B). The corresponding proteins, similar to mRNA, were verified to be differentially expressed in ES or TS colonies (FIG. 5C), day 4 ETX embryos and E5.5 natural embryos (FIG. 5D). Therefore, XEN cells most resemble E4.5 primitive endoderm cells of the preimplantation embryo, whereas TS cells resemble extraembryonic ectoderm cells of the postimplantation embryo.

ES cells readily form chimeras with eight-cell-stage embryos and sort to the epiblast lineage. Given that E- and K-cadherin were differentially expressed in the epiblast and primitive endoderm of natural preimplantation embryos, it was next examined whether overexpression (OE) of these cadherins in ES cells would affect their sub-sequent sorting in the blastocysts of chimeras (FIG. 1E). Wild-type ES cells (n=32 embryos) and ES cells overexpressing E-cadherin (Cdh1 OE) contributed exclusively to the epiblast of the chimeras (n=16 embryos) (FIG. 1F-FIG. 1G). In contrast, ES cells overexpressing K-cadherin (Cdh6 OE) frequently contributed to the primitive endoderm (n=16 embryos) (FIG. 1F-FIG. 1G and FIG. 5E). These data are consistent with K-cadherin promoting primitive endoderm localization and E-cadherin promoting epiblast localization.

ES cells overexpressing P-cadherin (Cdh3 OE) were excluded from the preimplantation embryo (n=13 embryos) and sorted outside the trophectoderm (FIG. 1F-FIG. 1G and FIG. 5F), consistent with low P-cadherin expression in all lineages of the blastocyst and elevated P-cadherin expression in the trophectoderm only in the postimplantation natural embryo.

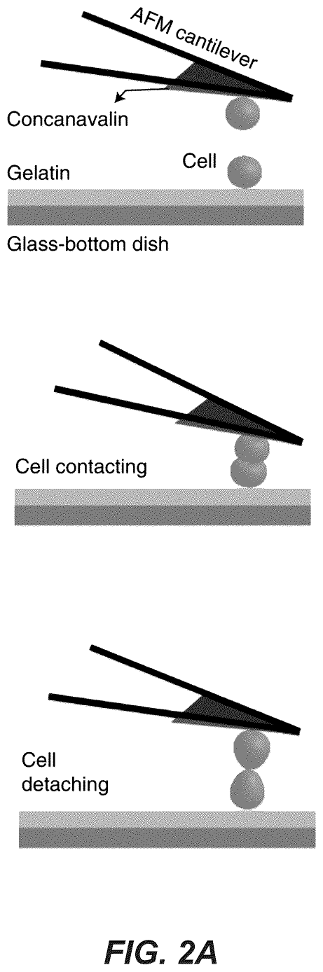

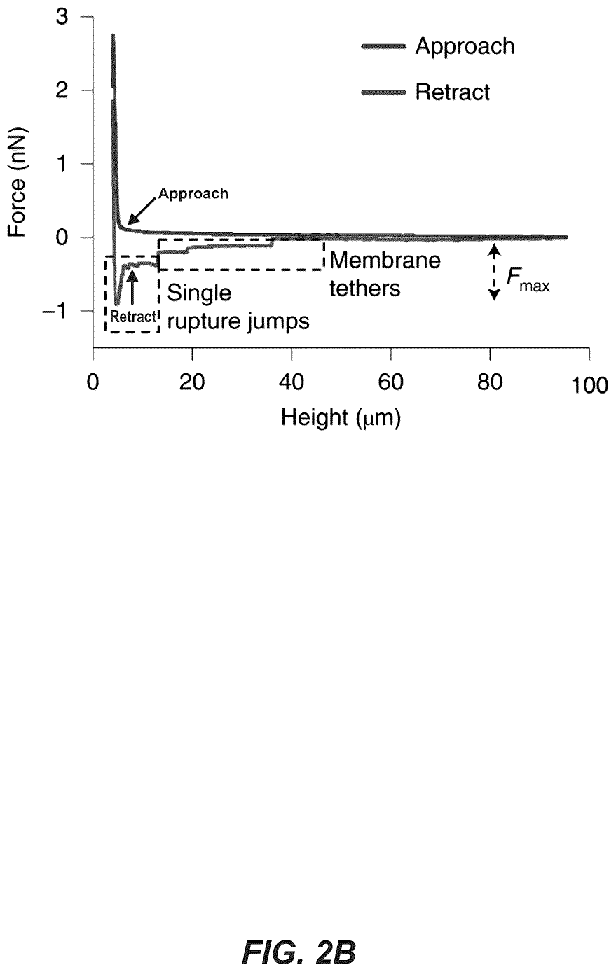

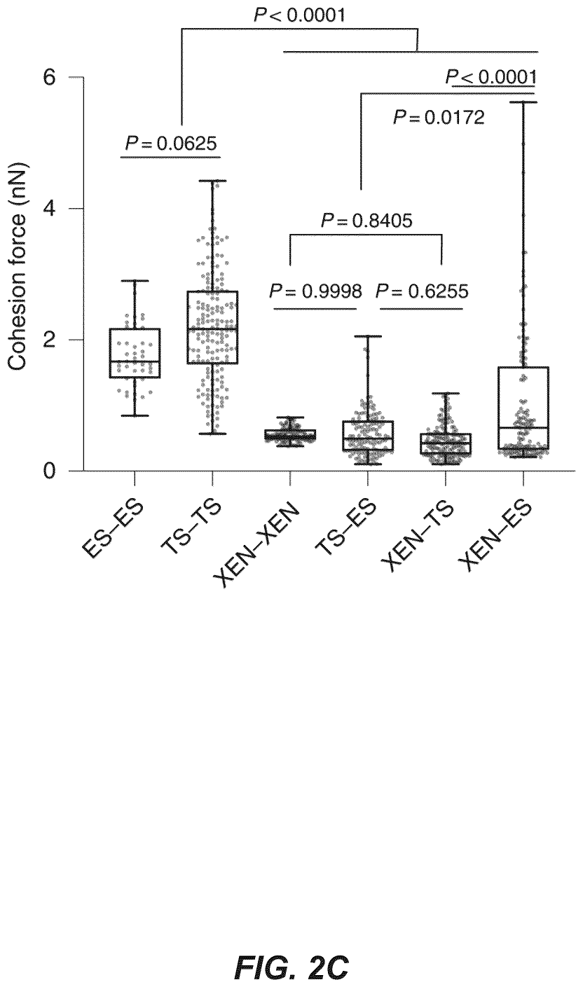

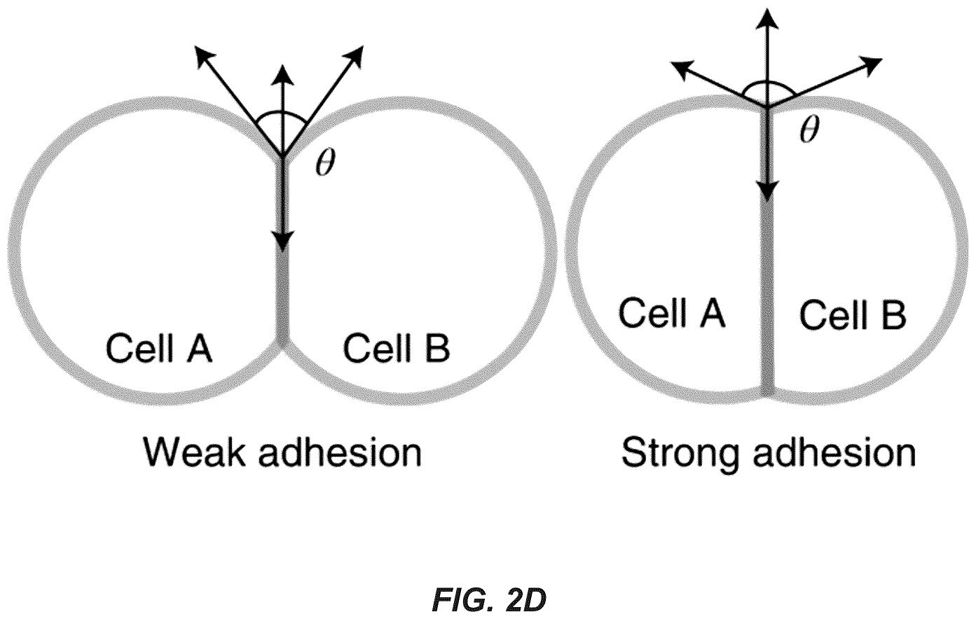

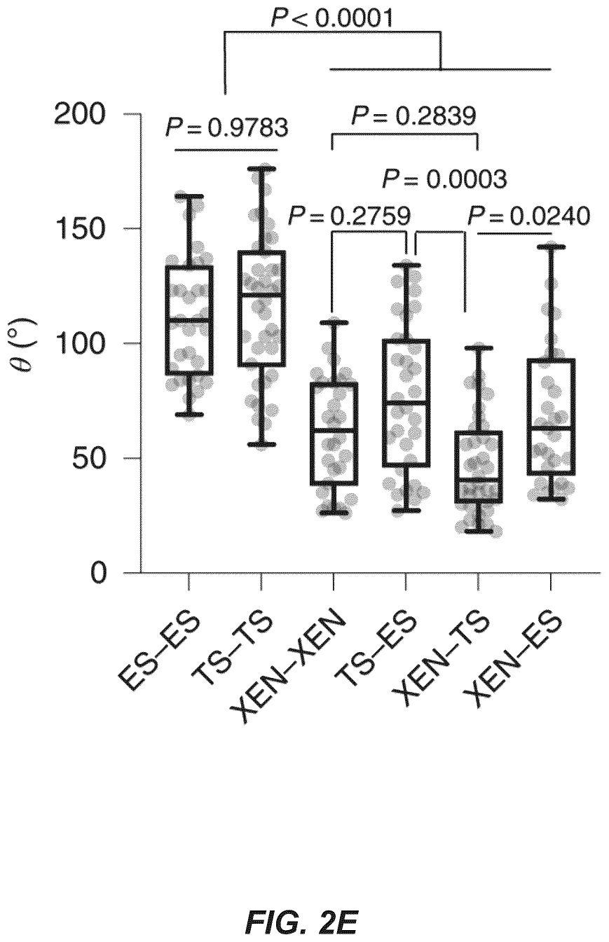

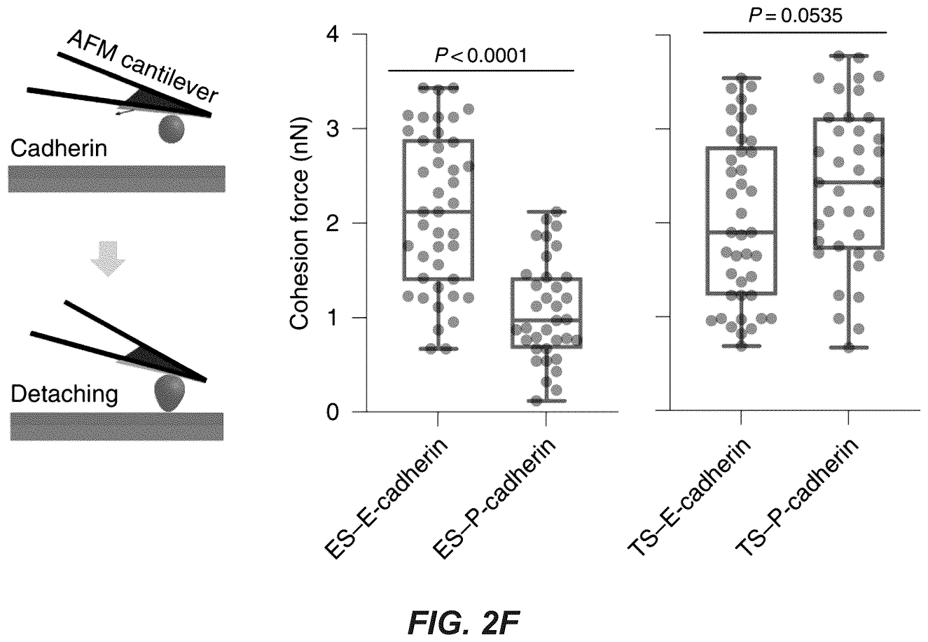

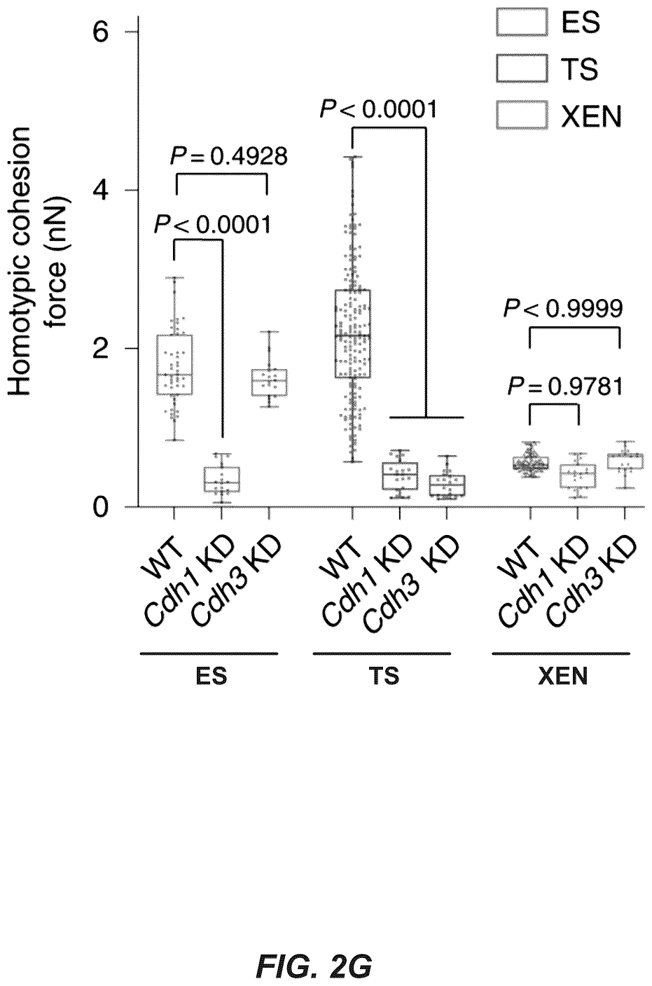

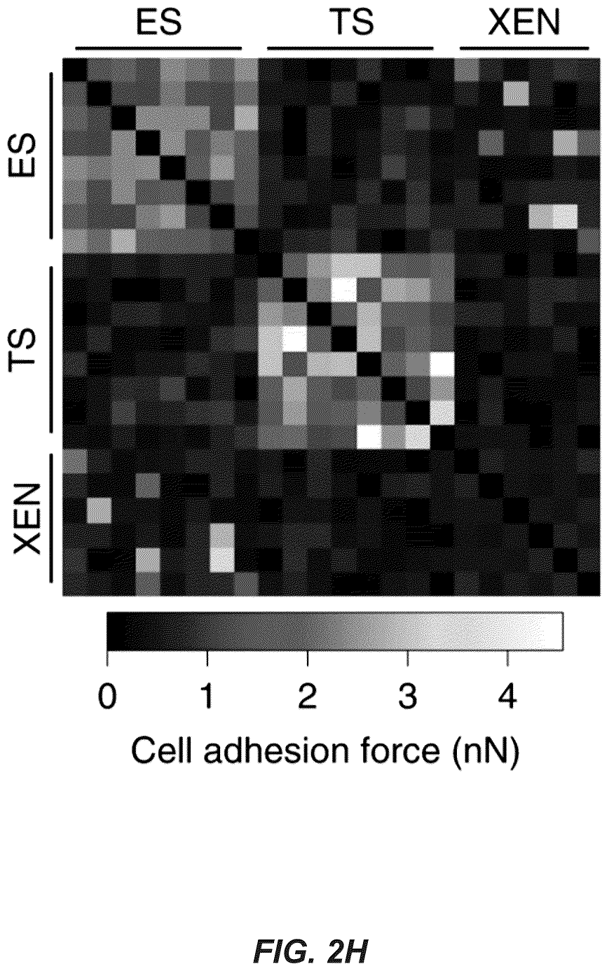

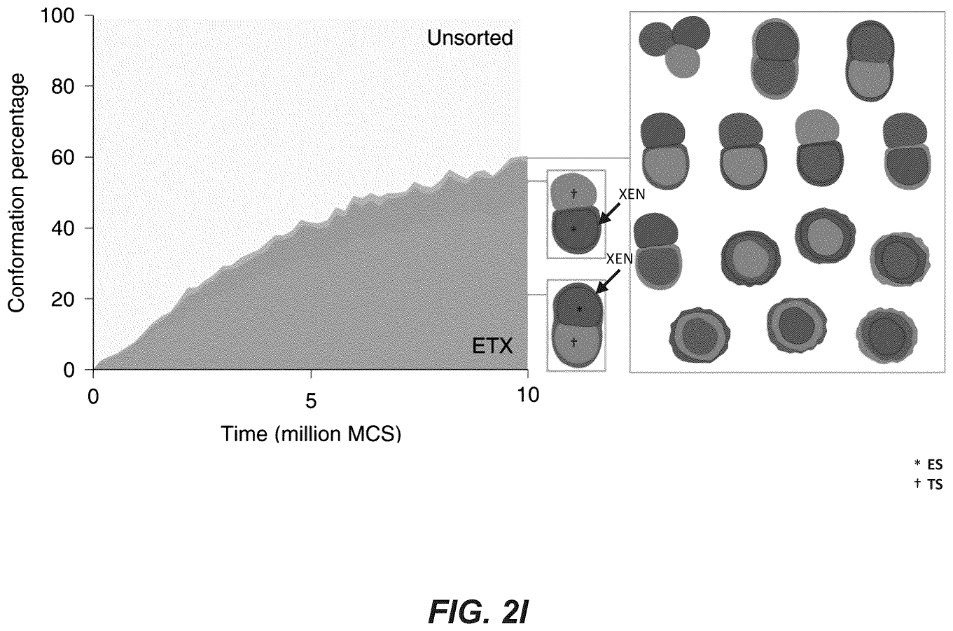

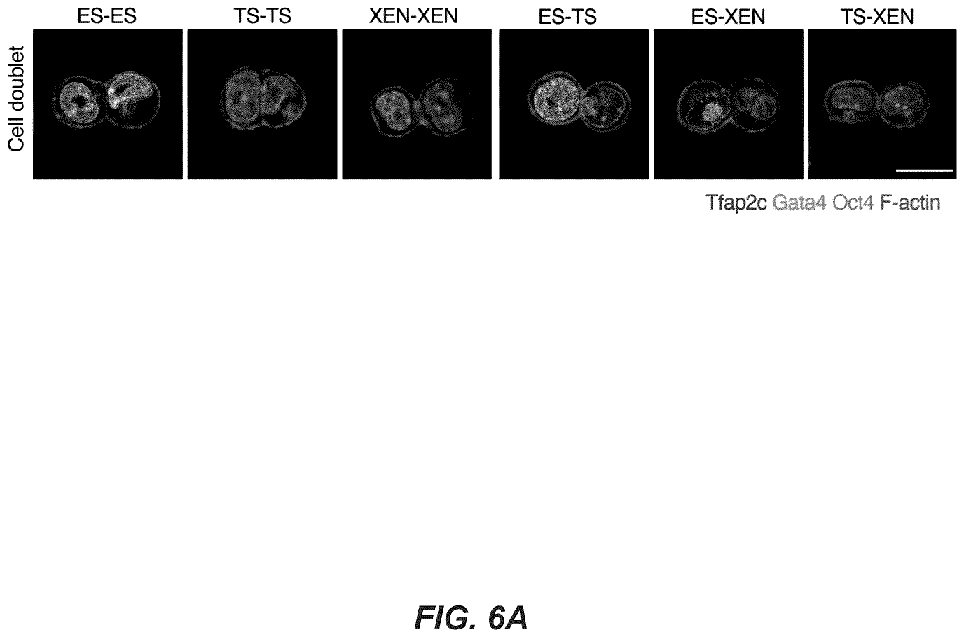

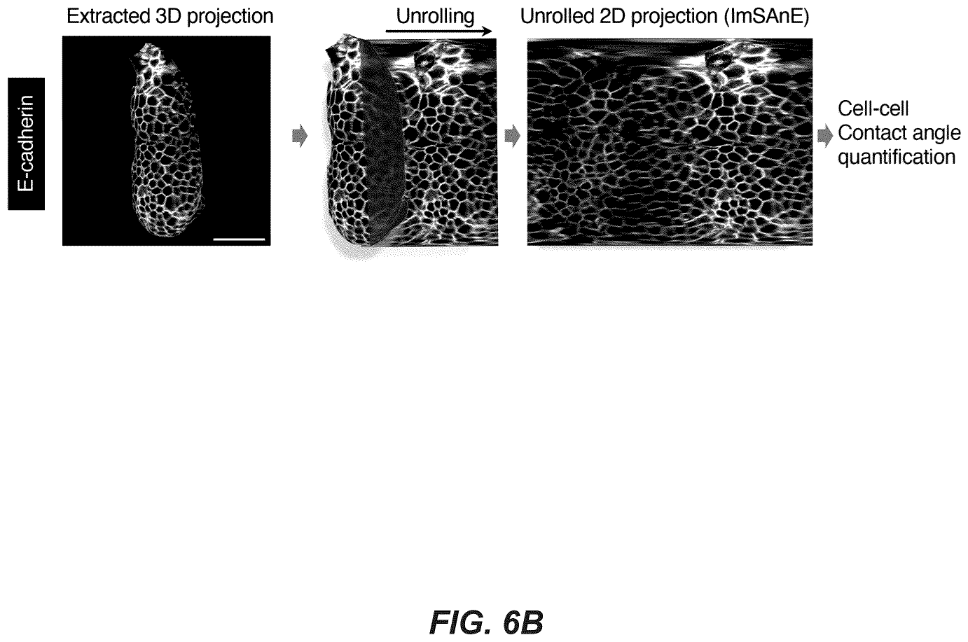

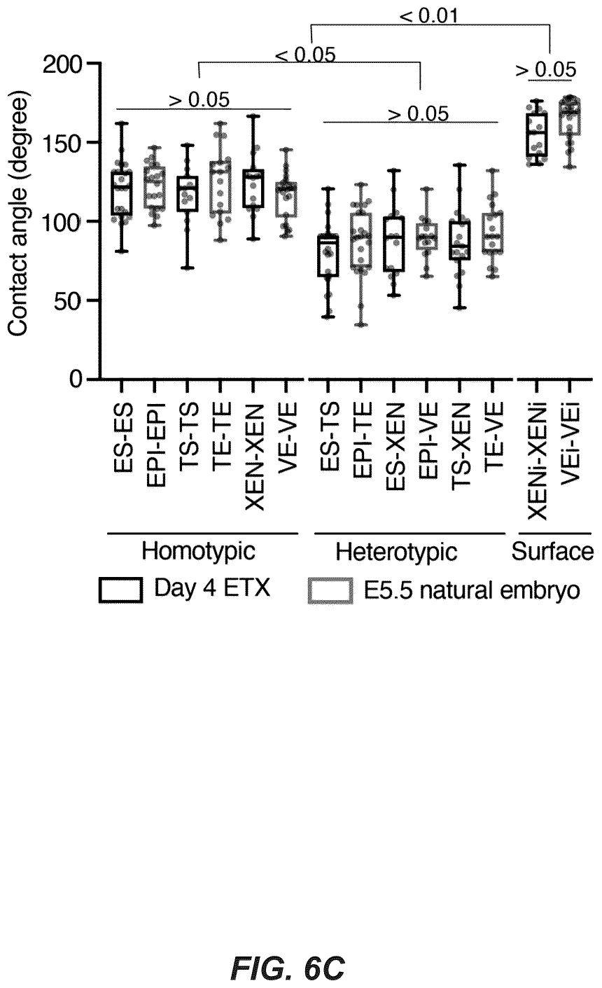

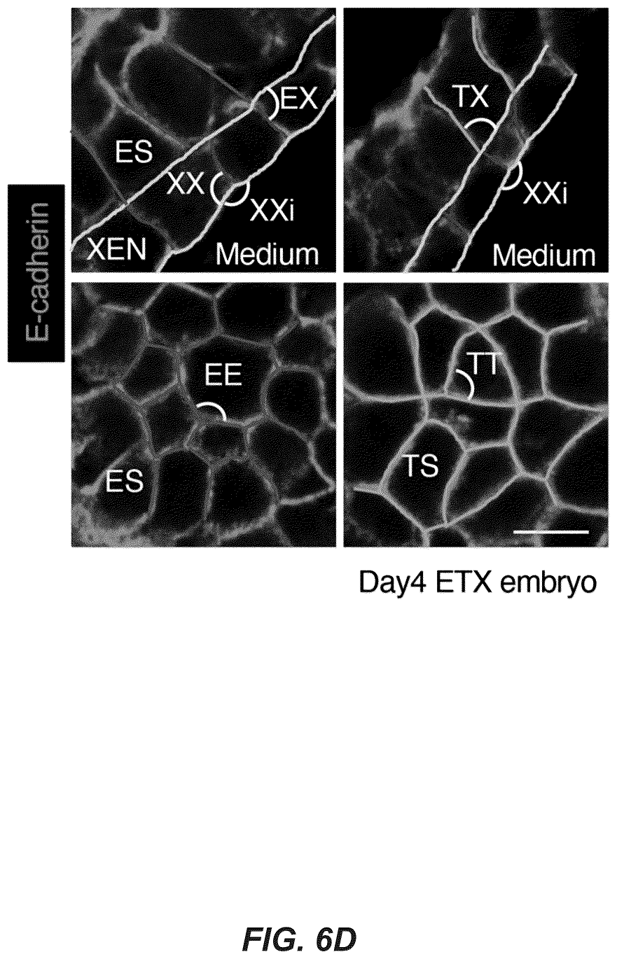

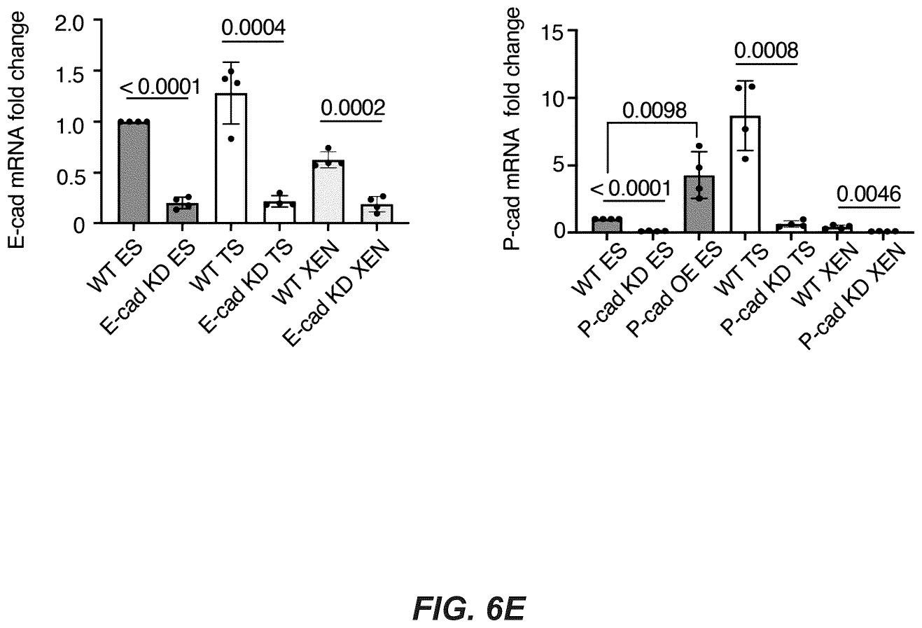

To evaluate whether differential adhesion plays a role in ETX embryo self-assembly, atomic force microscopy (AFM) was used to determine the cell-cell adhesion of ES, TS and XEN cells in vitro (FIG. 2A-FIG. 2B). The mean adhesion forces between ES-ES cell couples (1.94±0.54 nN) and TS-TS cell couples (2.20±0.85 nN) were significantly higher than those between XEN-XEN cell couples (0.55±0.11 nN) or between ES-TS cell couples (0.57±0.36 nN), thus indicating a tendency for ES cells and TS cells to form homotypic associations. In addition, the adhesion forces between XEN-ES cell couples (0.83±0.96 nN) were greater than those between XEN-XEN cell couples (0.55±0.11 nN) or XEN-TS (0.46±0.24 nN) cell couples, indicating that XEN cells have the highest affinity for ES cells (FIG. 2C). Adhesion forces from the contact angles between cells were calculated (FIG. 2D). The contact angles at ES-ES, TS-TS and XEN-ES junctions were greater than the contact angles between ES-TS, XEN-XEN and TS-XEN cells (FIG. 2E and FIG. 6A), in agreement with the AFM measurements.

To compare cell-cell contact angles in ETX and natural embryos, the imaging surface analysis environment (ImSAnE) algorithm was employed to extract focal planes from three-dimensional (3D) stacks of E-cadherin-stained day 4 ETX and E5.5 natural embryos and unrolled these into 2D projections (FIG. 6B). In agreement with cell contact angle measurements in stem cell doublets, the homotypic contact angles were larger than the heterotypic contact angles in ETX and natural embryos (FIG. 6C). It was also noted that the contact angle between XEN-XEN and VE-VE cells at the surface of ETX embryos and natural embryos was close to 180° (FIG. 6C-FIG. 6D), indicating a smooth boundary interface and reflecting the high relative tension along the interface after self-organization. Together, these measurements show differential cadherin expression and differential adhesion between stem cells that build, and lineages that comprise, ETX embryos.

To examine the potential relationship between differential cadherin expression and differential adhesion, adhesion forces between ES and TS cells and immobilized E-cadherin (Cdh1) or P-cadherin (Cdh3) substrate were measured (FIG. 2F). ES cells exhibited higher adhesion with immobilized E-cadherin (2.13±0.83 nN) than with P-cadherin (1.07±0.54 nN), whereas TS cells displayed comparable adhesion forces with both E-cadherin (2.02±0.89 nN) and P-cadherin (2.41±0.86 nN). RNA interference was next used to knockdown (KD) cadherins in stem cells. E-cadherin KD reduced ES-ES adhesion fourfold. P- or E-cadherin KD similarly reduced TS-TS adhesion, suggesting that downregulation of one cadherin was sufficient to decrease the TS-TS adhesion force below a critical threshold (FIG. 2G and FIG. 6E). Depletion of either E- or P-cadherin from XEN cells did not affect their homotypic adhesion (FIG. 2G). Thus, E-cadherin is required for the homotypic adhesion of ES cells, whereas both E- and P-cadherin are required for the homotypic adhesion of TS cells.

To assess whether the measured adhesion forces are sufficient to generate ETX embryos, assembly was simulated using the cellular Potts model (CPM), re-sampling AFM adhesion force measurements to provide parameters (FIG. 2H). This analysis showed that, among the many sorted configurations possible with three cell types, ETX-like structures were the most favored (FIG. 2I) (See, Modeling below).

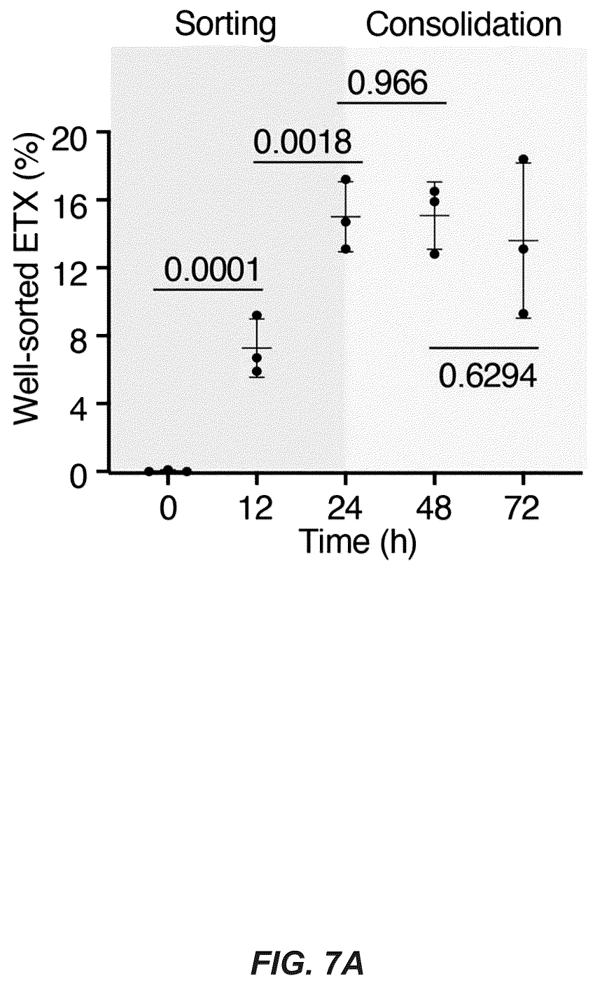

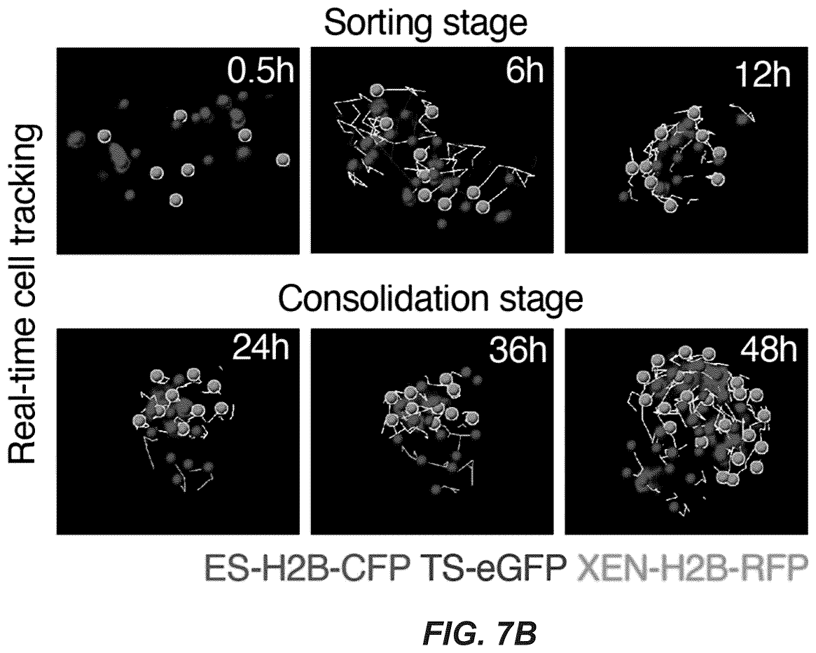

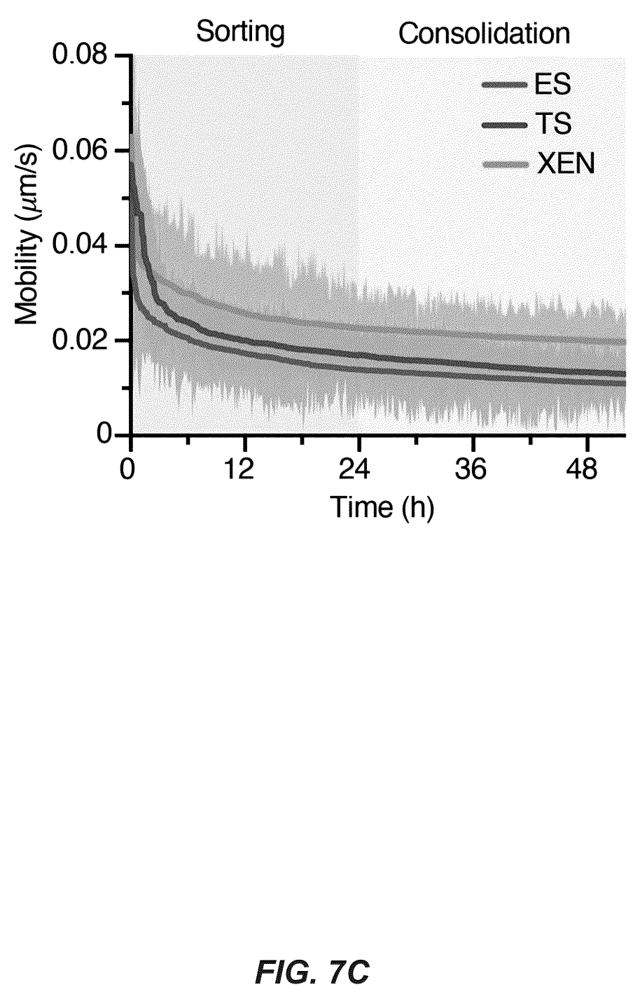

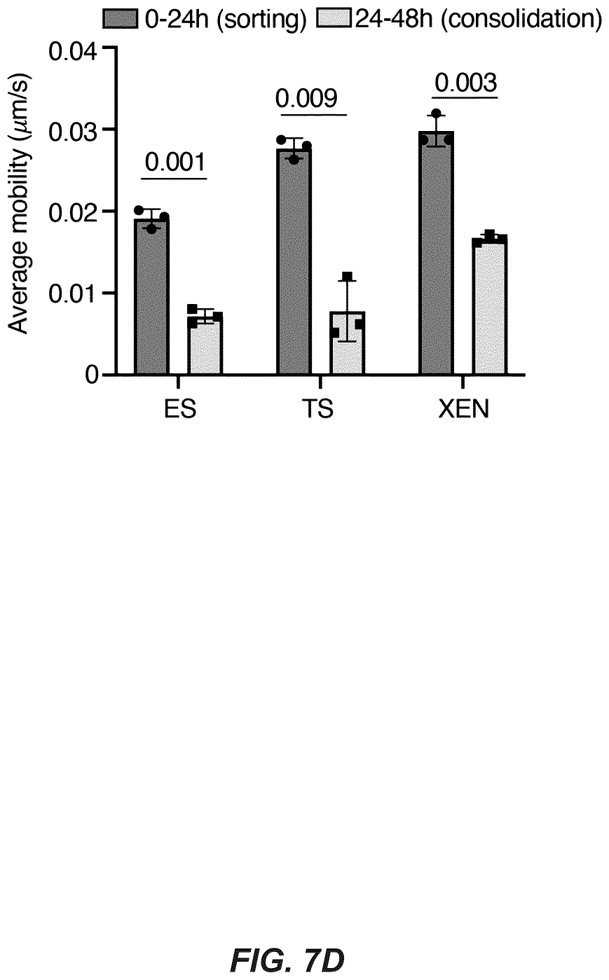

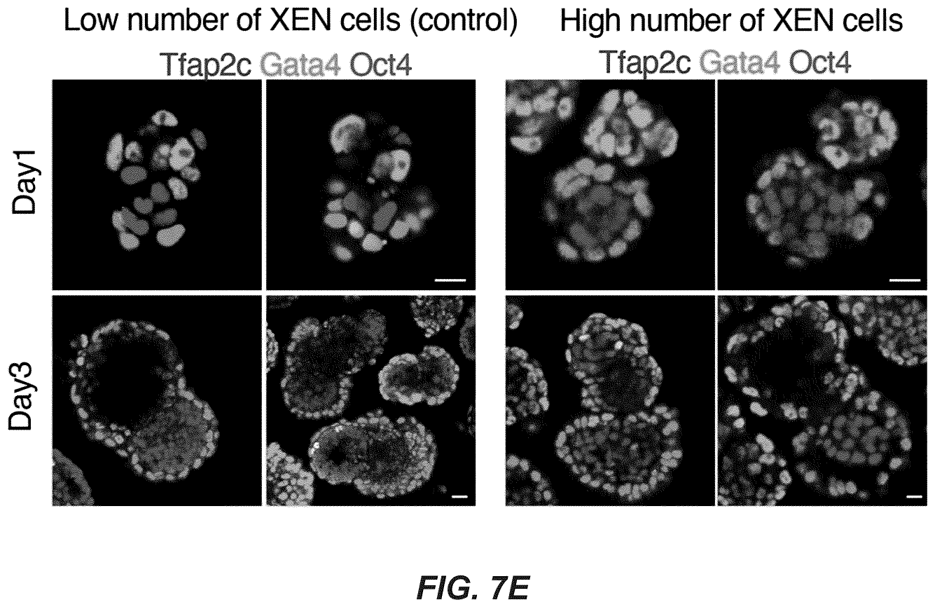

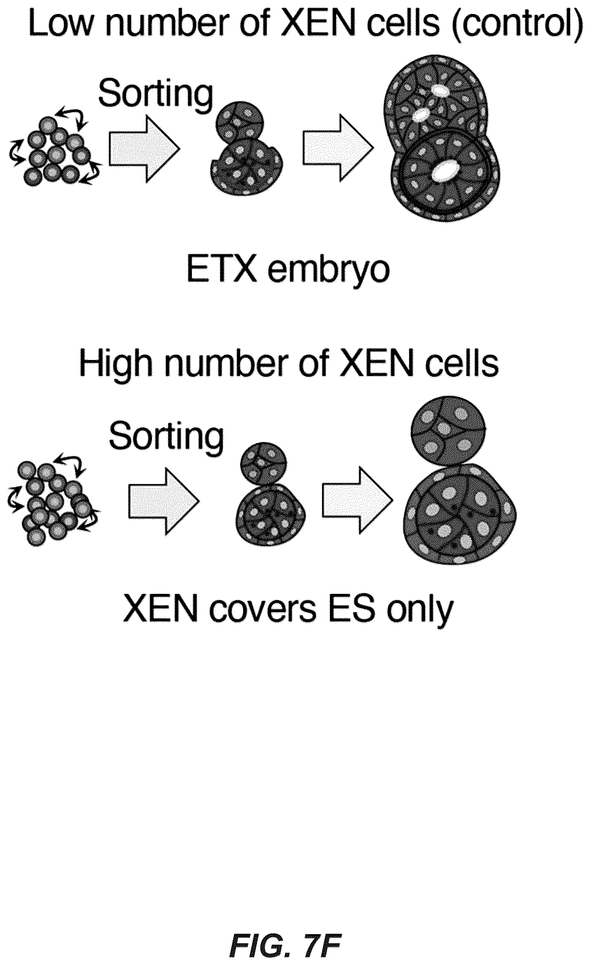

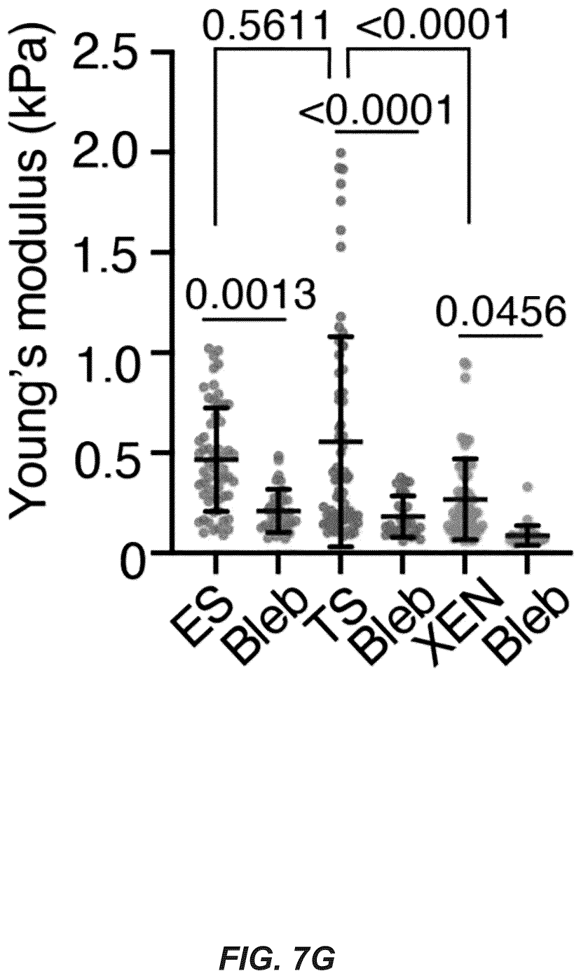

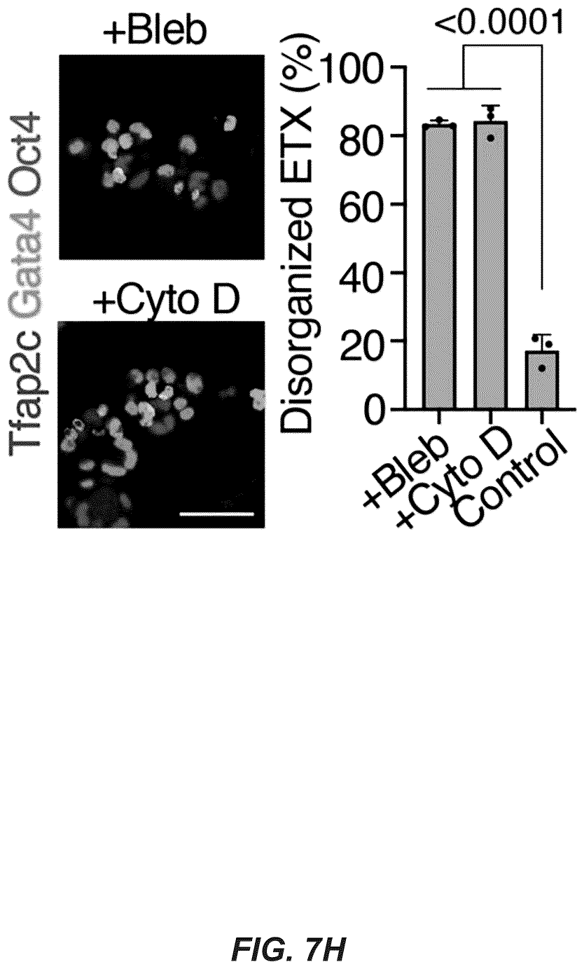

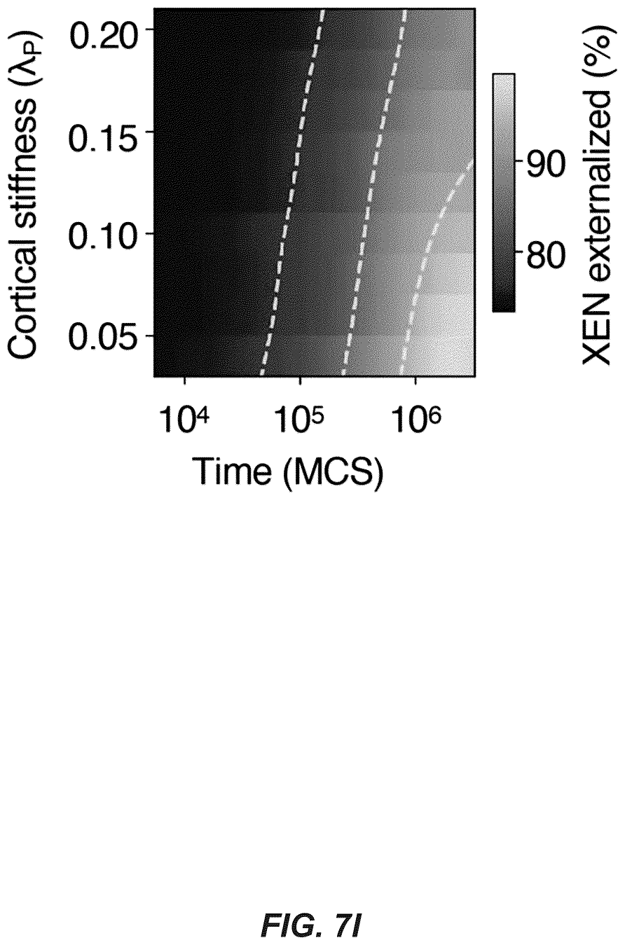

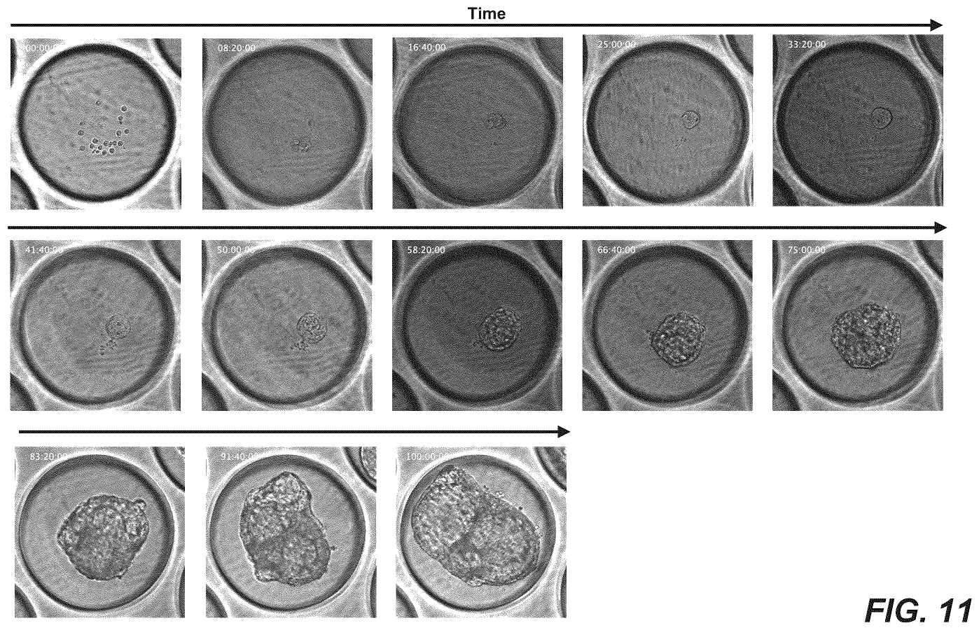

Next, it was determined how the observed cadherin code affects the efficiency of ETX embryogenesis. Single-cell suspensions of ES, TS and XEN cells seeded into microwell plates assembled into multiple structures, of which 15.4% formed ETX structures recapitulating postimplantation embryo morphogenesis (FIG. 3A and FIG. 11). In contrast, 38.2% of structures had more than one ES compartment, 30.8% had more than one TS compartment and 12.8% had mislocalized XEN cells or lacked an outside XEN layer; termed missorted ETX structures (FIG. 3B-FIG. 3D). The proportion of correctly sorted ETX embryos plateaued at 15% after the first day of culture (FIG. 7A). Thus, the three cell types undergo a sorting phase within the first 24 h of seeding before becoming consolidated into compartments. It was hypothesized that cells can no longer sort during the consolidation phase due to their low mobility. To test this, ETX embryo formation was filmed by time-lapse microscopy and cell mobility was tracked (FIG. 7B). This revealed all cell types to be mobile during the cell sorting stage, becoming relatively immobile during the tissue consolidation stage (FIG. 7C-FIG. 7D).

XEN cell sorting fell into a particular two-phase pattern. XEN cells sorted efficiently; over 90% of XEN cells formed a monolayer, first enveloping ES cells and then spreading to cover TS cells. KD of E-cadherin (Cdh1) or K-cadherin (Cdh6), which are co-expressed in XEN cells, reduced the frequency of ETX embryos having a continuous XEN layer (FIG. 3E-FIG. 3F). However, XEN cells with OE of Cdh1 or Cdh6 often missorted within these compartments FIG. 3E-FIG. 3F). Thus, an optimal balance of E-cadherin and K-cadherin contributes to the proper sorting of XEN cells in ETX embryos.

Differential adhesion cannot fully account for the ability of XEN cells to envelop the TS layer because, without being bound by any particular theory, the adhesion force between ES and XEN cells is larger than that between ES and TS cells, and XEN cells were found between the ES and TS compartments in approximately 10% of CPM simulations that sampled these data (FIG. 2I). The discrepancy between the predicted interfacial hierarchy for the sorted configuration in the ETX embryo and the measured differential adhesion force led to the hypothesis that the low number of XEN cells used for making ETX embryos was insufficient to cover all ES cells during the sorting stage. To test this, ES and TS cells were seeded with between five and ten XEN cells and the nascent structures were fixed at days 1 and 3. It was found that low numbers of XEN cells first covered the ES cells only; subsequently, the TS cells enveloped the entire structure (FIG. 7E-FIG. 7F). When approximately ten ES cells were seeded per structure, the XEN cells completely covered the ES cells, thereby excluding TS cells (FIG. 7E-FIG. 7F), consistent with our measurements of differential adhesion.

Previous studies have reported a role for cortical stiffness in cell sorting, particularly in cell externalization, prompting consideration of whether cortical tension may influence the capacity of XEN cells to form their external monolayer. Indeed, the AFM measurements indicated that cortical stiffness is lower in XEN cells than in either TS or ES cells (FIG. 7G). To determine whether differences in cortical stiffness between the different stem cell types of ETX embryos were due to differential actomyosin activity, as in other systems, cortical stiffness in the presence of blebbistatin (a myosin inhibitor) was measured. Blebbistatin reduced the cortical stiffness of both ES and TS cells to the same level as in XEN cells (FIG. 7H). Furthermore, well-sorted ETX embryos treated with either blebbistatin or cytochalasin D (an actin depolymerizer) at day 3 for 24 h (once the primary sorting phase was completed) failed to maintain efficient sorting compared with control ETX embryos (FIG. 7H). Moreover, when well-sorted ETX embryos were treated with either blebbistatin or cytochalasin D for 24 h at day 3, once the primary cell sorting phase was complete, more than 80% and 85% of blebbistatin- and cytochalasin D-treated structures, respectively, failed to maintain sorting compared with 18% of control ETX embryos (FIG. 7H).

To further test the role of cortical stiffness on XEN cell externalization, a CPM was used in which cortical stiffness can be tuned independently. Lower stiffness increased both the sorting efficiency and speed of XEN cell externalization (FIG. 7I), indicating that the softness of XEN cells is important for this event. Together, these data show that, in addition to the differential expression of distinct cadherins, cortical stiffness plays a role in the self-assembly of stem cells into ETX embryos.

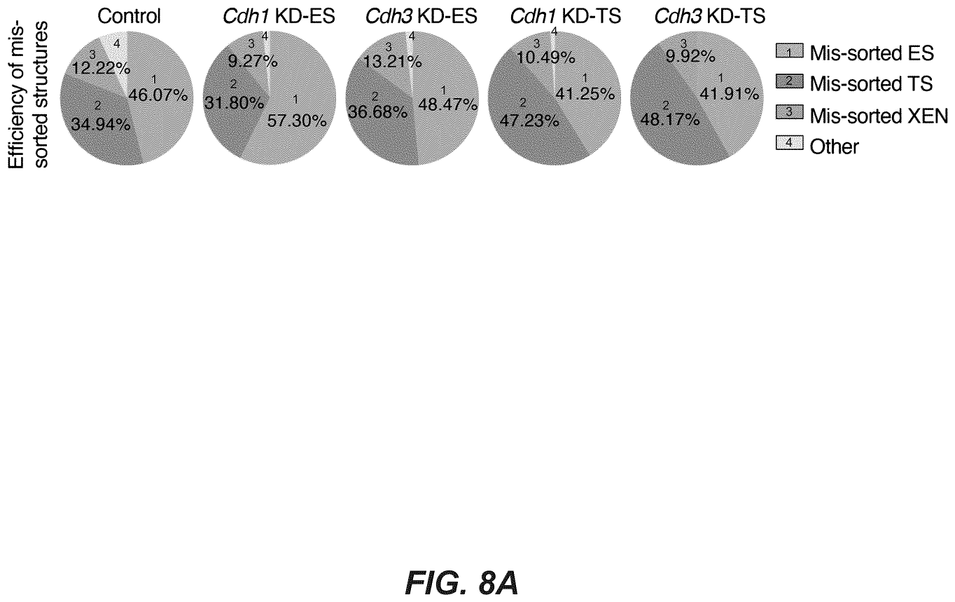

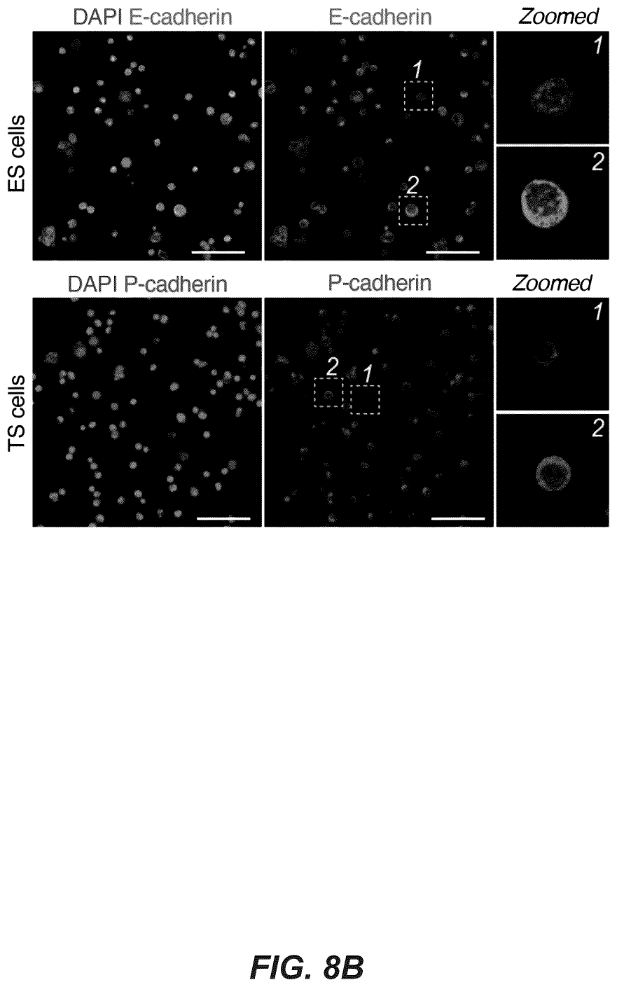

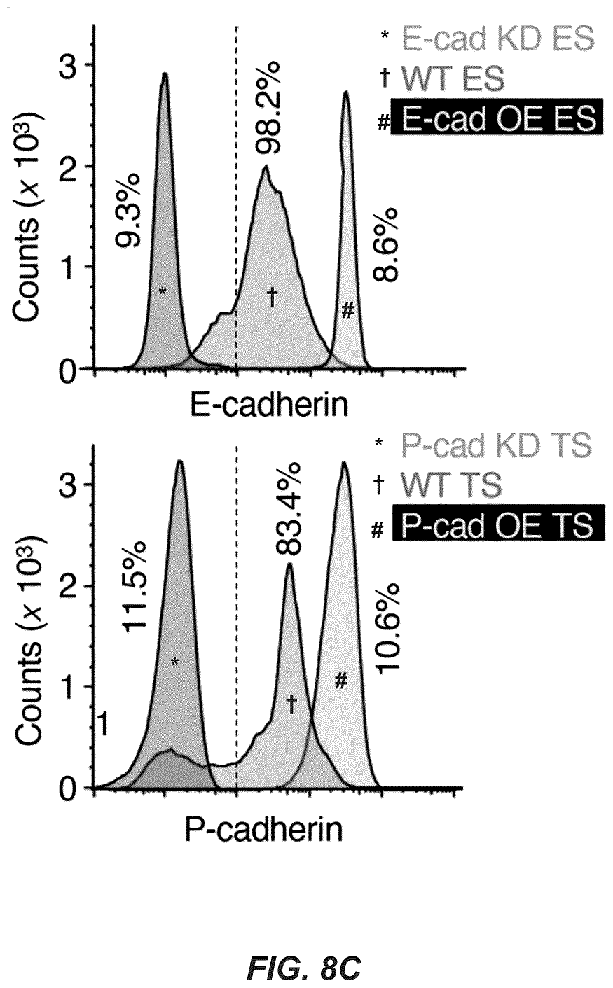

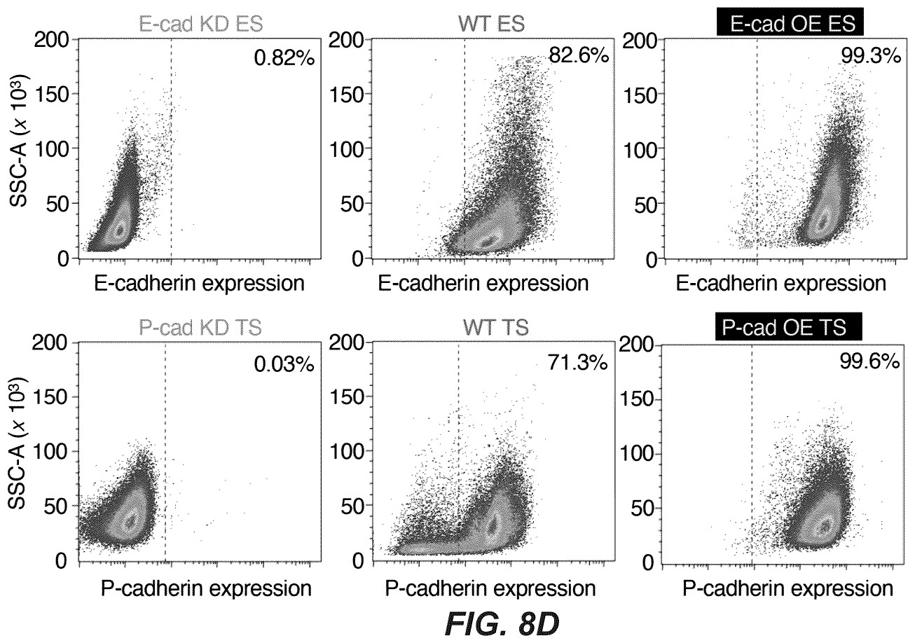

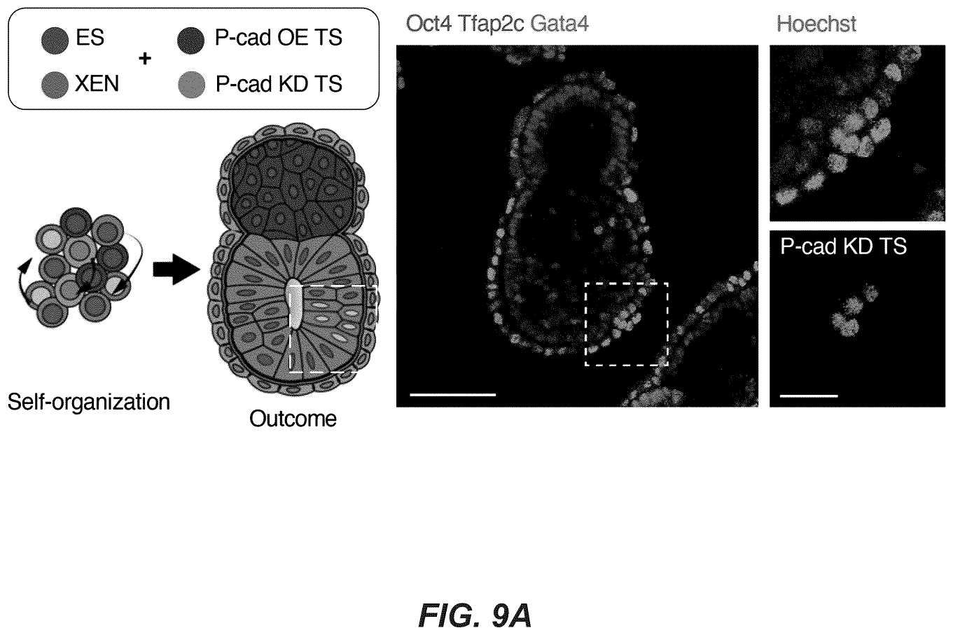

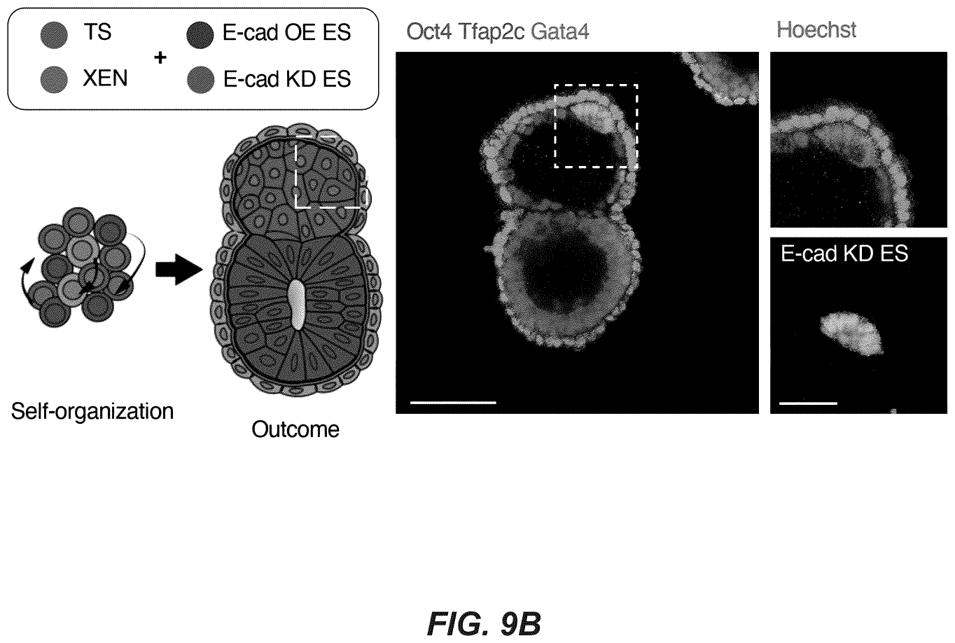

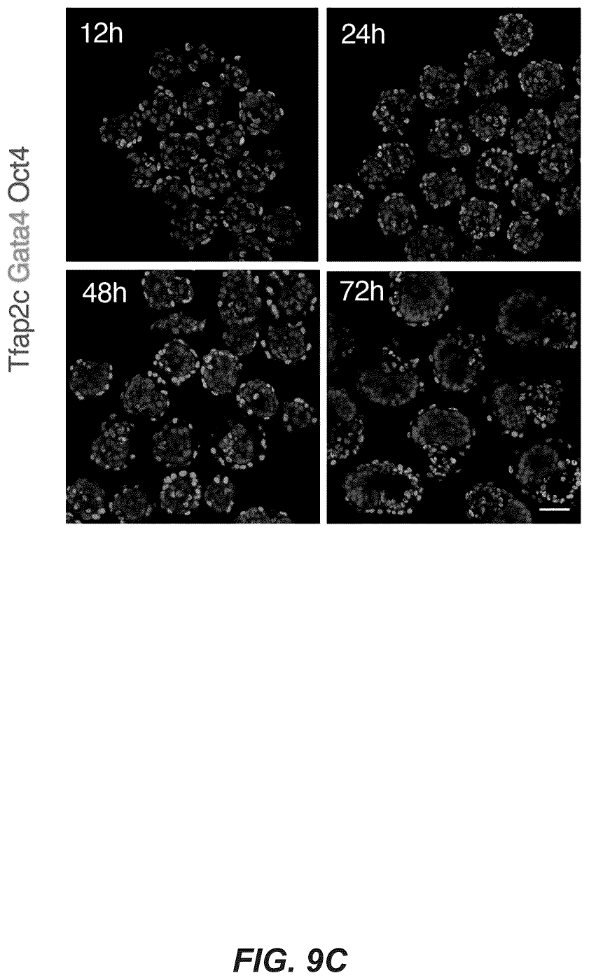

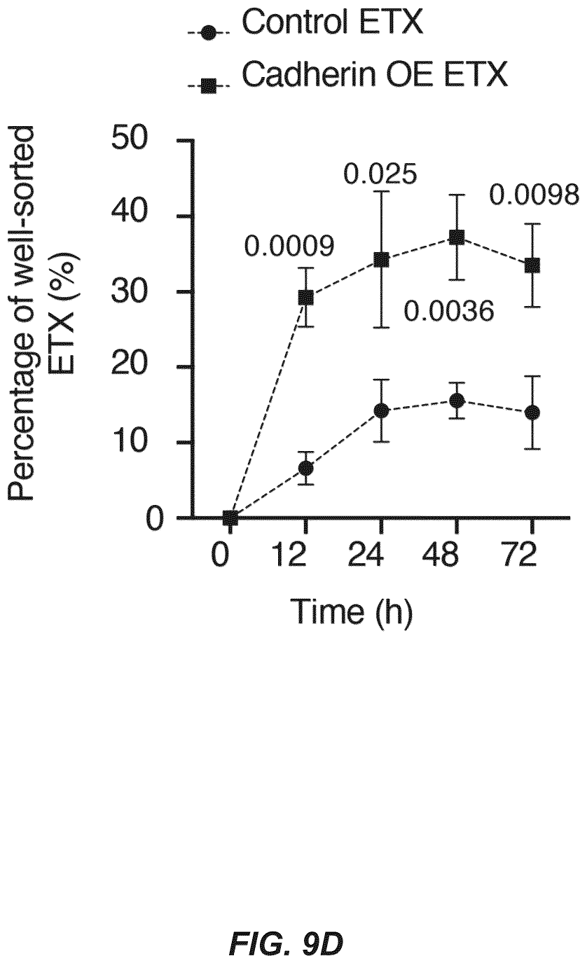

Next, the function of the cadherin code in ES and TS cells during ETX embryo assembly was examined. KD of P-cadherin (Cdh3) in TS cells, but not in ES cells, resulted in TS mislocalization and disrupted ETX embryogenesis. Similarly, KD of E-cadherin (Cdh1) in ES cells, but not TS cells, disrupted ETX embryogenesis. ETX embryo formation still occurred following E-cadherin depletion from TS cells (FIG. 3G and FIG. 8A), indicating that differential expression of P-cadherin between ES cells and TS cells is sufficient to drive their sorting. It was noticed that E-cadherin and P-cadherin showed different levels of expression in individual wild-type ES and TS cells, respectively (FIG. 8B-FIG. 8D). It was considered that subsets of wild-type stem cells with low cadherin expression compromise ETX embryo formation. Indeed, when wild-type ES and XEN cells were combined with either a P-cadherin OE subset or a P-cadherin KD subset of TS cells, P-cadherin KD TS cells mislocalized to the ES compartment. Similarly, when combining wild-type XEN and TS cells with either an E-cadherin OE subset or an E-cadherin KD subset of ES cells, mislocalization of E-cadherin KD ES cells was observed in the TS compartment (FIG. 9A-FIG. 9B). Thus, populations of ES and TS cells with low E-cadherin and low P-cadherin expression, respectively, compromise sorting in ETX embryos. Strikingly, mixing E-cadherin OE ES cells and P-cadherin OE TS cells with wild-type XEN cells increased ETX embryogenesis efficiency by almost threefold from approximately 15% with wild-type stem cells to approximately 42% with the OE cells (FIG. 3H). The time course of the sorting of E-cadherin OE ES cells, P-cadherin OE TS cells and XEN cells revealed that around 30% of these structures were well-sorted 12 h after cell seeding compared with 6.8% of wild-type structures (FIG. 9C-FIG. 9D).

Thus, the sorting rate is increased following cadherin OE, as suggested in simulations. Together, these results show that variable E-cadherin expression in ES cells and P-cadherin expression in TS cells limits the efficiency of ETX embryo formation.

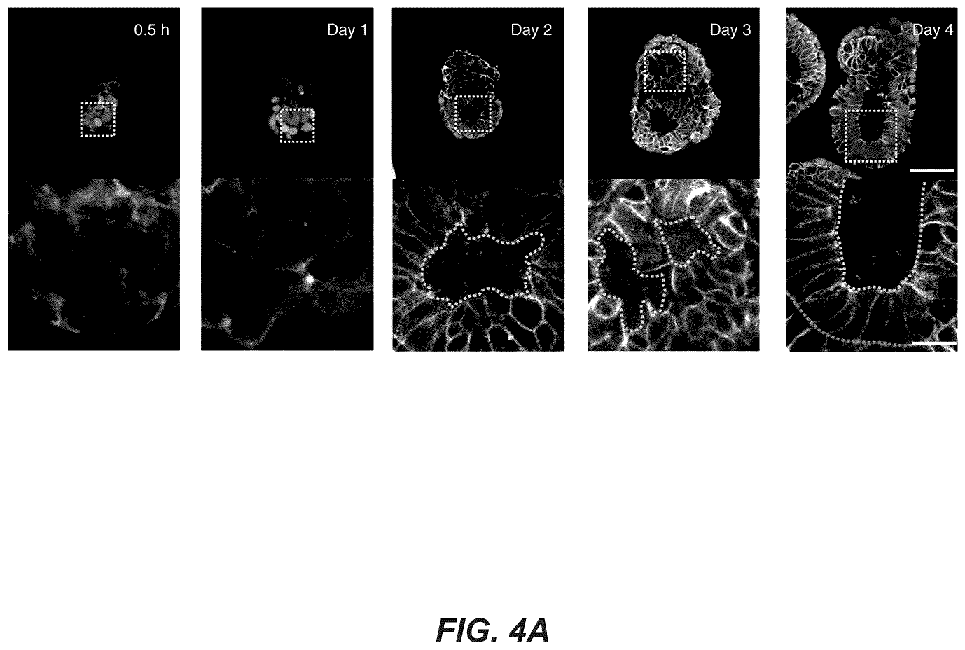

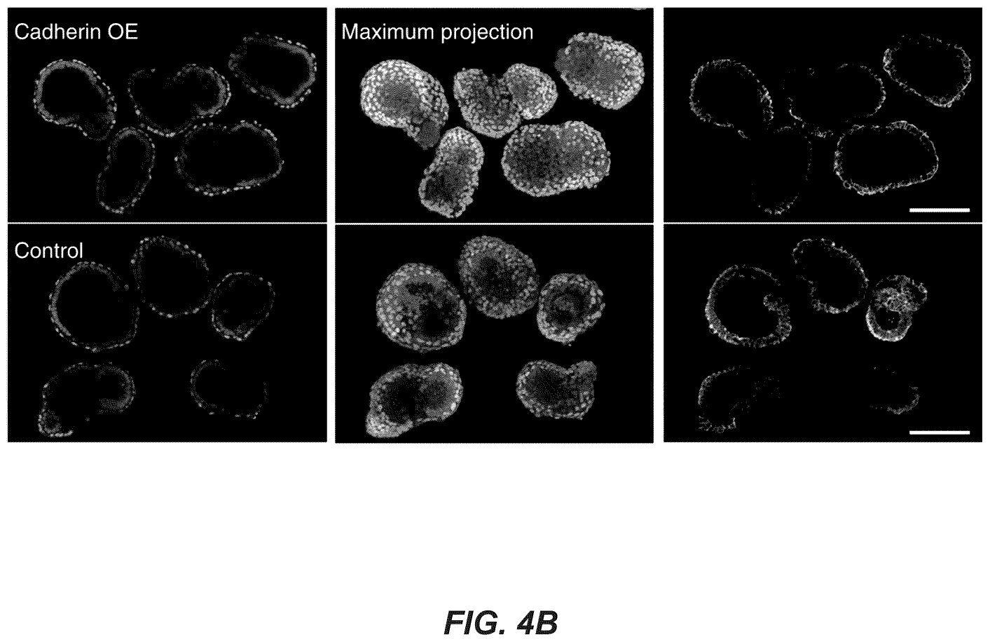

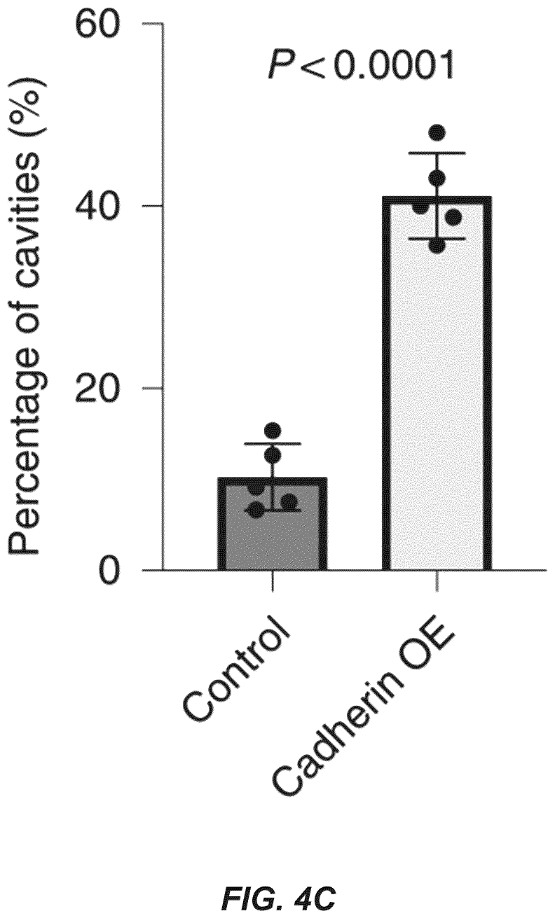

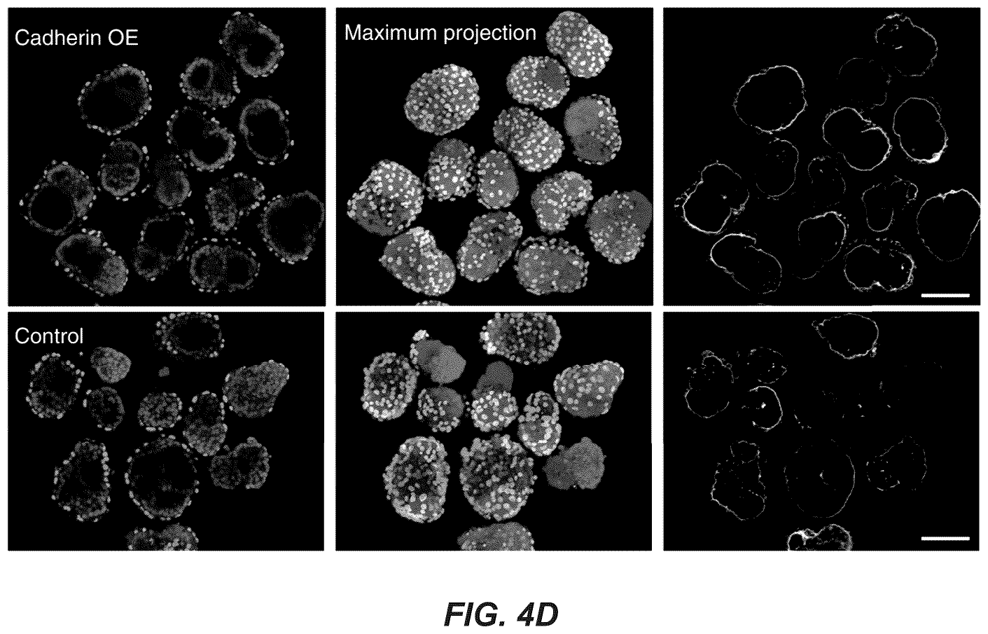

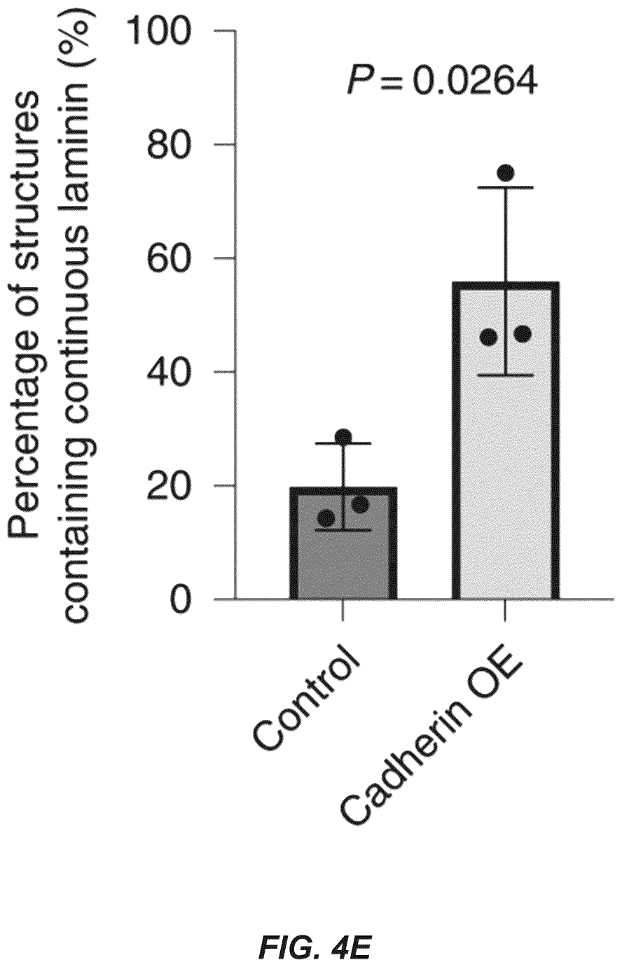

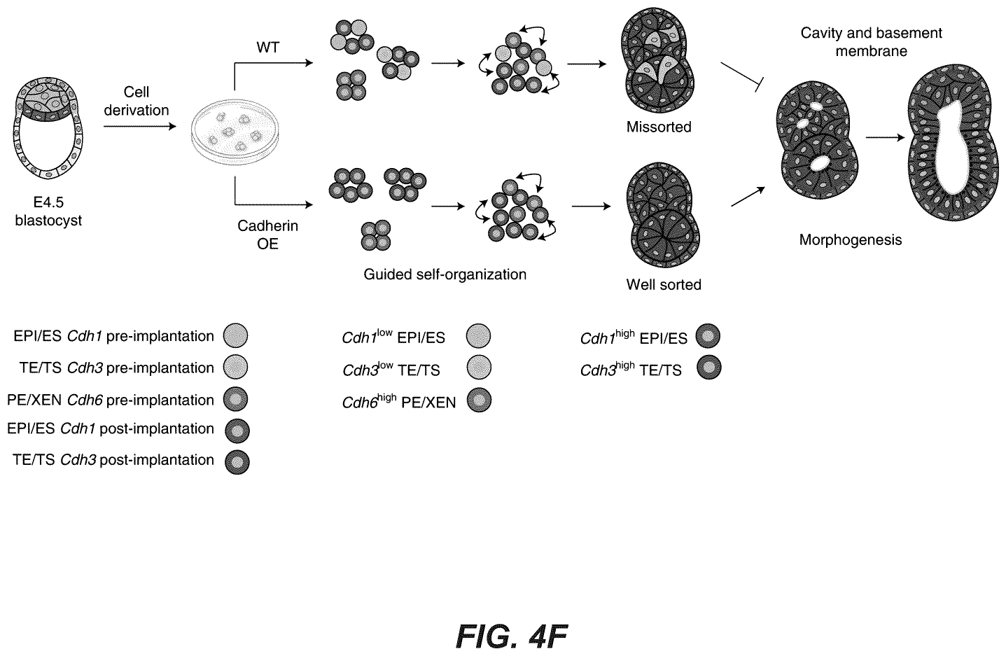

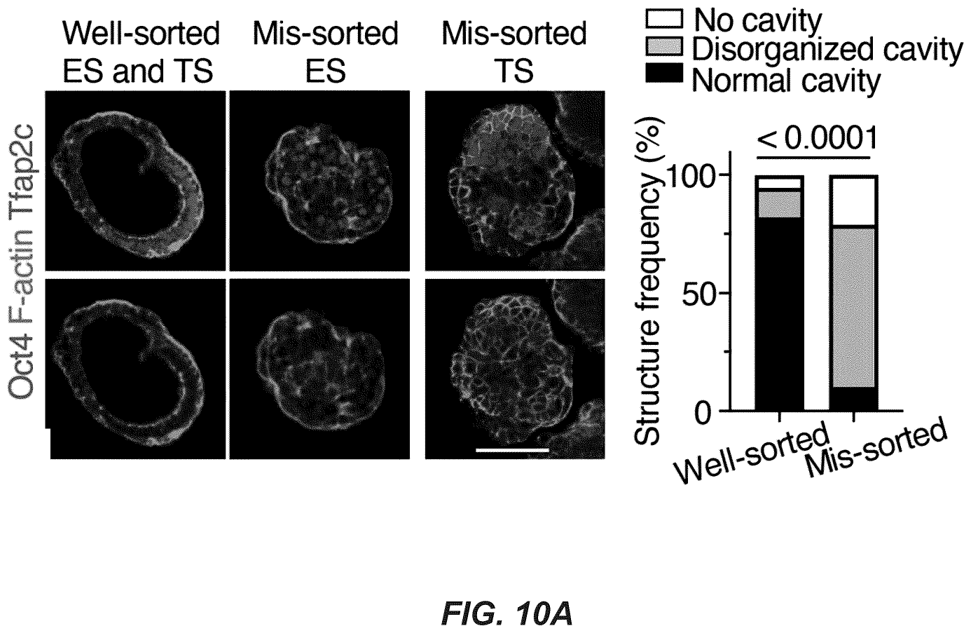

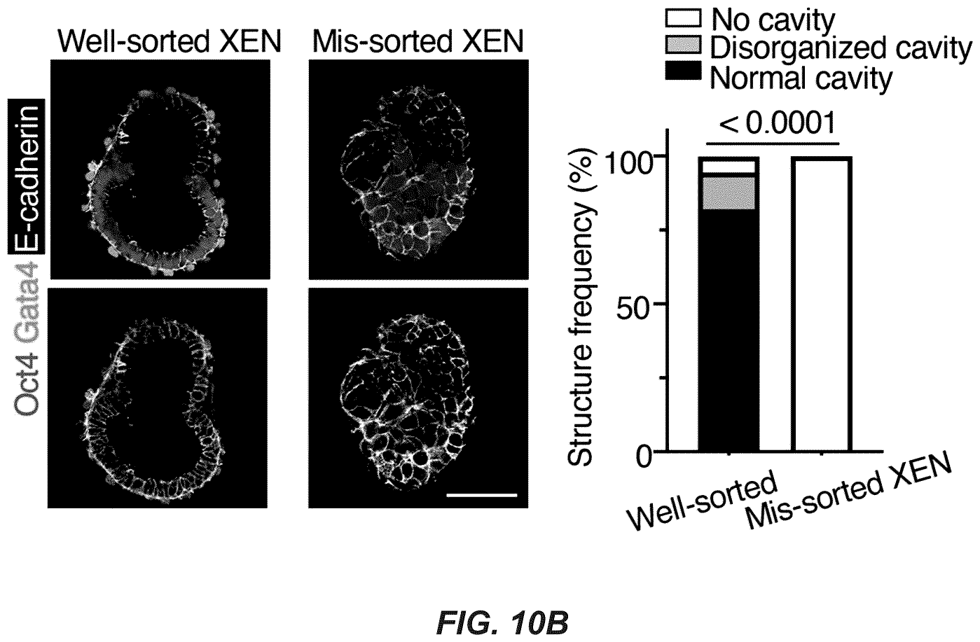

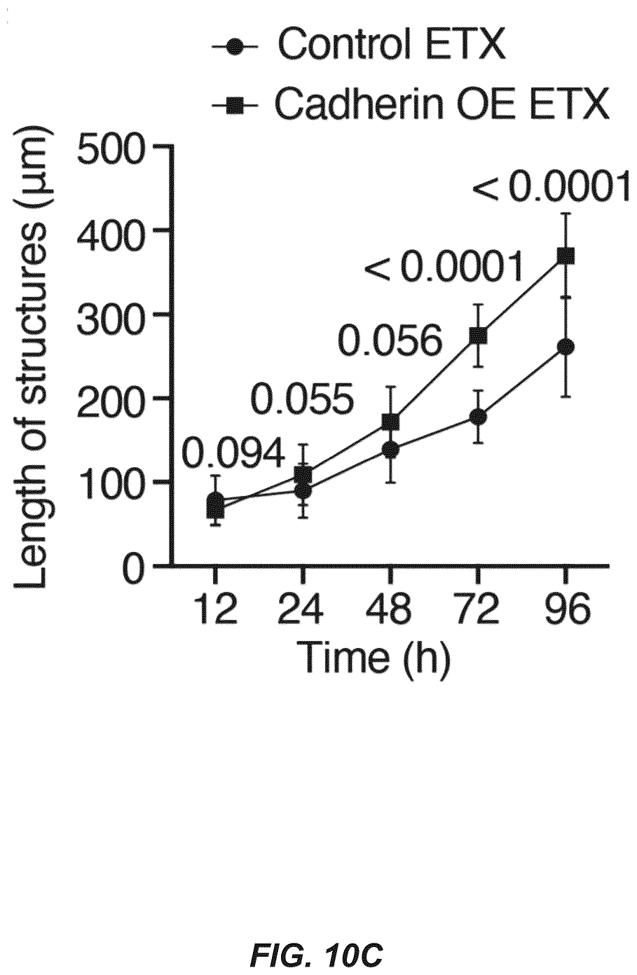

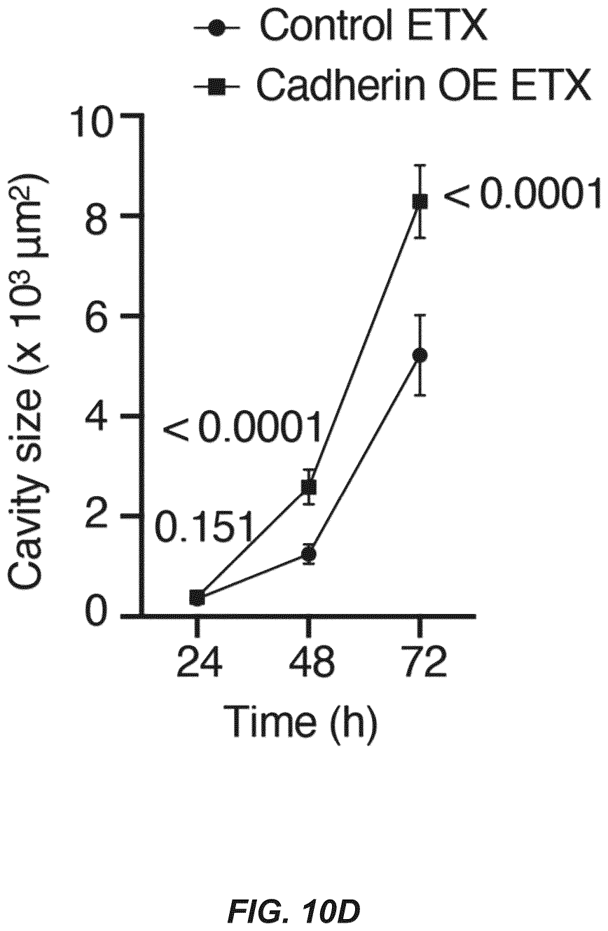

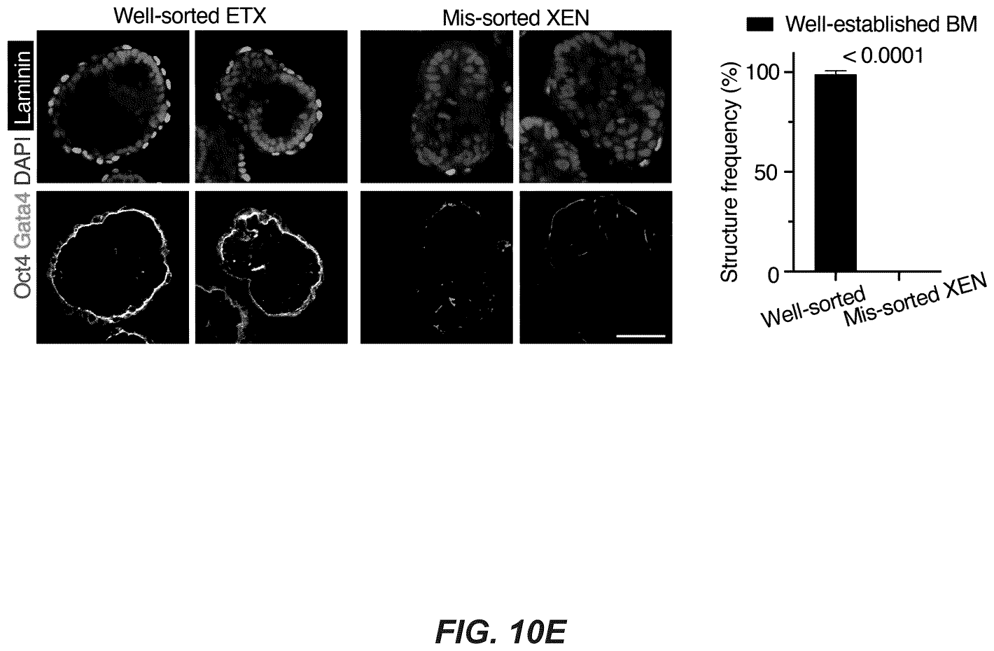

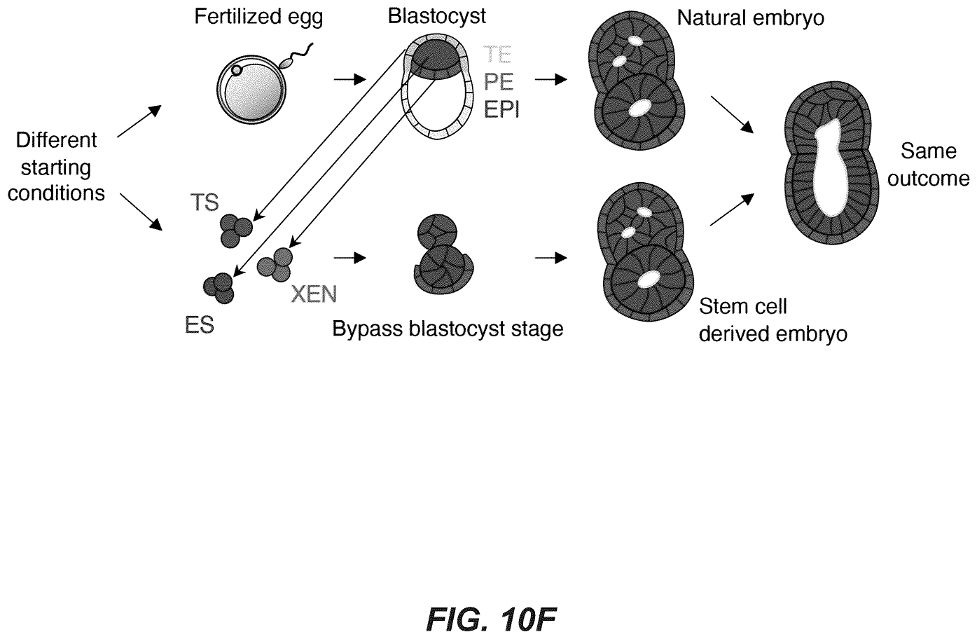

As implantation-stage embryo morphogenesis requires both lumenogenesis and basement membrane formation, it was next determined whether cadherin-enhanced ETX embryo self-organization also improved these events. ETX embryos generated from wild-type stem cells formed a central lumen, corresponding to the lumen of the epiblast rosette at implantation, within the ES compartment by day 2. By day 3, multiple lumens developed in the TS compartment, corresponding to the multiple lumens of the E5.5 extraembryonic ectoderm, and these unified into a single cavity between days 4 and 5, as in natural development by E6.0 (FIG. 4A). Such a single unified cavity formed in over 90% of properly sorted ETX embryos but in fewer than 5% of ETX structures with missorted ES and TS cells (FIG. 10A). Moreover, ETX structures with missorted XEN cells lacked cavities entirely (FIG. 10B). Importantly, proper sorting and amniotic cavity-like formation were observed in only 9% of structures built from wild-type cells but in 40% of structures built from E-cadherin OE ES cells, P-cadherin OE TS cells and wild-type XEN cells (FIG. 4B-FIG. 4C). Moreover, the structures formed from cadherin OE cells, and the cavities within them, were longer than in ETX embryos built from wild-type cells (FIG. 10C-FIG. 10D) after 3 d in culture. Together, this indicates that E- and P-cadherin OE in ES and TS cells, respectively, promotes cavity formation in ETX embryos.

Lumenogenesis requires signaling from the basement membrane, produced by the visceral endoderm. Accordingly, it was found that ETX structures with missorted XEN cells, which lacked a cavity, also failed to establish a basement membrane (FIG. 10E). A continuous laminin-containing basement membrane was detected in 78% of structures built from E-cadherin OE ES cells, P-cadherin OE TS cells and XEN cells (FIG. 4D) but in only 45% of structures made from wild-type ES, TS and XEN cells (FIG. 4E). Thus, elevated expression of E- and P-cadherin in ES and TS cells, respectively, increases the successful formation of basement membrane, lumen and correctly sorted ETX embryos (FIG. 4F).

The methods disclosed herein shed light on the remarkable self-assembly of stem cells into synthetic embryos. As described herein, this requires a cadherin code that, through strong homotypic interactions, sorts ES and TS cells into distinct compartments. In contrast, heterotypic interactions enable XEN cells to first surround ES and then TS cells. Although XEN cells have a cadherin code resembling pre-implantation primitive endoderm, they nevertheless attain the ability to support synthetic post-implantation morphogenesis (FIG. 10F). These differences between natural and ETX embryos highlight, without being bound by any particular theory, the distinct use of common rules between biological development and bioengineering. Synthetic embryo assembly utilizes these codes in a distinct way: XEN cells use the preimplantation code of primitive endoderm to sort in a layer below ES cells, whereas TS cells use the post-implantation code of extraembryonic ectoderm to sort as a cluster above ES cells.

The outcome of cell sorting has been modelled previously by considering cell-specific differences in interfacial energies maximizing the most energetically favorable cell interfaces. Disparity in interfacial energy was considered to reflect adhesion differences, with cadherins being the best-characterized effectors, as espoused in the differential adhesion hypothesis (DAH). In accord, it is disclosed herein that cell sorting is driven in ETX embryos by increased cadherin-mediated homotypic interactions in relation to heterotypic interactions. The later development of the differential interfacial tension hypothesis (DITH) invoking the role of differential cortical tension in sorting resonates with the present disclosure on XEN cell externalization in self-assembly. Together, the present disclosure supports the balance between adhesion and tension (DAH versus DITH) as in biophysical models of cell sorting. However, in some embodiments, incomplete ES-TS sorting still results in local order, emphasizing a need for global-scale sorting to fully recapitulate natural morphogenesis. DAH and DITH only account for local sorting to form homotypic clusters of ES and TS cells, as seen even in missorted structures. For complete sorting, ETX embryos must escape from locally correct neighborhoods within globally incorrect patterns to explore alternative conformations. If cells remain in local minima before cell sorting is complete, structures will remain mis sorted.

The importance of the cell type-specific cadherin code is illustrated by the finding that established wild-type stem cell lines show heterogeneous cadherin expression, with some subsets below the threshold required to support proper sorting. Elevating E-cadherin in ES cells and P-cadherin in TS cells substantially improves ETX embryogenesis efficiency (FIG. 4F). This identifies a broader challenge in synthetic biology; namely, characterizing and ameliorating the impacts of heterogeneity in stem cell lines—factors that remain for the most part undefined but reported. Such heterogeneity might affect, in some embodiments, the distribution of cell-cell cohesive properties within the same cell population, confounding the hierarchy of interactions necessary to drive self-organization of other organoid structures. Thus, the principles of self-organization described herein can overcome these challenges and provide a means for increasing the efficiency of formation of different types of organoids by modulating interactions and the physical properties of cells.

### Methods

### Cell Culture

All cells were cultured at 37° C. in 20% O₂ and 5% CO₂ and passaged once they had reached 70% confluency. Cells were tested weekly for Mycoplasma contamination by PCR.

ES cells were cultured on a 0.1% gelatin-coated plate in N2B27 medium with 1 μM MEK inhibitor PD0325901, 3 μM GSK3 inhibitor CHIR99021 and 10 ng/ml leukaemia inhibitory factor. The N2B27 medium comprised a 1:1 mix of Dulbecco's modified Eagle medium (DMEM)/F12 (21331-020; Thermo Fisher Scientific) and Neurobasal-A (10888-022; Thermo Fisher Scientific) media supplemented with 0.5% vol/vol N2 (17502048; Thermo Fisher Scientific), 1% vol/vol B27 (10889-038; Thermo Fisher Scientific), 100 μM β-mercaptoethanol (31350-010; Thermo Fisher Scientific), 1% vol/vol penicillin-streptomycin mix (15140122; Thermo Fisher Scientific) and 1% vol/vol GlutaMAX (35050-061; Thermo Fisher Scientific).

TS cells (wild type) were cultured on mitomycin C (M4287; Sigma-Aldrich)-treated CF1 mouse embryonic fibroblasts (MEFs) in TSF4H medium with RPMI 1640 (M3817; Sigma-Aldrich) containing 20% foetal bovine serum (FBS; 35-010-CV; Thermo Fisher Scientific), 2 mM 1-glutamine, 0.1 mM β-mercaptoethanol, 1 mM sodium pyruvate, 1% penicillin-streptomycin (M7167; Sigma-Aldrich), 25 ng ml−1 FGF4 (5846-F4; R&D Systems) and 1 μg/ml heparin (H3149; Sigma-Aldrich).

TS cells (Cdh3 OE) were cultured on 10 μg/ml laminin-coated plates in TX medium, with 50 ng/ml IL11 (50117-MNCE; Sino Biological), 50 ng/ml activin (Qk001-ActA-100; Qkine), 25 ng/ml Bmp7 (PeproTech; 120-03P-10 μg), 5 nM lysophosphatidic acid (Santa Cruz Biotechnology; sc-201053) and 200 nM 8Br-cAMP (B 007-500; BIOLOG Life Science Institute). TX medium was made from a 1:1 mix of DMEM and F12 media (21331-020; Thermo Fisher Scientific) with 19.4 μg/ml insulin (342106; Sigma-Aldrich), 64 μg/ml 1-ascorbic-acid (A4403; Sigma-Aldrich), 14 ng/ml sodium selenite (S5261; Sigma-Aldrich), 543 μg/ml sodium bicarbonate (S5761; Sigma-Aldrich), 10.7 μg/ml holo-transferrin (T4132; Sigma-Aldrich), 1% penicillin-streptomycin (M7167; Sigma-Aldrich), 25 ng/ml FGF4 (5846-F4; R&D Systems), 2 ng/ml TGF-β1 (100-21 C; PeproTech) and 1 μg/ml heparin (H3149; Sigma-Aldrich). After selection, Cdh3 OE TS cells were cultured on MEF in TSF4H medium.

XEN cells were cultured on gelatin-coated plates in 70% MEF-conditioned IDG medium (c-IDG). c-IDG medium comprised DMEM (21969; Gibco) containing 12.5% FBS (35-010-CV; Thermo Fisher Scientific), 2 mM GlutaMax (35050-038; Gibco), 0.1 mM 2-mercaptoethanol (31350-010; Gibco), 0.1 mM nonessential amino acids (11140-035; Gibco), 1 mM sodium pyruvate (11360-039; Gibco), 0.02 M HEPES (15630080; Gibco) and 1% penicillin-streptomycin (15140122; Gibco).

MEF cells were cultured on gelatin-coated plates in DMEM medium (41966; Thermo Fisher Scientific) supplemented with 15% FBS (35-010-CV; Thermo Fisher Scientific), penicillin-streptomycin (15140122; Thermo Fisher Scientific), GlutaMAX (35050061; Thermo Fisher Scientific), MEM nonessential amino acids (11140035; Thermo Fisher Scientific), sodium pyruvate (11360070; Thermo Fisher Scientific) and 100 μM β-mercaptoethanol (31350-010; Thermo Fisher Scientific).

### Mouse Embryos

Mice were maintained according to national and international guidelines. All experiments were regulated by the Animals (Scientific Procedures) Act 1986 Amendment Regulations 2012 following ethical review by the University of Cambridge Animal Welfare and Ethical Review Body. Experiments were approved by the Home Office. Animals were inspected daily and those that showed health concerns were culled by cervical dislocation. Six-week-old female CD-1 mice were used in all of the animal experiments. All experimental mice were free of pathogens and were on a 12 h light/12 h dark cycle, with unlimited access to water and food. The temperature in the facility was controlled and maintained at 21° C.

### Generation of Cell Lines

The experiments were performed using mouse E14 wild-type ES cells Cdh1 and Cdh6 OE ES cells were generated from E14 wild-type ES cells, the Cdh3 OE TS cells were generated from wild-type TS cells and the Cdh1 and Cdh6 OE XEN cells were generated from wild-type XEN cells (see below).

To generate cadherin OE ES, TS or XEN cell lines, 0.5 μg of a super piggyBac transposase expression vector (PB210PA-1; System Biosciences) and 2 μg Cdh1-pHygro, Cdh3-pHygro or Cdh6-pHygro plasmid were co-transfected into cells using Lipofectamine. Cells were passaged for 24 h after transfection and subjected to selection in medium containing 50 μg ml−1 hygromycin (10687010; Thermo Fisher Scientific) for 1 week. A similar approach was used to generate the stable nuclear reporter H2B-RFP XEN cell line.

### Cloning

Cloning procedures were performed using Gateway technology (Thermo Fisher Scientific). The fragment of interest (Cdh1, Cdh3 or Cdh6) was amplified by PCR to introduce attB sites. These fragments were cloned into the pDONR221 vector (a gift from J. Silva) using BP clonase II (11789020; Thermo Fisher Scientific). The fragment of interest (Cdh1, Cdh3 or Cdh6) was subcloned into a pHygro vector containing a hygromycin resistance cassette for expression in stem cells. The recombination reaction was carried out using LR Clonase II (11791100; Thermo Fisher Scientific).

PiggyBac-based expression plasmids for Cdh1, Cdh3 or Cdh6 were generated by PCR amplification of the respective genes in pENTR-Cdh1 (49776; Addgene), Cdh3 (Myc-DDK tagged) (MR227345; OriGene Technologies) or Cdh6 (mouse-tagged ORF clone) (MG222740; OriGene Technologies) with the oligos listed in Table 5.

### Small Interfering RNA

Cells were transfected with 25 nM small interfering RNA (siRNA) directed against Cdh1 (1027418-S100946631), Cdh3 (1027418-S102666440) or Cdh6 (1027418-SI00946967) (Qiagen) siRNA or with control scrambled siRNA (Qiagen) using Lipofectamine 3000 transfection reagent according to the manufacturer's instructions. Cells were harvested at 72 h post-transfection and assayed by quantitative PCR (qPCR).

### Flow Cytometry Analysis

Single ES and TS cell suspensions were collected, fixed in 4% paraformaldehyde and permeabilized for 30 min at room temperature using 0.3% Triton X-100 and 0.1% glycine. Cells were then incubated with anti-E-cadherin (1:200; 13-1900; Thermo Fisher Scientific) or P-cadherin antibody (1:100; sc-1501; Santa Cruz Biotechnology) for overnight incubation at 4° C. in blocking buffer (phosphate-buffered saline (PBS) solution plus Tween 20) (PBST) containing 10% FBS). Cells were washed twice in PBST and then incubated with secondary antibody (1:500 dilution) in blocking buffer at room temperature for 1-2 h. Cells were then analysed by quantitative flow cytometry (BD Biosciences) and the intensity profiles of E- and P-cadherin were plotted using FlowJo software (version 10.7.1) (https://www.flowjo.com).

### Cell Doublets Experiment

To measure cell-cell contact angles, 1200 dissociated ES, TS or XEN cells were mixed in pairs and seeded onto AggreWell plates (34411; STEMCELL Technologies) pretreated with rinsing solution (07010; STEMCELL Technologies). Cells were centrifuged at 100 g for 3 min. After 1 h incubation at 37° C., cells were collected and fixed for immunostaining.

### Cadherin-Coated Surface Preparation

To measure the adhesion forces between cells and cadherin-coated surfaces, briefly, gold-coated glass cover slips (AU.0100; Platypus) were cleaned in argon plasma for 30 s and subsequently functionalized by immersion in thiol solution (P50757; Sigma-Aldrich) for 16 h, then rinsed with EDTA-buffer (15575-020; Thermo Fisher Scientific) to remove excess thiol. The cover slips were subsequently incubated with 10 μg/ml recombinant E-cadherin (8875-EC-050; R&D Systems) or P-cadherin (761-MP-050; R&D Systems) for 12 h at 4° C. Before making force measurements, the cadherin-coated surfaces were washed with HEPES (15630106; Thermo Fisher Scientific) and activated by incubation in the same buffer for 30 min.

### Adhesion Force Measurement

Cell-cell or cell-cadherin adhesion forces were measured using an atomic force microscope (Bruker NanoScope) coupled to a confocal microscope (TCS SP5II; Leica). Tipless silicon nitride cantilevers were V shaped, with nominal spring constants (60 pN nm−1; NP-0; Veeco Instruments). The atomic force microscope cantilevers were plasma cleaned before functionalization with concanavalin A. The system was calibrated in cell-free medium at 37° C. before each experiment by measuring the deflection sensitivity on a glass surface, allowing the cantilever spring constant to be determined in situ. Before loading the sample, the sample stage movement was calibrated using NanoScope software (version 6.13). Before measurements, cells were dissociated with TrypLE (12604013; Thermo Fisher Scientific) and resuspended in HEPES-buffered cell culture medium (15630056; Thermo Fisher Scientific). Cell suspensions were loaded into the atomic force microscope sample chamber and a single cell was captured by pressing the cantilever onto the cell with a contact force of 500 pN for 1 min. The cell was lifted from the surface and allowed to establish firm adhesion on the cantilever for 5 min. To measure the cell-cell adhesion force, the captured cell was lowered to contact with another single cell cultured on a gelatin-coated glass-bottom Petri dish (FD35; WPI).

To measure the cell-cadherin adhesion force, the captured cell was lowered to contact cadherin-coated cover slips. The approach and retraction speeds were kept constant at 10 μm/s with a contact force of 2 nN. Three force curves were acquired for each cell. The captured cell was left to recover for 3 min between different adhesion force measurement cycles before it was adhered to the surface in a different position. Before and after every single measurement, it was checked that the probing cell remained on the cantilever by direct observation. Maximal cell adhesion forces as well as the single rupture force step height were extracted from retrace curves using JPK IP software.

### Cell Cortical Stiffness Measurements

The stiffness of cells was measured using an atomic force microscope (Bruker NanoScope) coupled to a confocal microscope (TCS SP5II; Leica), as described previously. The point-and-shoot procedure (NanoScope software; Bruker) was used to measure cell stiffness. All cells were kept in CO₂-independent cell culture medium during the measurement.

A fluorescent 10 μm polystyrene bead (Invitrogen) was glued to silicon nitride cantilevers with nominal spring constants of 0.06 N m−1 (NP-S type D; Bruker). Indentations were performed using the single force option with a total indentation depth of 50-100 nm. To obtain cell stiffness values from force curves, PUNIAS software was used. Multiple force displacement curves (at five different locations) were fitted to the Hertz model to calculate cell cortical stiffness (Young's modulus).

### Stem Cell-Derived ETX Embryo Generation

Approximately 6000-7000 ES cells, 15,000-19,000 TS cells and 5000-6000 XEN cells were added dropwise into AggreWell plates having 1200 microwells in one well (34411; STEMCELL Technologies). The microwells were treated with rinsing solution (07010; STEMCELL Technologies). Cells were centrifuged at 100 g for 3 min. 1.5 ml c-IDG medium containing 7.5 nM ROCK inhibitor (72304; STEMCELL Technologies) was added dropwise to each well. On the following day (day 1), 1 ml medium was removed gently from each well and replaced with 1 ml fresh c-IDG medium without ROCK inhibitor. This step was repeated once to fully remove the ROCK inhibitor. On day 2, 1 ml c-IDG medium was replaced with 1 ml fresh medium. On day 3, the media was replaced with IVC1 medium. IVC1 medium comprises advanced DMEM/F12 (21331-020; Gibco) supplemented with 20% (vol/vol) FBS, 2 mM GlutaMax, 1% vol/vol penicillin-streptomycin, 1×ITS-X (51500-056; Thermo Fisher Scientific), 8 nM β-estradiol, 200 ng ml−1 progesterone and 25 mM N-acetyl-1-cysteine.

### Immunofluorescence

Natural embryos, stem cell-derived structures or stem cells were fixed in 4% paraformaldehyde (15710; Electron Microscopy Sciences) for 20-30 min at room temperature, washed twice in PBST (containing 0.05% Tween 20) and permeabilized for 30 min at room temperature in 0.3% Triton X-100 and 0.1% glycine. Primary antibody incubation was performed overnight at 4° C. in blocking buffer (PBST containing 10% FBS). The following day, samples were washed twice in PBST and then incubated with secondary antibody (1:500) in blocking buffer at room temperature for 1-2 h. Embryos were transferred to PBST drops in oil-filled optical plates before confocal imaging.

The following primary antibodies were used: Tfap2c (1:200; AF5059; R&D Systems), Brachyury (1:200; AF2085; R&D Systems), Gata4 (1:500; 36966; Cell Signalling Technology), Laminin (1:500; L9393; Sigma-Aldrich), Oct4 (1:500; sc-5279; Santa Cruz Biotechnology), E-cadherin (1:200; 13-1900; Thermo Fisher Scientific) and P-cadherin (1:100; sc-1501 (Santa Cruz Biotechnology) or MS-1741 (Fisher Scientific)). The following secondary antibodies from Thermo Fisher Scientific were used: Alexa Fluor 488 Donkey anti-Mouse (1:500; A-21202), Alexa Fluor 488 Donkey anti-Goat (1:500; A-11055), Alexa Fluor 488 Donkey anti-Rat (1:500; A-21208), Alexa Fluor 568 Donkey anti-Rabbit (1:500; A-10042), Alexa Fluor 568 Donkey anti-Mouse (1:500; A-10037), Alexa Fluor 647 Donkey anti-Goat (1:500; A-21447) and Phalloidin (1:200; A30104). Detailed information of the used antibodies is provided in Table 3.

RNA Extraction and Real-Time qPCR

Total RNA was extracted from cells using TRIzol Reagent (15596-026; Invitrogen). Real-time qPCR was performed with SYBR Green PCR Master Mix (4368708; Applied Biosystems) and StepOnePlus Real-Time PCR System (Applied Biosystems). The fold change in mRNA expression was determined using the ΔΔCt method with Gapdh as an endogenous control. For the qPCR primers used, see Table 4.

scRNA-seq Sample Preparation and Dissociation

Natural and ETX embryos were transferred to Falcon tubes, washed with PBS and incubated in TrypLE Express (12604013; Gibco) for 15 min at 37° C. to dissociate them into single cells. If clumps remained, the incubation was extended for an additional 5 min at 37° C. and the sample pipetted further. Samples were filtered to remove large clumps, centrifuged at 200 g for 5 min and resuspended in PBST (containing 0.02% Tween 20) and then processed for encapsulation, as previously reported. For E5.5 embryos, one litter of 12 embryos was dissociated together. A total of 15 ETX embryos were dissociated for sequencing. Cells in culture were dissociated into a single-cell suspension using TrypLE Express (12604013; Gibco) and multiplexed using MULTI-seq lipid-modified oligos before running on two 10X Genomics lanes using single-cell 3′ version 3 reagents as reported.

scRNA-seq Analysis

A previously submitted and filtered scRNA-seq dataset comprising the ETX and natural embryos was downloaded from the Gene Expression Omnibus repository (GSE161947). The count matrix was loaded into Seurat version 3, the fraction of counts mapping to mitochondrial genes was computed and the object was then log-normalized to a scale factor of 10,000. The 2,000 most variable genes were computed, the object was scaled and the percentage of mitochondrial counts was regressed out. Dimensional reduction was performed with principal component analysis and the data were projected on a uniform manifold approximation and projection low-dimensional space using 20 principal components. The embryonic, endoderm and trophectoderm lineage identity was pooled from previous annotations, corresponding to clusters with high Dnmt3b, Gata4 and Lamb1 and Cdx2 and Gata2 expression levels, respectively. The average expression was computed using the average expression function and the latter was log₂ normalized. The expression levels of cadherins and protocadherins were subsequently plotted on a heatmap. The uniform manifold approximation and projection plots were directly plotted using the methodology described recently after keeping the ETX and natural embryo samples only and re-computing the neighbourhood graph (five neighbours and 30 principal components; code at https://github.com/fhlab/scRNAseq_inducedETX).

### Time-Lapse Imaging

To perform time-lapse imaging, cells were seeded on Gri3D PEG-hydrogel dishes with glass bottoms (SUN Bioscience) and imaged under a spinning-disc microscope (3i) with a Zeiss EC Plan-NEOFLUAR 20×/0.5 objective in a humidified chamber at 37° C. with 5% CO₂. The structures were imaged every 5-10 min by collecting image stacks of 10 μm z-planes. Images were processed using SlideBook 5.0 (3i). Raw data were processed using the open-source image analysis software Fiji. For single-cell tracking, Imaris image analysis software (Bitplane) was used.

### Quantification and Statistical Analysis

### Criteria for Selecting ETX Embryos

Egg cylinder structures with one TS-derived compartment and one ES-derived compartment, covered by an outside XEN-derived visceral endoderm-like monolayer were considered to be well-sorted ETX embryos for analysis. Structures that did not fulfil these criteria were considered to be missorted ETX structures. Structures containing all three types of cells were collected and counted for quantification.

### Image Data Acquisition, Processing and Quantification

Fluorescence images were acquired using an inverted Leica SP8 confocal microscope (LEICA software LAS X; Leica Microsystems) with a Leica FLUOTAR VISIR 25× or 40× objective.

Images were acquired with 0.5-3.0 μm z-separation. To screen entire structures, the tile-scan imaging mode with automatic image stitching of the SP8 confocal microscope was used. All images were analyzed and processed using Fiji software (http://fiji.sc). For digital quantifications and immunofluorescence signal intensity graphs, laser power and detector gain were maintained constant to permit quantitative comparisons of different experimental conditions within a single experiment.

To evaluate cell-cell contact angles in 3D ETX and natural embryos, ImSAnE was employed to extract planes of the embryos from 3D stacks of E-cadherin and unroll them into a two-dimensional projection. Different lineages were indicated by different nuclear markers during analysis. Geometric observables as well as general distortions in projections can be correctly quantified using built-in correction methods in MATLAB.

### Numerical Simulations

### CPM

A CPM was used to infer the predicted distributions of conformations given measurements of cell adhesion from AFM, as well as to determine the roles of cortical stiffness on the self-organization of ETX embryos. Adhesion strengths were parameterized using cohesion forces between pairs of cell types, which were directly measured by AFM (Table 6). For each simulation, this distribution was sampled to build the adhesion (J) matrix. Specifically, for a given element in this matrix, the set of AFM cohesion forces measured between pairs of cell types (for example, ES-ES, ES-TS and so on) was sampled (with replacement), and performed around 500 times to establish an ensemble of J matrix samples. Each J matrix sample was used to perform a CPM simulation, generating an ensemble distribution of conformations over time. Simulations evolve via a stochastic minimization of an energy function (see equation (1) below) that accounts for both differential affinity and other physical properties of cells. Simulations were scored at each time point for being one of the 16 possible sorted configurations (FIG. 2I) by determining whether each cell type was enveloping and/or contiguous (see “Modeling” below for details). To test for the importance of softness in XEN cell externalization, the cortical stiffness parameter for XEN cells (λ^{XEN}) was varied and the above simulation procedure was repeated.

### Statistics and Reproducibility

Statistical tests were performed using GraphPad Prism (versions 8.0 and 7.0a) software (with the exception of the analysis of sequencing data). Data with a Gaussian distribution were analysed using a two-tailed unpaired Student's t-test (two groups) or one-way analysis of variance (ANOVA) (multiple groups) with Tukey's multiple comparison test. Significant differences in the variance were taken into account using Welch's correction. Data that did not have a Gaussian distribution were analysed using a Mann-Whitney U-test (two groups) or Kruskal-Wallis test (multiple groups) with Dunn's multiple comparison test. For all quantifications, a minimum of three independent experiments were performed. The in vitro cell experiments were not randomized as it was not necessary. For experiments with chemical inhibitors, samples were randomly allocated to control and experimental groups. Embryos were randomly allocated to control and experimental groups for the in vivo experiments.

Data collection and analysis were not performed blind to the conditions of the experiments. No statistical method was used to predetermine sample sizes.

Sample sizes were determined based on previous experimental experience. The sample sizes used to derive statistics are provided in the description. No data were excluded from the analyses. Sequencing data were analyzed using standard programs and packages. Significance levels are shown in each graph.

TABLE 3
ANTIBODIES

| Antibodies | SOURCE | IDENTIFIER |
|---|---|---|
| Goat polyclonal anti-Tfap2c | R&D Systems | Cat# AF5059; RRID: |
| (1:200) | | AB_2255891 |
| Goat polyclonal anti-Brachyury | R&D Systems | Cat# AF2085; RRID: |
| (1:200) | | AB_2200235 |
| Rabbit monoclonal anti-Gata4 | Cell Signalling Technology | Cat# 36966; RRID: |
| (1:500) | | AB_2799108 |
| Rabbit polyclonal anti-laminin | Sigma-Aldrich | Cat# L9393; RRID: |
| (1:500) | | AB_477163 |
| Mouse monoclonal anti-Oct4 | Santa Cruz Biotechnology | Cat# sc-5279; RRID: |
| (1:500) | | AB_628051 |
| Rat Monoclonal anti-E-cadherin | Thermo Fisher Scientific | Cat# 13-1900; RRID: |
| (1:200) | | AB_2533005 |
| Goat Monoclonal anti-P- | Santa Cruz Biotechnology | Cat# sc-1501; RRID: |
| cadherin (1:100) | | AB_630961 |
| Mouse Monoclonal anti-P- | Fisher Scientific | Cat# MS-1741; RRID: |
| cadherin (1:100) | | AB_149083 |
| Donkey anti-Mouse IgG (H + L), | Thermo Fisher Scientific | Cat# A-21202; RRID: |
| Alexa Fluor 488 (1:500) | | AB_141607 |
| Donkey anti-Goat IgG (H + L), | Thermo Fisher Scientific | Cat# A-11055; RRID: |
| Alexa Fluor 488 (1:500) | | AB_2534102 |
| Donkey anti-Rat IgG (H + L), | Thermo Fisher Scientific | Cat# A-21208; RRID: |
| Alexa Fluor 488 (1:500) | | AB_2535794 |
| Donkey anti-Rabbit IgG (H + L), | Thermo Fisher Scientific | Cat# A10042; RRID: |
| Alexa Fluor 568 (1:500) | | AB_2534017 |
| Donkey anti-Mouse IgG (H + L), | Thermo Fisher Scientific | Cat# A10037; RRID: |
| Alexa Fluor 568 (1:500) | | AB_2534013 |
| Donkey anti-Goat IgG (H + L), | Thermo Fisher Scientific | Cat# A-21447; RRID: |
| Alexa Fluor 647 (1:500) | | AB_2535864 |
| Alexa Fluor ™ Plus 405 | Thermo Fisher Scientific | Cat# A30104 |
| Phalloidin (1:200) | | |

TABLE 4
qRT-PCR PRIMERS

| | | SEQ | | SEQ |
|---|---|---|---|---|
| | Forward | ID | Reverse | ID |
| Gene | (5′ to 3′) | NO: | (5′ to 3′) | NO |
| Gapdh | CGTATTGGGC | 1 | ATGATGACCC | 2 |
| | GCCTGGTCAC | | TTTTGGCTCC | |
| Cdh1 | GGTTTTCTAC | 3 | GCTTCCCCAT | 4 |
| | AGCATCACCG | | TTGATGACAC | |
| Cdh3 | GCACCATGCA | 5 | AATATTGGTG | 6 |
| | GACAATGG | | GCATCACCCA | |
| | | | C | |
| Cdh6 | GATCCGATTA | 7 | TGTATGTCGC | 8 |
| | TCAGTACGTG | | CTGTGTTCTC | |
| | GG | | | |

TABLE 5A
FORWARD PCR PRIMERS

| Gene | | SEQ ID NO |
|---|---|---|
| Cdh1 | GGGGACAAGT | 9 |
| | TTGTACAAAA | |
| | AAGCAGGCTT | |
| | AATGGG | |
| | AGCCCGGTGC | |
| Cdh3 | GGGGACAAGT | 11 |
| | TTGTACAAAA | |
| | AAGCAGGCTT | |
| | AGCGAT | |
| | CGCCATGGAG | |
| | CT | |
| Cdh6 | ATATGGGCCC | 13 |
| | GCTTCTACAC | |
| | TCTCGGGGGC | |

TABLE 5B
REVERSE PCR PRIMERS

| Gene | | SEQ ID NO |
|---|---|---|
| Cdh1 | GGGGACCACT | 10 |
| | TTGTACAAGA | |
| | AAGCTGGGTT | |
| | CTAGTC | |
| | GTCCTCACCA | |
| | CCGC | |
| Cdh3 | GGGGACCACT | 12 |
| | TTGTACAAGA | |
| | AAGCTGGGTT | |
| | CACCGC | |
| | CACCATACAT | |
| | GTCC | |
| Cdh6 | ATATGCCGGC | 14 |
| | TTCTGCTCTG | |
| | TGCTTCTTGC | |

### Modeling

A Cellular Potts Model (CPM) was used to infer the predicted distributions of conformations given measurements of cell adhesion from AFM, and to determine the roles of cortical stiffness on self-organization of ETX-embryos. Embodiments of the modeling used herein are described below.

### Model Objects

Cells occupy contiguous sets of points in a square lattice of size (Nₓ×N_{y}). Each cell is prescribed a unique id, recorded in matrix I(Nₓ×N_{y}). Further, each cell is prescribed a cell type (e.g. ES, TS, XEN), entailing unique, pre-defined cellular properties. Cell type is immutable, establishing a mapping between the cell index i and its cell type cᵢ=2. Lattice points that are unoccupied by a cell define the medium, given an id i=0 and c₀=0.

### Energy Functional

The simulation evolves via a stochastic minimization of an energy function that accounts for both differential affinity and other physical properties of cells. The energy functional was defined as below:

$$E = {\sum }_{i = 1}^{{n}_{c}} {\underbrace{{{λ}_{A , i} ( {A}_{i} - {A}_{i , 0} )}^{2}}}_{Area penalty} + {\underbrace{{λ}_{P , i} ( {P}_{i}^{2} + κ {b}_{i} )}}_{Contractility} + {\underbrace{{λ}_{T} {\sum }_{x , y ∈ {ω}_{i}} {\sum }_{dx , dy ∈ Ω} {J}_{i , {I}_{x + dx , y + dy}}}}_{Adhesion / Tension} \qquad ( 1 )$$

λ_{A,i} describes the bulk modulus of area deformations of a cell i from its optimum A_{i,0}. λ_{P,i} defines its circumferential elastic modulus of the perimeter, scaling a contractility term (Pᵢ²) and the tension of interfaces between cells and the media (κbᵢ where bᵢ is the number of Moore neighbors of cell i that are medium). The final term accounts for adhesion/tension with neighboring cells: ωᵢ is the set of lattice points x, y that the cell occupies; Ω is the Moore neighborhood; meaning I_{x+dx,y+dy} is the cell id of a lattice point that neighbors a point within the cell; J_{i,I}_{_{x+dx,y+dy}} defines the strength of the interaction between cell i and the neighboring cell; and λ_{T} is a scale-factor across all adhesion terms. J is a symmetric matrix (n_{c}+1×n_{c}+1) of pairwise interaction strengths. Interactions must be between different cells, meaning Jᵢᵢ=0∀i. The matrix I defines the area and perimeters of each cell. The area Aᵢ of cell i is defined as the number of lattice point that cell i occupies, i.e.:

$${A}_{i} = {\sum }_{x = 1}^{{N}_{x}} {\sum }_{y = 1}^{{N}_{y}} {δ}_{i , {I}_{x , y}} \qquad ( 2 )$$

Likewise, the perimeter Pᵢ of cell i is the number of lattice points that are: (i) members of the Moore neighborhood of the lattice points of cell i (i.e. ωᵢ); but (ii) are not themselves members of the cell i.

### Bootstrapping Procedure

Adhesion strengths were parameterized using cohesion forces between pairs of celltypes that were directly measured by AFM. For each simulation, this distribution was sampled to build the J matrix. Specifically, for a given element Jᵢⱼ a user can sample (with replacement) the set of AFM cohesion forces measured between cell-types cᵢ and cⱼ (e.g. ES-ES, ES-TS, . . . ), while enforcing symmetry in the J matrix. Entries are set between cells and the medium (U₀ⱼ,Jᵢ₀) to 0. Bootstrap sampling is performed around 500 times to establish an ensemble of J matrix samples. Each J matrix sample is used to perform a CPM simulation, generating an ensemble distribution of conformations over time.

### Simulation Algorithm

The CPM evolves via a stochastic minimization. In each Markov Chain Step (MCS), a random lattice site is selected. One of the four sites in the Von Neumann neighborhood is then selected and the state of the chosen site is putatively reassigned to that of its neighbor. The energy functional is then evaluated before and after the swap, defining ΔE. The swap is then accepted only if:

$$Δ E = min ( 1 , exp ( \frac{- Δ E}{T} ) ) \qquad ( 3 )$$

As with the lattice model, T defines the effective temperature of the system, modulating the propensity to perform energetically unfavorable swaps. In traditional CPM simulations, cell Moore contiguity breaks down at high T given swapping rules are local. Consequently, potential state changes that compromise contiguity are universally rejected.

### Automated Scoring of Conformations

To determine the conformation of a simulated structure at a given time-point, an automated scoring procedure was established. Firstly, cells are removed that have detached from the main aggregate by calculating the adjacency matrix between cells (Moore neighborhood) and removing all clusters besides the one with the largest number of connected components. Secondly, each cell-type for envelopment is scored. A cell-type is defined to be enveloping if its center of mass lies within a different cell-type, rather than that of its own. Thirdly, cell-type contiguity is scored by calculating the subgraph of the connectivity matrix that contains only cells of a given type, then determining whether the number of connected components is 1 (i.e. contiguous). With three cell-types, there are 16 possible completely sorted conformations. These conformations can be divided into 4 categories.

In category (1) conformations, two cell types sequentially envelope a third. The order of envelopment is determined via adjacency among cell-types. For example, when E envelopes X which envelopes T: at least one X must contact T; at least one E must contact the medium; at least one E must contact X; and no E should contact T. Further, the inner most cell-type must be contiguous.

In category (2), one cell type envelopes another, with a third attached peripherally; whereas in category (3) one cell type envelopes the other two (as in ETX embryos). Both categories must contain two contiguous cell-types and a third enveloping celltype. If all cells of the enveloping cell-type contact the medium, the conformation is scored to category (3). If any of the cells that do not contact the medium are instead surrounded by a single cell-type, the conformation is scored as “unsorted”. Alternatively, if any of these cells contact exactly two other cell-types, then the conformation falls in category (2). Which variant within category (2) is determined by counting the number of contacts (e.g. X envelopes E rather than T if X and E share more contacts than X and T). Otherwise, the conformation is assigned category (3). Category (4) is assigned when all three cell-types are non-enveloping and are contiguous. If a given structure does not fall within any of these categories, it is classed as “unsorted”.

Additionally, cell externalization is defined: if all cells of that type either contact the medium directly, or are connected to cells that are connected to the medium. Strictly, the subgraph of the adjacency matrix containing the rows and columns of a given cell-type plus the medium is defined; if this subgraph has a single connected component, then the cell-type is externalized.

Lower Stiffness in XEN Cells Improves the Speed and Fidelity of their Externalization

The CPM was used to determine whether reduced stiffness in XEN cells can explain the robustness of their externalization in silico. The stiffness of XEN cells was systematically altered by varying the circumferential elastic modulus of XEN cells λ_{P}^{XEN} between 0.04 and 0.20 (9 values simulated). This parameter ascribes the extent of the circumferential energy penalty, meaning a cell with a higher values of λ_{P}^{XEN} resists deformations to its perimeter more i.e. is stiffer.

TABLE 6
PARAMETERS FOR THE CELLULAR POTTS MODEL

| Parameter | Value |
|---|---|
| Nₓ | 100 |
| N_{y} | 100 |
| N_{E} | 8 |
| N_{T} | 8 |
| N_{X} | 6 |
| A₀ | 30 |
| T | 15 |
| λ_{A} | 1 |
| λ_{P} | 0.2 |
| λ_{T} | −6 |
| κ | 3 |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of generating a synthetic embryo in vitro, the method comprising:
(a) providing a plurality of engineered embryonic stem cells (ESCs), wherein at least a portion of the plurality of engineered ESCs over-express E-cadherin (Cdh1);
(b) providing a plurality of engineered trophoblast stem cells (TSCs), wherein at least a portion of the plurality of engineered TSCs over-express P-cadherin (Cdh3);
(c) providing a plurality of extra-embryonic (XEN) cells; and
(d) contacting the plurality of engineered ESCs, the plurality of engineered TSCs and the plurality of extra-embryonic (XEN) cells with a first culture media to form a co-culture;
wherein the plurality of engineered ESCs and derivatives thereof, the plurality of engineered TSCs and derivatives thereof, and the plurality of XEN cells and derivatives thereof organize to form a synthetic embryo,
wherein the synthetic embryo comprises one TS-derived compartment and one ES-derived compartment, and is covered by an outside XEN-derived monolayer.

2. The method of claim 1, comprising (e) replacing the first culture media with a second culture media about three days after the contacting step (d).

3. The method of claim 1, wherein the plurality of engineered ESCs, the plurality of engineered TSCs, and the plurality of XEN cells organize into a multicellular aggregate structure within about 12-24 hours of the contacting step (d); and
wherein the multicellular aggregate structure develops into a multicellular aggregate structure comprising one TS-derived compartment and one ES-derived compartment, at least partially covered by an outside XEN-derived monolayer with an efficiency of about 30% after about 12 hours following the contacting step (d).

4. (canceled)

5. The method of claim 3, wherein the multicellular aggregate structure develops into the synthetic embryo with an efficiency of about 40% after at least 3 days following the contacting step (d).

6. The method of claim 5, wherein the synthetic embryo develops a single interior cavity with an efficiency of about 90%; optionally, wherein the single interior cavity develops between four and five days after the contacting step (d).

7. The method of claim 3, wherein the multicellular aggregate structure develops into the synthetic embryo and comprises a single interior cavity, with an efficiency of about 40%; optionally, wherein the single interior cavity develops between four and five days after the contacting of step (d).

8. The method of claim 3, wherein the multicellular aggregate structure develops into a synthetic embryo comprising a laminin-containing basement membrane with an efficiency of about 78%; optionally, wherein the laminin-containing basement membrane develops between four and five days after the contacting step (d).

9. (canceled)

10. The method of claim 1, wherein the synthetic embryo has a length of about 200 μm to about 500 μm, about 72 hours following the contacting step (d) and wherein the synthetic embryo has a size of about 6×10³ μm² to about 10×10³ μm², about 72 hrs following the contacting step (d).

11. (canceled)

12. (canceled)

13. The method of claim 1, wherein the TS-derived compartment comprises cells that express at least one TS cell-marker, optionally, wherein the at least one TS cell-marker comprises Tfap2C, EOMES, or both; wherein
the ES-derived compartment comprises cells that express at least one ES cell-marker, optionally, wherein the at least one ES cell-marker comprises Oct4; and wherein
the XEN-derived monolayer comprises cells that express at least one XEN cell-marker, optionally, wherein the at least one XEN cell-marker comprises Gata4, Gata6, or both.

14. (canceled)

15. (canceled)

16. The method of claim 1, wherein the synthetic embryo resembles an egg cylinder structure, after about three days following the contacting step (d); and/or
wherein the synthetic embryo resembles a post-implantation embryo structure, after about four to five days following the contacting step (d).

17. (canceled)

18. The method of claim 1, wherein providing the plurality of engineered ESCs comprises:
(i) providing an expression construct comprising a nucleic acid encoding E-cadherin, operably linked to at least one expression control element permitting gene expression in mammalian cells; and
(ii) introducing the expression construct into ESCs in a manner permitting expression of the introduced construct in at least one of the ESCs, thereby generating at least one engineered ESC.

19. (canceled)

20. The method of claim 1, wherein providing the plurality of engineered TSCs comprises:
(i) providing an expression construct comprising a nucleic acid encoding P-cadherin, operably linked to at least one expression control element permitting gene expression in mammalian cells; and
(ii) introducing the expression construct into TSCs in a manner permitting expression of the introduced construct in at least one of the TSCs, thereby generating at least one engineered TSC.

21.-24. (canceled)

25. The method of claim 1, wherein the at least a portion of the plurality of engineered ESCs over-express E-cadherin relative to wild-type ESCs; wherein the at least a portion of the plurality of engineered TSCs over-express P-cadherin relative to wild-type TSCs; and
wherein the plurality of XEN cells are wild-type XEN cells and none of the plurality of XEN cells are engineered to over-express E-cadherin, P-cadherin, or K-cadherin.

26-28. (canceled)

29. The method of claim 1, wherein the plurality of engineered ESCs comprises 6000-7000 ESCs, the plurality of engineered TSCs comprises 15000-19000 TSCs, and the plurality of XEN cells comprises 5000-6000 XEN cells.

30. (canceled)

31. (canceled)

32. The method of claim 1, wherein the ESCs, the TSCs, and/or the XEN cells are derived from a mouse or human natural embryo.

33. (canceled)

34. The method of claim 1, wherein the co-culturing is performed in an inverted pyramidal microwell and wherein the inverted-pyramidal microwell is about 400 μm or about 800 μm in size, optionally about 400 μm or about 800 μm diameter.

35. (canceled)

36. The method of claim 1, wherein the first culture media of step (d) comprises a ROCK inhibitor and wherein the method comprises removing the ROCK inhibitor following about 24 hr of co-culture in the first culture media.

37.-45. (canceled)

46. The method of claim 1, wherein the first culture media comprises DMEM, 12.5% FBS, 2 mM GlutaMax™, 0.1 mM 2-ME, 0.1 mM non-essential amino acids, 1 mM sodium pyruvate, 0.02 M HEPES, 1% Penicillin-streptomycin, and 7.5 nM ROCK inhibitor; or
wherein the first culture media comprises DMEM, 12.5% FBS, 2 mM GlutaMax™, 0.1 mM 2-ME, 0.1 mM non-essential amino acids, 1 mM sodium pyruvate, 0.02 M HEPES, and 1% Penicillin-streptomycin; optionally, wherein the first culture media does not comprise ROCK inhibitor.

47. (canceled)

48. (canceled)

49. The method of claim 2, wherein the second culture media comprises DMEM/F12, 20% FBS, 2 mM GlutaMax™, 1% Penicillin-streptomycin, 1×ITS-X, 8 nM β-estradiol, 200 ng/ml progesterone, and 25 mM N-acetyl-L-cysteine.

50. The method of claim 2, further comprising:
(f) replacing the second culture media with a third culture media about one day after step (e), wherein the third culture media comprises DMEM/F12, 30% KnockOut™ Serum Replacement, 2 mM GlutaMax™, 1% Penicillin-streptomycin, 1×ITS-X, 8 nM β-estradiol, 200 ng/ml progesterone, and 25 mM N-acetyl-L-cysteine.

51. (canceled)

52. (canceled)

53. A differentiated cell obtainable from a synthetic embryo, wherein the synthetic embryo is generated by a method comprising:
(a) providing a plurality of engineered embryonic stem cells (ESCs), wherein at least a portion of the plurality of engineered ESCs over-express E-cadherin (Cdh1);
(b) providing a plurality of engineered trophoblast stem cells (TSCs), wherein at least a portion of the plurality of engineered TSCs over-express P-cadherin (Cdh3);
(c) providing a plurality of extra-embryonic (XEN) cells; and
(d) contacting the plurality of engineered ESCs, the plurality of engineered TSCs and the plurality of extra-embryonic (XEN) cells with a first culture media to form a co-culture;
wherein the plurality of engineered ESCs and derivatives thereof, the plurality of engineered TSCs and derivatives thereof, and the plurality of XEN cells and derivatives thereof organize to form a synthetic embryo,
wherein the synthetic embryo comprises one TS-derived compartment and one ES-derived compartment, and is covered by an outside XEN-derived monolayer.

54. A method for determining the effect of a test agent on embryonic development, comprising:
i. providing a synthetic embryo generated by a method comprising:
(a) providing a plurality of engineered embryonic stem cells (ESCs), wherein at least a portion of the plurality of engineered ESCs over-express E-cadherin (Cdh1);
(b) providing a plurality of engineered trophoblast stem cells (TSCs), wherein at least a portion of the plurality of engineered TSCs over-express P-cadherin (Cdh3);
(c) providing a plurality of extra-embryonic (XEN) cells; and
(d) contacting the plurality of engineered ESCs, the plurality of engineered TSCs and the plurality of extra-embryonic (XEN) cells with a first culture media to form a co-culture;
wherein the plurality of engineered ESCs and derivatives thereof, the plurality of engineered TSCs and derivatives thereof, and the plurality of XEN cells and derivatives thereof organize to form a synthetic embryo,
wherein the synthetic embryo comprises one TS-derived compartment and one ES-derived compartment, and is covered by an outside XEN-derived monolayer;
ii. contacting the synthetic embryo with a test agent; and
iii. determining the effect of the test agent on the synthetic embryo, optionally the determining comprises comparing a phenotype or a genotype of the synthetic embryo in the presence of the test agent with the phenotype or genotype of the synthetic embryo in the absence of the test agent.

55.-62. (canceled)


SEQUENCE LISTING (14 sequences)

SEQ ID NO: 1 - DNA, 20 bp, synthetic construct
  source 1..20: mol_type = other DNA
cgtattgggcgcctggtcac

SEQ ID NO: 2 - DNA, 20 bp, synthetic construct
  source 1..20: mol_type = other DNA
atgatgacccttttggctcc

SEQ ID NO: 3 - DNA, 20 bp, synthetic construct
  source 1..20: mol_type = other DNA
ggttttctacagcatcaccg

SEQ ID NO: 4 - DNA, 20 bp, synthetic construct
  source 1..20: mol_type = other DNA
gcttccccatttgatgacac

SEQ ID NO: 5 - DNA, 18 bp, synthetic construct
  source 1..18: mol_type = other DNA
gcaccatgcagacaatgg

SEQ ID NO: 6 - DNA, 21 bp, synthetic construct
  source 1..21: mol_type = other DNA
aatattggtggcatcacccac

SEQ ID NO: 7 - DNA, 22 bp, synthetic construct
  source 1..22: mol_type = other DNA
gatccgattatcagtacgtggg

SEQ ID NO: 8 - DNA, 20 bp, synthetic construct
  source 1..20: mol_type = other DNA
tgtatgtcgcctgtgttctc

SEQ ID NO: 9 - DNA, 46 bp, synthetic construct
  source 1..46: mol_type = other DNA
ggggacaagtttgtacaaaaaagcaggcttaatgggagcccggtgc

SEQ ID NO: 10 - DNA, 50 bp, synthetic construct
  source 1..50: mol_type = other DNA
ggggaccactttgtacaagaaagctgggttctagtcgtcctcaccaccgc

SEQ ID NO: 11 - DNA, 48 bp, synthetic construct
  source 1..48: mol_type = other DNA
ggggacaagtttgtacaaaaaagcaggcttagcgatcgccatggagct

SEQ ID NO: 12 - DNA, 50 bp, synthetic construct
  source 1..50: mol_type = other DNA
ggggaccactttgtacaagaaagctgggttcaccgccaccatacatgtcc

SEQ ID NO: 13 - DNA, 30 bp, synthetic construct
  source 1..30: mol_type = other DNA
atatgggcccgcttctacactctcgggggc

SEQ ID NO: 14 - DNA, 30 bp, synthetic construct
  source 1..30: mol_type = other DNA
atatgccggcttctgctctgtgcttcttgc